(19) United States
(12) Patent Application Publication
(10) Pub. No.: US 2018/0344725 A1
(43) Pub. Date: Dec. 6, 2018

(54) QUINOLONE CHALCONE COMPOUNDS AND USES THEREOF

(71) Applicants: Hoyun Lee, Ontario (CA); Piyush Trivedi, Indore (IN); Chandrabose Karthikeyan, Sivakasi (IN); Indeewari Kalhari Lindamulage, Ontario (CA)

(72) Inventors: Hoyun Lee, Ontario (CA); Piyush Trivedi, Indore (IN); Chandrabose Karthikeyan, Sivakasi (IN); Indeewari Kalhari Lindamulage, Ontario (CA)

(21) Appl. No.: 15/777,468

(22) PCT Filed: Nov. 18, 2016
(86) PCT No.: PCT/CA2016/051349

§ 371 Date: May 18, 2018

Related U.S. Application Data
(60) Provisional application No. 62/258,033, filed on Nov. 20, 2015.

(51) Int. Cl. A61K 31/4704; A61K 31/337; A61P 35/00; C07D 215/227

(52) U.S. Cl. CPC A61K 31/4704; C07D 215/227; A61P 35/00; A61K 31/337

(57) ABSTRACT

The present disclosure relates to novel compounds, compositions comprising these compounds, and their use, for example for the treatment of cancer. In particular, the present disclosure includes compounds of Formula (I), and compositions and uses thereof.

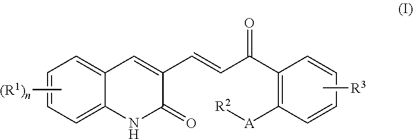

### CROSS-REFERENCE TO RELATED APPLICATION

This application claims the benefit of U.S. Provisional Patent Application Ser. No. 62/258,033, filed Nov. 20, 2015, the content of which is hereby incorporated by reference in its entirety.

### FIELD

The present disclosure relates to novel quinolone chalcone compounds, compositions comprising such novel quinolone chalcone compounds, and their use, for example, for the treatment of cancer.

### BACKGROUND

Continuously dividing cancer cells are dependent upon the rapid and dynamic process of the polymerisation and depolymerisation of tubulin.

Microtubule targeting agents such as paclitaxel and vinblastine have been widely used at clinics (Dumontet and Jordan, 2010; Kuppens, 2006; Singh et al., 2008). Microtubule targeting agents are known to bind tubulin via at least four different binding sites/areas. Paclitaxel binds to the inner surface of the β-subunit of polymerized tubulin, resulting in the stabilization of microtubule structure and thus preventing depolymerisation (Lu et al., 2012). The Laulimalides cause microtubule stabilization similar to taxanes, although they bind to a different site (Pryor et al., 2002). Vinca alkaloids bind to a few tubulin subunits at the end of the polymer, preventing them from undergoing polymerisation. However, vinblastine is also capable of binding at the interface of two αβ-tubulin heterodimers, thus preventing self-association (Gigant et al., 2005). The fourth group of microtubule targeting agents bind to tubulin through the colchicine binding site. This class of compounds binds to the β-tubulin subunit, resulting in the inhibition of microtubule assembly (Ravelli et al., 2004). Although it inhibits microtubule assembly, the therapeutic value of colchicine is limited due to its low therapeutic index (i.e., high toxicity).

Unlike taxanes and vinca alkaloids, agents targeting the colchicine binding site have minimal multidrug resistance issues. Therefore, many efforts have been undertaken to develop drugs that effectively bind to the colchicine binding site with minimal side effects (Borisy and Taylor, 1967a; Borisy and Taylor, 1967b; Lu et al., 2012; Weisenberg et al., 1968; Zhou and Giannakakou, 2005). However, to date, an effective drug targeting the colchicine-binding site with low side effects has not yet been approved by US FDA.

### SUMMARY

It was found that the quinolone chalcones of the studies disclosed herein, as exemplified by the compounds CTR-17 [(E)-3-(3-(2-methoxyphenyl)-3-oxoprop-1-enyl) quinolin-2(1H)-one] and CTR-20 [(E)-6-methoxy-3-(3-(2-methoxyphenyl)-3-oxoprop-1-enyl) quinolin-2(1H)-one], bind to tubulin at the colchicine binding site, leading to cell killing in a cancer-specific manner. Both of these CTR compounds were observed to effectively kill multidrug-resistant (including paclitaxel-, vinblastine- and colchicine-resistant) cancer cells. Furthermore, the data obtained in the present studies also showed that the combination of paclitaxel and CTR-17 or CTR-20 has strong synergistic effects on multidrug-resistant cells. The data from animal studies showed that the CTR compounds tested, alone or in combination with paclitaxel, possess strong antitumor activity without notable ill-effects to animals observed. Further, CTR-21 ((E)-8-methoxy-3-(3-(2-methoxyphenyl)-3-oxoprop-1-enyl) quinolin-2(1H)-one) and CTR-32 ((E)-3-(3-(2-ethoxyphenyl)-3-oxoprop-1-enyl)quinolin-2(1H)-one) are also highly potent in tumor cell killing. Like CTR-17 and CTR-20, CTR-21 and CTR-32 also disrupt the microtubule dynamics. Data from three isogenic cell lines showed that CTR-20, CTR-21 and CTR-32 preferentially kill the fully malignant MCF10CA1a breast cancer cells over premalignant MCF10AT1 and the non-malignant MCF10A breast cells. Furthermore, all of these compounds effectively kill multidrug-resistant cancer cells.

Accordingly, the present disclosure includes a compound of Formula I:

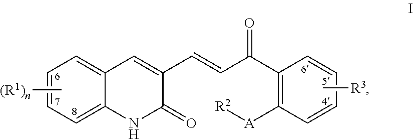

wherein

A is O or S;

n is 0, 1, 2 or 3;

when n is 1, R¹ is halo, C₁₋₆alkyl, C₂₋₆alkenyl or —X—C₁₋₆alkyl;

when n is 2 or 3, each R¹ is independently halo, C₁₋₆alkyl, C₂₋₆alkenyl or —X—C₁₋₆alkyl; or two R¹ together form a methylenedioxy group that is attached to two adjacent ring carbon atoms;

R² is C₁₋₆alkyl or C₁₋₆haloalkyl;

R³ is absent or is halo, —X—C₁₋₆alkyl or —X—C₁₋₆haloalkyl; and

each X is independently O or S,

or a pharmaceutically acceptable salt, solvate and/or prodrug thereof.

In an embodiment, A is O.

In an embodiment, R³ is absent.

In an embodiment, R² is methyl.

In an embodiment, n is 1 and R¹ is 6-OCH₃, 7-OCH₃, 8-OCH₃, 6-OC₂H₅, 6-SCH₃, 7-SCH₃, 6-CH₃, 6-C₂H₅, 6-F, 6-Cl, 6-Br, 7-F, 7-Cl or 7-Br. In another embodiment, n is 1 and R¹ is 6-CH₃, 6-OCH₃ or 7-OCH₃.

In an embodiment, n is 2 and R¹ is 6,7-diCH₃, 6,7-diOCH₃ or 6,7-O—CH₂—O—. In another embodiment, n is 3 and R¹ is 5,6,7-triOCH₃.

In an embodiment, the compound is selected from:

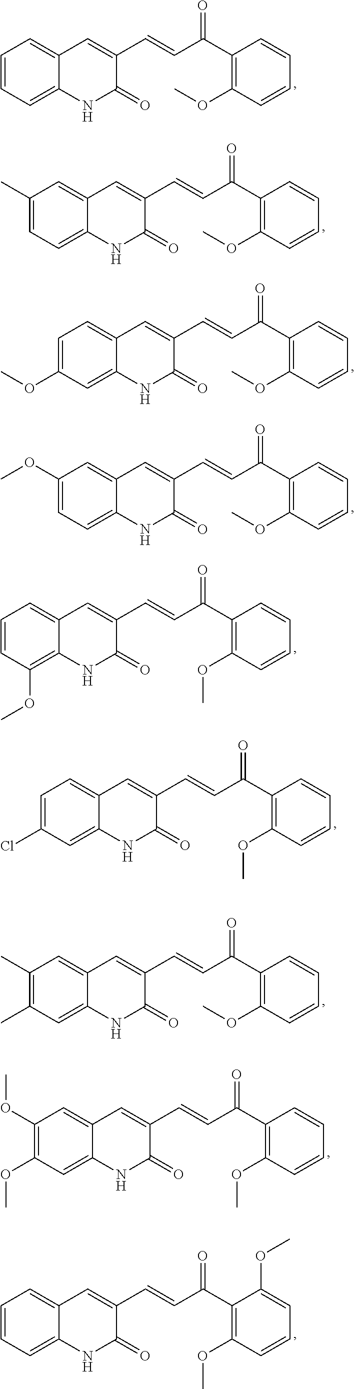

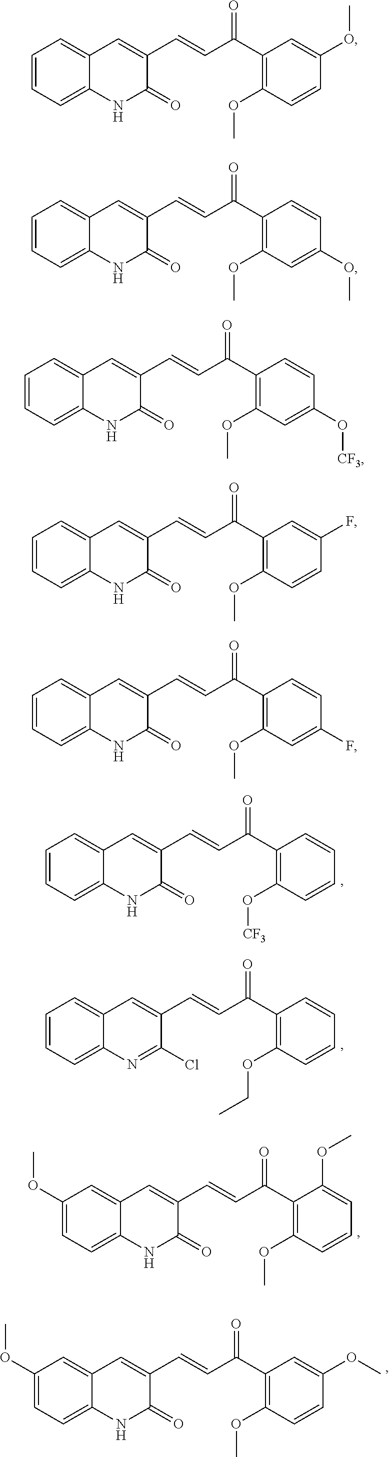

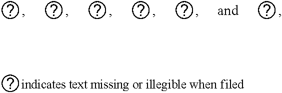

[symbol]

or a pharmaceutically acceptable salt, solvate and/or prodrug thereof.

In another embodiment, the compound is:

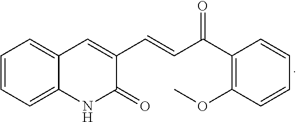 [SMILES: COc1ccccc1C(=O)/C=C/c1cc2ccccc2[nH]c1=O]

In a further embodiment, the compound is:

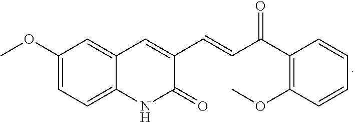 [SMILES: COc1ccc2[nH]c(=O)c(/C=C/C(=O)c3ccccc3OC)cc2c1]

The present disclosure also includes a pharmaceutical composition comprising one or more compounds of the present disclosure and a pharmaceutically acceptable carrier.

The present disclosure also includes a method of treating cancer comprising administering one or more compounds of the present disclosure to a subject in need thereof.

In an embodiment, the cancer is breast cancer, leukemia, cervical cancer, brain cancer, lung cancer, bladder cancer, kidney cancer, multiple myeloma or other blood cancers, colorectal cancer, CNS cancer, melanoma, ovarian cancer and prostate cancer. In another embodiment, the cancer comprises colchicine-resistant, paclitaxel-resistant, bortezomib-resistant, vinblastine-resistant and/or multidrug-resistant tumor cells.

In an embodiment, the one or more compounds of the present disclosure are administered in combination with one or more other anticancer agents. In another embodiment, the other anticancer agents are selected from the group consisting of mitotic inhibitors, optionally paclitaxel; bcl2 family inhibitors, optionally ABT-737 and other inhibitors of the anti-apoptotic pathway; proteasome inhibitors, optionally bortezomib or calfilzomib; signal transduction inhibitors, optionally gefitinib, erlotinib, dasatinib, imatinib or sunitinib; inhibitors of DNA repair, optionally iniparib, temozolomide or doxorubicin; and alkylating agents, optionally cyclophosphamide. In a further embodiment, the other anticancer agent is paclitaxel.

In embodiments wherein the one or more compounds of the present disclosure are administered in combination with one or more other anticancer agents, the dosage of the one or more compounds of the present disclosure is optionally less than the dosage of the one or more compounds of the present disclosure when administered alone. In another embodiment, the dosage of the one or more compounds of the present disclosure is one half the dosage of the one or more compounds of the present disclosure when administered alone.

In embodiments wherein the one or more compounds of the present disclosure are administered in combination with one or more other anticancer agents, the dosage of the other anticancer agent is optionally less than the dosage of the other anticancer agent when administered alone. In another embodiment, the dosage of the other anticancer agent is one half the dosage of the other anticancer agent when administered alone.

### BRIEF DESCRIPTION OF THE DRAWINGS

The present disclosure will now be described in greater detail with reference to the drawings, in which:

FIG. 1 is a plot showing percentage cell death of asynchronously growing HeLa S3 cells treated with the compound CTR-20 ((E)-6-methoxy-3-(3-(2-methoxyphenyl)-3-oxoprop-1-enyl) quinolin-2(1H)-one) at concentrations of 0, 0.5, 1.0, 5.0 and 10.0 μM for 24 or 72 hours (h).

FIG. 2 shows (A) flow cytometry profiles of HeLa cells at 72 hours (h) post-treatment with different concentrations (0, 0.75, 1.0, 3.0, 5.0, 7.5 and 10.0 μM) of the compound CTR-17 ((E)-3-(3-(2-methoxyphenyl)-3-oxoprop-1-enyl) quinolin-2(1H)-one); and (B) cell cycle profiles at different time points (6, 12, 24, 48 and 72 hours) after asynchronous HeLa cells were treated with 3.0 μM CTR-17 (bottom row) in comparison to sham-treated HeLa cells (top row).

FIG. 3 shows flow cytometry profiles which were taken at different times (0, 6, 12, 24, 48 and 72 hours) after two breast cancer cell lines (MDA-MB-468 and MDA-MB-231; top and middle row, respectively) and one non-cancer breast cell line (MCF-10A) were treated with 3.0 μM CTR-17 (bottom row).

FIG. 4 shows flow cytometry profiles taken after asynchronous breast cancer cells (MDA-MB-231 and MCF-7; top and middle row, respectively) and their matching non-cancer breast cells (184B5; bottom row) were treated with CTR-20 at 0.5 or 1 μM for 72 hours (h) in comparison to untreated cells.

FIG. 5 shows flow cytometry profiles which were taken after (A) MDA-MB-231 cells were treated with 1 μM CTR-20 for 4, 8, 24, 28 and 72 hours in comparison to a sham control or (B) HeLa S3 cells were treated with 0.5, 1.0, or 2.5 μM CTR-20 for 24, 48 and 72 hours in comparison to a sham control.

FIG. 6 shows flow cytometry profiles that CTR-21 at >60 nM concentrations caused mitotic arrest and, eventually, cell death. HeLa cells were treated with different concentrations of CTR-21 for 6, 12, 24, 48 and 72 hours (h) prior to analysis of their cell cycle profiles by flow cytometry.

FIG. 7 shows flow cytometry profiles that CTR-32 at >50 nM concentrations caused mitotic arrest and, eventually, cell death. HeLa cells were treated with different concentrations of CTR-32 for 6, 12, 24, 48 and 72 hours (h) prior to analysis of their cell cycle profiles by flow cytometry.

FIG. 8 shows flow cytometry profiles that CTR-21 (30 nM) and CTR-32 (50 nM) do not cause notable effects on the cell cycle progression of the MCF10A non-cancer cells, except a transient mitotic arrest at the 12-hour time point (2^{nd} column from the left).

FIG. 9 illustrates that CTR-17, CTR-20, CTR-21 and CTR-32 preferentially kill the fully malignant MCF10Ala breast cancer cells over the pre-malignant MCF10AT1 and the non-malignant MCF-10A breast cells. Cells were treated with (A) CTR-17 at doses of 0.10, 0.39, 1.56 or 6.25 μM; (B) CTR-20 at doses of 0.10, 0.39, 1.56 or 6.25 μM; (C) CTR-21 at doses of 7.81, 15.63, 31.25, 62.5, 125 or 250 nM; or (D) CTR-32 at doses of 7.81, 15.63, 31.25, 62.5, 125 or 250 nM for 72 hours. Cell viability was determined using an SRB assay.

FIG. 10 shows exemplary images of (A) HeLa cells that were sham-treated or treated with 3 μM CTR-17 and stained with an antibody specific for γ-tubulin (far left), stained with an antibody specific for α-tubulin (second from left), counterstained DNA with DAPI (second from right) and merged images (far right). Scale bar denotes 10 μm; (B) of a higher magnification of the DAPI and merged images of HeLa cells that were sham-treated or treated with 3 μM CTR-17; and (C) of HEK293T, MDA-MB-468 and MDA-MB-231 cells that were treated with 3 μM CTR-17 and stained with an antibody specific for γ-tubulin (far left), stained with an antibody specific for α-tubulin (second from left), counterstained DNA with DAPI (second from right) and merged images (far right). Scale bar denotes 2 μm for images in top and middle row and 5 μm for images in bottom row. White arrows in (B) and (C) denote the failure of proper alignment at the centre plate or uneven segregation of chromosomes.

FIG. 11 is a plot showing the percentage of mitotic cells for non-cancer cells MCF-10A and 184B5 and cancer cells MDA-MB-231, HeLa, MDA-MB-468 and HEK293T that were either sham-treated or treated with 3.0 μM CTR-17 for 12 hours (h) or 24 hours, followed by analyses of cell cycle progression, centrosome abnormalities, and chromosome alignment/segregation.

FIG. 12 shows exemplary images of (A) approximately metaphase and (B) approximately anaphase and telophase/cytokinesis for asynchronously growing MCF-7, MDA-MB-231 and HeLa S3 cells treated with sham (MCF-7 only) or 1 μM CTR-20 for 24 hours (h). Cells were then collected, fixed with methanol, and incubated with an antibody specific for α-tubulin (far left column of FIGS. 12A and 12B) and then counterstained DNA with DRAQ5 (second from the left column of FIGS. 12A and 12B) and merged images (far right column of FIG. 12A; second from right and far right column of FIG. 12B). The internal box in the telophase/cytokinesis MCF-7 sample in FIG. 12B shows uneven cell division. Scale bars on all images except for the internal box denote 5 μm.

FIG. 13 is a plot showing the percentage of mitotic cells for non-cancer cells MCF-10A and 184B5 compared to cancer cells MDA-MB-231, HeLa and MCF-7 sham treated or treated with 1 μM CTR-20 for 24 hours.

FIG. 14 shows plots of the percentage of cells in various cell stages (from left to right on each of FIGS. 14A and 14B: prometaphase, metaphase, anaphase/telophase and cytokinesis) for HeLa S3 cells growing on cover slips synchronized by double thymidine treatment then released into fresh medium either in the absence (A; sham treated) or (B) presence of 1.0 μM CTR-20 for a duration of 7.5, 8.5, 9.5, 10, 10.5 or 11.5 hours.

FIG. 15 shows that chromosomes in HeLa cells are misaligned in the presence of CTR-21 (middle row) and CTR-32 (bottom row). HeLa cells were treated with 30 nM CTR-21 or 50 nM CTR-32 for 12 hours prior to fixing in methanol and immunostaining with an antibody specific for γ-tubulin (green; first column) or α-tubulin (red; second column), which were then counterstained with Draq5 (blue; third, fifth and seventh columns). Merged images are shown on the forth, sixth and eighth columns from the left. Failure to align properly at the center plate and perturbed separation of chromosomes are shown by white arrows.

FIG. 16 shows that CTR-21 and CTR-32 activate Bcl-X_{L} in the cells arrested at mitosis. HeLa cells were treated with CTR-21 (15 or 30 nM) or CTR-32 (30 or 50 nM) for 6, 12 or 24 hours (h). Whole cell lysates were collected at the scheduled time points and used to perform SDS PAGE protein separation, followed by Western blotting with antibodies specific for proteins listed on the right of the panel. p-S62 Bcl-X_{L} denotes Bcl-X_{L} phosphorylated on the serine 62 residue (i.e., Bcl-X_{L} is activated). GAPDH was used as the loading control. It should be noted that the levels of cyclin B and the high molecular weight (i.e., phosphorylated) of Cdc25C are much higher in the presence of CTR-21 or CTR-31 than in the absence either of the compounds.

FIG. 17 shows (A) exemplary HeLa cell cycle histograms after treatment with CTR-17 (3 μM; second from right) or CTR-20 (1 μM; far right) for 12 hours (which is defined as time 0 post-release) in comparison to untreated (far left) and sham treatment (second from left); and (B) exemplary cell cycle histograms of cells treated with CTR-17 (top) and CTR-20 (bottom) as described for FIG. 17A then washed twice with 1×PBS and re-suspended in 10 ml of pre-warmed, drug-free medium for durations of 3, 6, 9 or 12 hours (from left to right).

FIG. 18 shows that the effects of CTR-21 and CTR-32 are reversible. (A) HeLa cells arrested at G2/M phase by treating them with CTR-21 (30 nM) or CTR-32 (50 nM) for 12 hours (which is designated as time 0 h) were washed twice with 1×PBS and then released into cell cycle by culturing in drug-free medium for 1, 2, 4, 6 and 8 hours (h). The cell cycle progression was then analysed by flow cytometry (lower panels). (B) Exemplary images of HeLa cells at 1, 2, 4, 6 and 8 hours post release from the CTR treatment as shown in FIG. 18A.

FIG. 19 shows the results of Western blotting carried out with an anti-PARP antibody at time points (hours, h) of 12, 24 and 48 hours using whole cell extracts prepared from asynchronous HeLa cells (left) and 184B5 cells (right) treated with sham or 3 μM of CTR-17 (top image). GAPDH was used as a loading control (bottom image).

FIG. 20 shows (A) exemplary images of synchronous HeLa cells treated with CTR-17 (3.0 μM) for 24 hours, then fed with EdU (10.0 μM) for 1 hour immediately prior to harvesting them for analysis (bottom row) in comparison to a sham control (top row); and (B) exemplary images of cell immunostaining with an antibody specific for γ-H2AX (second column from left) to detect damaged DNA (i.e., damage repairing) of CTR-17 treated HeLa cells (bottom row) in comparison to a sham control (top row). Etoposide (50.0 μM) was used as a positive control (middle row). Scale bar denotes 20 μM.

FIG. 21 shows images of Western blots carried out with whole cell extracts prepared from asynchronously growing HeLa cells. Equal amounts of proteins were resolved by SDS-PAGE, and blotting was carried out with antibodies specific for those proteins listed at left of the gels (from top to bottom: p-Cdk1, Y15; pCdk1, T161; Cdk1; Cyclin B; Wee1; Cdc25C; p-Cdc25C, S216; pCdc25C, T48). Time points in hours (h) are post-treatment with 3.0 μM CTR-17 (right 4 columns) or sham control (left 5 columns). GAPDH (bottom image) was used as a loading control. “p-” denotes phosphorylation.

FIG. 22 shows flow cytometry profiles of HeLa cells synchronised at the G1/S border by double thymidine (DT) block then released into the cell cycle in the absence (sham; top row) or presence of CTR-17 (3.0 μM; bottom row) for a duration of 3, 6, 9, 12, 16 or 48 hours (h) in comparison to controls.

FIG. 23 shows the results of analysis of HeLa cells synchronized at the G1/S border by double thymidine (DT) block then released into complete medium at time 0 in the absence (sham; FIG. 23A) or presence (FIG. 23B) of 3.0 μM CTR-17 for a duration of 1, 3, 6, 9, 12, 14, 16, 18 or 20 hours (from left to right). Equal amounts of proteins were resolved by SDS-PAGE, followed by Western blotting with antibodies specific for the proteins, from top to bottom: p-Cdk1, Y15; pCdk1, T161; Cdk1; p-Cdc25C, T48; Cdc25C; securin; cyclin B; cyclin E; cyclin A; p-histone H3; histone H3; GAPDH (loading control); BubR1; and GAPDH (loading control). “p-” denotes phosphoprotein.

FIG. 24 shows the Western blot results of HeLa cells synchronised at the G1/S boundary by double thymidine (DT) block then sham treated (left three columns), treated with 20 ng/ml nocodazole (middle three columns), or treated with 3.0 μM CTR-17 (right three columns) for durations of 6, 9 and 12 hours (h). Total protein extracts were subjected to immunoprecipitation with an anti-BubR1 antibody, followed by protein separation by SDS-PAGE and Western blotting with an anti-Cdc20 antibody to examine the interaction between BubR1 and Cdc20.

FIG. 25 shows exemplary images of asynchronously growing HeLa cells sham-treated (top two rows) or treated with CTR-17 (3.0 μM; bottom two rows) for 12 hours, fixed, and then immunostained with antibodies specific for BubR1 (far left column) or Cenp-B (second from left column) (centromere staining). The column second from the right shows merged images and the far right column shows bright field images. The scale bar denotes 5 μm for the top row and second row from the bottom and denotes 2 μm for the other images.

FIG. 26 is a plot of absorbance at 340 nm as a function of time (min) after purified porcine tubulin and 1.0 mM GTP were added to a reaction mixture containing 10.0 μM paclitaxel, 3.0 μM CTR-17, 1.0 μM CTR-20, or 5.0 μM nocodazole then polymerization of tubulin was monitored every minute for one hour at 340 nm and 37° C. by spectrophotometry.

FIG. 27 shows that CTR-21 and CTR-32 effectively inhibit microtubule polymerization. Paclitaxel, CTR-20, CTR-21, CTR-32 or colchicine was added to a reaction mixture containing highly purified porcine tubulin and 1.0 mM GTP. The reaction was carried out for 1 hour at 37° C., while monitoring fluorescence emission at 1-minute intervals. The fluorescence excitation was at 350 nm and the emission was recorded at 430 nm.

FIG. 28 shows (A) the results of HeLa cells that were sham-treated (Sham), treated with 50.0 nM paclitaxel (Tax), 50.0 ng/ml nocodazole (Noc), 3.0 μM CTR-17, or 1.0 μM CTR-20 for 12 hours then the cell lysates were separated into polymerization (Pol) and soluble (Sol) fractions, and equal amounts of proteins resolved by SDS-PAGE, followed by immunoblotting with an antibody specific for α-tubulin (upper panel). Bands were quantified with densitometry and expressed in a graph form (lower panel). (B) HeLa cells treated with different concentrations of CTR-17 or CTR-20 and subjected to fractionation and immunoblotting as described for FIG. 28A.

FIG. 29 shows plots demonstrating that CTR-17 (A) and CTR-20 (B) quenched the intrinsic tryptophan fluorescence of tubulin in a dose-dependent manner. Purified tubulin dissolved in 25 mM PIPES buffer was incubated in the presence or absence of different concentrations of CTR compounds for 30 minutes at 37° C. Fluorescence was monitored by excitation of the reaction mixture at 295 nm, and the emission spectra were recorded from 315 to 370 nm; and plots of the change in fluorescence intensity as a function of drug concentrations of CTR-17 (C) and CTR-20 (D) to determine the dissociation constant. ΔF is the change in fluorescence intensity of tubulin when bound by the CTR compounds. Data are an average of five independent experiments.

FIG. 30 shows results suggesting that (A) CTR-17 and CTR-20, similar to colchicine, did not bind to the vinblastine binding site on the tubulin. 25 μM each of colchicine, CTR-17, CTR-20, or vinblastine was incubated with tubulin for 1 hour to promote the formation of complexes between tubulin and each of these compounds. The resultant complexes were incubated for 30 minutes with 5 μM of the fluorescent BODIPY FL-vinblastine to determine if the binding of each compound to tubulin is in competition with vinblastine. (B) CTR-17 binds to tubulin at or near the colchicine-binding site. The tubulin-fluorescent colchicine complex was incubated with increasing concentrations of either vinblastine or CTR-17. CTR-17 but not vinblastine competed with (fluorescent) colchicine. CTR-17 (C) and CTR-20 (D) depressed the fluorescence of the colchicine-tubulin complex in a dose-dependent manner. Tubulin was incubated with different concentrations of CTR-17 or CTR-20 for 1 hour, in three separate sets with concentrations of colchicine of 3.0, 5.0 and 8.0 μM (for CTR-17) or 1.0, 3.0 and 5.0 μM (for CTR-20). Inhibitory constants of CTR-17 (E) and CTR-20 (F). The fluorescence intensity of the final tubulin complex (FIGS. 30C and 30D) was used to determine the inhibitory concentration (Ki) utilizing a modified Dixon plot. F is the fluorescence of the complexes of CTR-17 (or CTR-20)-colchicine-tubulin or vinblastine-colchicine-tubulin complex, and F0 is the fluorescence of the colchicine-tubulin complex. Data are an average of at least four independent experiments.

FIG. 31 shows images of (A) the results of molecular docking predicting the tubulin-binding sites of colchicine, CTR-17, CTR-20, podophyllotoxin and vinblastine using the 3D X-ray structure of tubulin (PDB code: 1SA0); and (B) the chemical structures of colchicine, CTR-17, CTR-20 and podophyllotoxin.

FIG. 32 shows images of the predicted interaction between the tubulin heterodimer (PDB code: 1SA0) and colchicine (A), CTR-20 (B), or CTR-17 (C) in a 3D pattern. 2D ligand interaction diagrams show potential chemical interactions between amino acids and compounds within a distance of 4 Å to colchicine (A′), CTR-20 (B′), or CTR-17 (C′).

FIG. 33 shows images of (A) Western blotting of whole cell extracts prepared from the parental KB-3-1 and MDR1-overexpressing KB-C-2 isogenic cell lines; and (B) Western blotting of whole cell extracts prepared from the parental H69 and MRP1-overexpressing H69-AR isogenic cell lines.

FIG. 34 shows (A) part of the CTR-17 data presented in Table 8 in a graph form; and (B) part of the CTR-20 data presented in Table 8 in a graph form. CI denotes combination index. CI<1.0, CI=1.0 and CI>1.0 are synergistic, additive and antagonistic, respectively (Chou, 2006). Data presented are mean±S.E.M value of triplicates of at least four independent experiments.

FIG. 35 shows that CTR compounds kill multi-drug resistant and sensitive cells with similar efficacy. (A) The multidrug-resistant MDA-MB231TaxR cells express high levels of P-glycoprotein (P-gp; MDR1). Whole cell extracts of MDA-MB231 cells selected at different concentrations of paclitaxel (2.0, 10.0, 15.0, 30.0 and 100.0 nM) along with the parental MDA-MB231 (WT) were subjected to SDS-PAGE and Western blotting with an anti-MDR1 antibody. GAPDH was used as a loading control. (B) MDR1-overexpressing MDA-MB231TaxR (selected at 100 nM paclitaxel) and its parental MDA-MB231 cells were killed by CTR compounds with similar efficacy, while the MDA-MB231TaxR is over 114-fold more resistant to paclitaxel and at least 15-fold more resistant to vinblastine than the MDA-MB231.

FIG. 36 shows that CTR-20, CTR-21 and CTR-32 kill bortezomib-resistant RPMI-8226 cells (RPMI-8226BTZR) with similar potency to the parental RPMI-8226 multiple myeloma cells.

FIG. 37 shows that CTR-20, CTR-21 and CTR-32 are synergistic in killing the multidrug-resistant MDA-MB231TaxR (selected in 100 nM) cells when used in combination with paclitaxel. (A) Synergistic effects of CTR-20 in combination with paclitaxel against MDA-MB231TaxR. Lanes denote: 300 nM paclitaxel (Tax) (lane 1), 312.5 nM CTR-20 (lanes 2, 5, 8 & 11), 312.5 nM CTR-20 plus 300 nM paclitaxel (lane 3), 150 nM paclitaxel (lane 4), 312.5 nM CTR-20 plus 150 nM paclitaxel (lane 6), 75 nM paclitaxel (lane 7), 312.5 nM CTR-20 plus 75 nM paclitaxel (lane 9), 37.5 nM paclitaxel (lane 10), and 312.5 nM CTR-20 plus 37.5 nM paclitaxel (lane 12). (B) Synergistic effects of CTR-21 in combination with paclitaxel (Tax) against MDA-MB231TaxR (selected in 100 nM). Lanes denote: 300 nM paclitaxel (Tax) (lane 1), 23 nM CTR-21 (lanes 2, 5, 8, 11 & 14), 23 nM CTR-21 plus 300 nM paclitaxel (lane 3), 150 nM paclitaxel (lane 4), 23 nM CTR-21 plus 150 nM paclitaxel (lane 6), 75 nM paclitaxel (lane 7), 23 nM CTR-21 plus 75 nM paclitaxel (lane 9), 37.5 nM paclitaxel (lane 10), 23 nM CTR-21 plus 37.5 nM paclitaxel (lane 12) and 18.75 nM paclitaxel (lane 13), 23 nM CTR-21 plus 18.75 nM paclitaxel (lane 15). (C) Synergistic effects of CTR-32 in combination with paclitaxel against MDA-MB231TaxR (selected in 100 nM). Lanes denote: 300 nM paclitaxel (lane 1), 23 nM CTR-32 (lanes 2, 5, 8, 11 & 14), 23 nM CTR-32 plus 300 nM paclitaxel (lane 3), 150 nM paclitaxel (lane 4), 23 nM CTR-32 plus 150 nM paclitaxel (lane 6), 75 nM paclitaxel (lane 7), 23 nM CTR-32 plus 75 nM paclitaxel (lane 9), 37.5 nM paclitaxel (lane 10), 23 nM CTR-32 plus 37.5 nM paclitaxel (lane 12) and 18.75 nM paclitaxel (lane 13), 23 nM CTR-32 plus 18.75 nM paclitaxel (lane 15). “CI” denotes combinational index: CI<1.0, CI=1.0 and CI>1.0 are synergistic, additive and antagonistic, respectively. Data presented are mean±S.E.M value of triplicates of at least three independent experiments.

FIG. 38 shows that CTR-20 is synergistic when used in combination with ABT-737 against MDA-MB231 cells. (A) Lanes denote: 6.25 μM ABT-737 (lanes 1, 4 & 7), 0.4 μM CTR-20 (lane 2), 0.4 μM CTR-20 plus 6.25 μM ABT-737 (lane 3), 0.2 μM CTR-20 (lane 5), 0.2 μM CTR-20 plus 6.25 μM ABT-737 (lane 6), 0.1 μM CTR-20 (lane 8) and 0.1 μM CTR-20 plus 6.25 μM ABT-737 (lane 9). (B) Lanes denote: 3.125 μM ABT-737 (lanes 1, 4 & 7), 0.4 μM CTR-20 (lane 2), 0.4 μM CTR-20 plus 3.125 μM ABT-737 (lane 3), 0.2 μM CTR-20 (lane 5), 0.2 μM CTR-20 plus 3.125 μM ABT-737 (lane 6), 0.1 μM CTR-20 (lane 8) and 0.1 μM CTR-20 plus 3.125 μM ABT-737 (lane 9). CI denotes combinational index. Data presented are mean±S.E.M value of triplicates of at least three independent experiments.

FIG. 39 shows flow cytometry profiles of CTR-20, ABT-737 and the combination of the two against MDA-MB231. MDA-MB231 cells were sham treated (Sham) or treated with 6.25 μM ABT-737, 3.13 μM ABT-737, 0.4 μM CTR-20 (CTR), 0.4 μM CTR-20 plus 6.25 μM ABT-737 or 0.4 μM CTR-20 plus 3.13 μM ABT-737 for 6, 12, 24, 48 or 72 hours (h). Note that the combination of 0.4 μM CTR-20 plus 6.25 μM ABT-737 completely killed MDA-MB231 by 72 hours of treatment.

FIG. 40 shows Western blot data indicating that CTR-20 in combination with ABT-737 may kill cells through the Bcl2 apoptotic pathways. Western blotting carried out with whole cell extracts prepared from MDA-MB231 cells treated with CTR-20, ABT-737 or in combination of the two for 12 hours. Immunostaining was carried out with antibodies specific for the proteins listed on the right of the blots. GAPDH was used as a loading control. “p-” denotes phosphorylation.

FIG. 41 shows summary of data obtained from screening the NCI-60 cancer panel. Ten μM of CTR-20 was used to examine the drug's efficacy against the NCI-60 cancer cell lines including: six leukemia cell lines, nine non-small cell lung cancer cell lines, seven colorectal cancer cell lines, six CNS cancer cell lines, nine melanoma cell lines, seven ovarian cancer cell lines, seven renal cancer cell lines, two prostate cancer cell lines and six breast cancer cell lines. Screening method was carried out by a sulforhodamine B (SRB) colorimetric assay.

FIG. 42 shows (A) a plot of tumor size (volume in mm³) as a function of days post-treatment “D” in response to drug treatments, alone or in combination with paclitaxel; and (B) exemplary images of representative ATH490 athymic mice engrafted with MDA-MB-231 human metastatic breast cancer cells that were treated with vehicle only (top row) or treated with the drugs paclitaxel (Tax; second row from top); CTR-17 (third row from top); CTR-20 (third row from bottom); paclitaxel and CTR-17 (second row from bottom); and paclitaxel and CTR-20 (bottom row). Numbers in brackets are mg/kg body weight.

FIG. 43 is a plot showing normalized body weight of six-week old ATH40 athymic nude mice treated with vehicle, paclitaxel (Tax), CTR-17, CTR-20, paclitaxel and CTR-17 or paclitaxel and CTR-20 as a function of days (0, 2, 6, 14, 20, 24, 27 or 30) post-drug treatment. The numbers in brackets show drug concentrations in mg/kg body weight. The body weights of ATH490 mice were normalized based on the body weight on day 0 (100%).

FIG. 44 shows plots of the weights of four different organs (liver (A), spleen (B), kidney (C) and lung (D)) of ATH490 mice from different treatments measured at 30-day post-treatment. All values are presented as mean±S.E.M. Each organ weight (%) was normalized with total body weight.

FIG. 45 shows (A) images of livers of ATH490 athymic mice that were treated with sham (top left), 10 mg/kg paclitaxel (top right), 30 mg/kg CTR-17 (middle left), 30 mg/kg CTR-20 (middle right), 5 mg/kg paclitaxel plus 15 mg/kg CTR-17 (bottom left) and 5 mg/kg paclitaxel plus 15 mg/kg CTR-20 (bottom right). White arrows indicate mitotic cells. (B) is a plot showing the number of mitotic cells/mm² as a function of the treatment regimens of (A).

FIG. 46 shows images of spleens of ATH490 athymic mice that were sham-treated (vehicle only; top left) or treated with 10 mg/kg paclitaxel (top right), 30 mg/kg CTR-17 (middle left), 30 mg/kg CTR-20 (middle right), 5 mg/kg paclitaxel plus 15 mg/kg CTR-17 (bottom left) and 5 mg/kg paclitaxel plus 15 mg/kg CTR-20 (bottom right) for 30 days, followed by toxicity analysis after spleen tissues were H & E stained. Arrows indicate the presence of macrophages in the red pulp (RP). Images were taken using a Zeiss EPI-fluorescent microscope (10× objective).

FIG. 47 shows images of kidneys of ATH490 mice that were sham-treated (vehicle only; top left) or treated with 10 mg/kg paclitaxel (top right), 30 mg/kg CTR-17 (middle left), 30 mg/kg CTR-20 (middle right), 5 mg/kg paclitaxel plus 15 mg/kg CTR-17 (bottom left) and 5 mg/kg paclitaxel plus 15 mg/kg CTR-20 (bottom right). At day 30, kidneys were harvested, stained with H&E, and observed under a Zeiss EPI-fluorescent microscope (40× objective). Arrows pointing to the top right image indicate hyaline.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art.

In embodiments of the disclosure, the compounds described herein have at least one asymmetric center. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (e.g. less than 20%, optionally less than 10%, optionally less than 5%, optionally less than 3%) of the corresponding compound having alternate stereochemistry.

In embodiments of the disclosure, the compounds described herein have at least one double bond capable of geometric isomerism; for example, the compound may exist as a cis or a trans isomer. It is to be understood that all such isomers and mixtures thereof in any proportion are encompassed within the scope of the present disclosure. It is to be further understood that while the isomerism of the compounds may be as shown in any given compound listed herein, such compounds may also contain certain amounts (e.g. less than 20%, optionally less than 10%, optionally less than 5%, optionally less than 3%) of the corresponding compound having alternate isomerism.

The term “alkyl” as used herein, whether it is used alone or as part of another group, means straight or branched chain, saturated alkyl groups. The number of carbon atoms that are possible in the referenced alkyl group are indicated by the numerical prefix “Cₙ₁₋ₙ₂”. For example, the term C₁₋₆alkyl means an alkyl group having 1, 2, 3, 4, 5 or 6 carbon atoms.

The term “alkenyl” as used herein, whether it is used alone or as part of another group, means straight or branched chain, unsaturated alkenyl groups. The number of carbon atoms that are possible in the referenced alkenyl group are indicated by the numerical prefix “Cₙ₁₋ₙ₂”. For example, the term C₂₋₆alkenyl means an alkenyl group having 2, 3, 4, 5 or 6 carbon atoms and at least one double bond.

The term “halo” as used herein refers to a halogen atom and includes F, Cl and Br.

The term “haloalkyl” as used herein refers to an alkyl group wherein one or more, including all of the available hydrogen atoms are replaced by a halogen atom. The number of carbon atoms that are possible in the referenced haloalkyl group are indicated by the numerical prefix “Cₙ₁₋ₙ₂”. For example, the term C₁₋₆haloalkyl means a haloalkyl group having 1, 2, 3, 4, 5 or 6 carbon atoms. In an embodiment, the halogen is a fluorine, in which case the haloalkyl is optionally referred to herein as a “fluoroalkyl” group. It is an embodiment that all of the hydrogen atoms are replaced by fluorine atoms. For example, the haloalkyl group can be trifluoromethyl, pentafluoroethyl and the like. It is an embodiment of the present disclosure that the haloalkyl group is trifluoromethyl.

The term “subject” as used herein includes all members of the animal kingdom including mammals, and optionally refers to humans.

The term “pharmaceutically acceptable” means compatible with the treatment of subjects, for example, mammals such as humans.

The term “pharmaceutically acceptable salt” as used herein means an acid addition salt that is compatible with the treatment of subjects.

An “acid addition salt that is compatible with the treatment of subjects” is any non-toxic organic or inorganic salt of any basic compound. Basic compounds that form an acid addition salt include, for example, compounds comprising an amine group susceptible to protonation. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Such salts may exist in a hydrated, solvated or substantially anhydrous form. In general, acid addition salts are more soluble in water and various hydrophilic organic solvents, and generally demonstrate higher melting points in comparison to their free base forms. The selection of a suitable salt can be made by a person skilled in the art. The formation of a desired acid addition salt is, for example, achieved using standard techniques. For example, the neutral compound is treated with the desired acid in a suitable solvent and the salt which is thereby formed then isolated by filtration, extraction and/or any other suitable method.

The term “solvates” as used herein in reference to a compound refers to complexes formed between the compound and a solvent from which the compound is precipitated or in which the compound is made. Accordingly, the term “solvate” as used herein means a compound, or a salt of a compound, wherein molecules of a suitable solvent are incorporated in the crystal lattice. Examples of suitable solvents are ethanol, water and the like. When water is the solvent, the molecule is optionally referred to as a “hydrate”. The formation of solvates will vary depending on the compound and the solvate. In general, solvates are formed by dissolving the compound in an appropriate solvent and isolating the solvate by cooling or using an antisolvent. The solvate is typically dried or azeotroped under ambient conditions. The selection of suitable conditions to form a particular solvate can be made by a person skilled in the art.

The term “prodrug” as used herein in reference to a compound refers to a derivative of the compound that reacts under biological conditions to provide the compound. In an embodiment, the prodrug comprises a conventional ester formed with an available amino group. For example, an available amino group is acylated using an activated acid in the presence of a base, and optionally, in an inert solvent (e.g. an acid chloride in pyridine). Some common esters which have been used as prodrugs are phenyl esters, aliphatic (C₁-C₂₄) esters, acyloxymethyl esters, carbamates and amino acid esters.

The one or more compounds of the application are, for example, administered to the subject or used in an “effective amount”.

As used herein, the term “effective amount” and the like means an amount effective, at dosages and for periods of time necessary to achieve a desired result. For example, in the context of treating cancer, an effective amount of the one or more compounds of the disclosure is an amount that, for example, reduces the cancer compared to the cancer without administration of the one or more compounds of the disclosure. Effective amounts may vary according to factors such as the disease state, age, sex, weight and/or species of the subject. The amount of a given compound that will correspond to such an amount will vary depending upon various factors, such as the given compound, the pharmaceutical formulation, the route of administration, the type of condition, disease or disorder being treated, the identity of the subject being treated, and the like, but can nevertheless be routinely determined by one skilled in the art.

### II. Compounds and Methods of Preparation Thereof

The microtubule is the target for several different anticancer therapeutic agents including colchicine and paclitaxel. However, the use of colchicine as an anticancer agent has not been approved, for example, by the U.S. Food and Drug Administration due mainly to its inherent toxicity. Accordingly, it is an object of the studies of the present disclosure to develop an anticancer agent targeting the colchicine-binding site on microtubule with minimum toxicity. Several chalcone derivatives were synthesized and examined. Data from the present study with three human breast cancer cell lines (MDA-MB-468, MDA-MB-231 and MCF-7) and two matching non-cancer breast cell lines (184B5 and MCF-10A) showed that CTR-17 and CTR-20 are useful anticancer leads. The study was also expanded to several other cancer cell lines including K562 (chronic myelogenous leukemia [CML] cell line), HeLa (cervical cancer), U87MG (brain cancer), T98G (temozolomide-resistant brain cancer), NCI-H1975 (lung cancer), A549 (lung cancer), RPMI-8226 (multiple myeloma), RPMI-8226-BR (Bortezomib-resistant RPMI-8226 cell line for the compound CTR-20 only), KB-3-1 (cervical cancer), KB-C-2 (colchicine-resistant and paclitaxel-resistant KB-3-1 cell line), ANBL6-BR (bortezomib-resistant multiple myeloma for the compound CTR-20 only), H69 (small cell lung cancer) and H69AR (multidrug-resistant small cell lung epithelial cancer). It was found that: (a) CTR-17 and CTR-20 preferentially kill cancer over non-cancer cells, up to 26 times (CTR-17, on MDA-MB-468 versus MCF-10A) and 24 times (CTR-20, on HeLa versus MCF-10A); (b) CTR-17 and CTR-20 induce a prolonged cell cycle arrest at the spindle checkpoint step in a cancer cell-specific manner, eventually leading to cancer cell death by apoptosis; (c) CTR-17 and CTR-20 inhibit tubulin polymerisation; (d) the dissociation constants of CTR-17 and CTR-20 are 4.58±0.95 μM and 5.09±0.49 μM, respectively; (e) the microtubule binding sites of both CTR-17 and CTR-20 almost overlap with that of colchicine; (f) unlike colchicine, the effects of the CTR-17 and CTR-20 compounds are reversible; (g) data from in silico molecular docking studies suggests, while not wishing to be limited by theory, that CTR-17, CTR-20 and colchicine, respectively, form one, two and three hydrogen bonds (H-bonds) with amino acid residues of tubulin, in addition to forming strong Van der Waals interactions; (h) CTR-17 and CTR-20 kill MDR1-overexpressing and MRP1-overexpressing multidrug-resistant cancer cells (which are also paclitaxel/colchicine-resistant); (i) CTR-20 killed bortezomib-resistant multiple myeloma cells (IC₅₀ values of RPMI-8226-BR and ANBL6-BR were 0.28±0.03 and 0.76±0.28 μM, respectively); (j) the combinational treatment of MDR1 overexpressing KB-C-2 cells with paclitaxel and CTR-17 or CTR-20 showed synergistic effects; (k) data from in vitro and animal studies shows that both CTR-17 and CTR-20 are useful as antitumor agents; and (l) data from studies with engrafted mice showed that the combination of ½ doses of CTR-20 and paclitaxel is more efficient than the full dose of either compound alone, without causing any notable ill-effects. Together, the data indicates that CTR compounds, for example, CTR-20, in one embodiment, are useful anticancer agents that can, for example, kill many different cancer cells including colchicine/paclitaxel-resistant, bortezomib-resistant, and multidrug-resistant tumor cells with no notable ill-effects which were observed in the present studies on non-cancer cells and normal mouse organs. Further, (m) Studies carried out with 16 compounds (CTR-21 to CTR-40) shows that they kill tumor cells with IC₅₀ values ranging from 5.34 nM (CTR-21 against RPMI-8226) to 2.69 μM (CTR-27 against MDA-MB231); (n) further studies showed that CTR-21 and CTR-32 are potent against tumor cells, MDA-MB231, MCF-7, HeLa and RPMI-8226, as their IC₅₀ values are in the nonomolar range; (o) a study with isogenic breast and breast cancer cell lines showed that CTR-17, CTR-20, CTR-21 and CTR-32 preferentially kill fully malignant cells over pre-cancer or non-cancer cells; (p) CTR-21 and CTR-32 are microtubule polymerization inhibitors; (q) similar to CTR-20, CTR-21 and CTR-32 are reversible mitotic inhibitors; (r) the combination of CTR-20 and ABT-737, an inhibitor of the Bcl2 anti-apoptotic family proteins, is synergistic against MDA-MB231 triple-negative metastatic breast cancer, as the combinational index is 0.07-0.10; (s) CTR-20, CTR-21 and CTR-32 bortezomib-resistant RPMI-8226BTZR cells; (t) CTR-20, CTR-21 and CTR-32 kill the multidrug- and paclitaxel-resistant MDA-MB231TaxR cells, and also when combined with paclitaxel and CTR-20; (u) CTR-20 kills NCI-60 cancer cell lines including: six leukemia cell lines, nine non-small cell lung cancer cell lines, seven colorectal cancer cell lines, six CNS cancer cell lines, nine melanoma cell lines, seven ovarian cancer cell lines, seven renal cancer cell lines, two prostate cancer cell lines and six breast cancer cell lines.

Accordingly, the present disclosure includes a compound of Formula I:

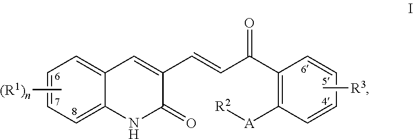

wherein

A is O or S;

n is 0, 1, 2 or 3;

when n is 1, R¹ is halo, C₁₋₆alkyl, C₂₋₆alkenyl or —X—C₁₋₆alkyl;

when n is 2 or 3, each R¹ is independently halo, C₁₋₆alkyl, C₂₋₆alkenyl or —X—C₁₋₆alkyl; or two R¹ together form a methylenedioxy group that is attached to two adjacent ring carbon atoms;

R² is C₁₋₆alkyl or C₁₋₆haloalkyl;

R³ is absent or is halo, —X—C₁₋₆alkyl or —X—C₁₋₆haloalkyl; and

each X is independently O or S,

or a pharmaceutically acceptable salt, solvate and/or prodrug thereof.

In an embodiment, A is O. In another embodiment, X is S.

In an embodiment, X is O. In another embodiment, X is S.

In an embodiment, R³ is absent. In another embodiment, R³ is F, —X—C₁₋₄alkyl or —X—C₁₋₄haloalkyl. In a further embodiment, R³ is F, —O—C₁₋₄alkyl or —O—C₁₋₄haloalkyl. It is an embodiment that R³ is F, —OCH₃ or —OCF₃. In another embodiment, R³ is 4′-OCH₃, 5′-OCH₃, 6′-OCH₃, 4′-OCF₃, 4′-F or 5′-F.

In an embodiment, R² is C₁₋₄alkyl or C₁₋₄haloalkyl. In another embodiment, R² is CH₃ or CF₃. In a further embodiment, R² is CH₃. It is an embodiment of the present disclosure that R² is CF₃.

In an embodiment, n is 0, 1 or 2. In another embodiment, n is 0 or 1. In a further embodiment, n is 0. It is an embodiment that n is 1. In another embodiment, n is 2. In a further embodiment, n is 3.

In an embodiment, n is 1 and R¹ is halo, C₁₋₄alkyl, C₂₋₄alkenyl or —X—C₁₋₄alkyl. In another embodiment, n is 1 and R¹ is CH₃ or OCH₃. In a further embodiment, n is 1 and R¹ is 6-OCH₃, 7-OCH₃, 8-OCH₃, 6-OC₂H₅, 6-SCH₃, 7-SCH₃, 6-CH₃, 6-C₂H₅, 6-F, 6-Cl, 6-Br, 7-F, 7-Cl or 7-Br. It is an embodiment that n is 1 and R¹ is 6-CH₃, 6-OCH₃ or 7-OCH₃. In another embodiment, n is 1 and R¹ is 6-OCH₃.

In an embodiment, n is 2 and each R¹ is independently halo, C₁₋₄alkyl, C₂₋₄alkenyl or —X—C₁₋₄alkyl; or two R¹ together form a methylenedioxy group that is attached to two adjacent ring carbon atoms. In another embodiment, each R¹ is independently CH₃ or OCH₃; or two R¹ together form a methylenedioxy group that is attached to two adjacent ring carbon atoms. In a further embodiment, n is 2 and R¹ is 6,7-diCH₃, 6,7-diOCH₃ or 6,7-O—CH₂—O—. It is an embodiment that n is 2 and R¹ is 6,7-diCH₃ or 6,7-diOCH₃.

In an embodiment, n is 3 and each R¹ is independently halo, C₁₋₄alkyl, C₂₋₄alkenyl or —X—C₁₋₄alkyl. In another embodiment, n is 3 and each R¹ is independently CH₃ or OCH₃. In a further embodiment, n is 3 and R¹ is 5,6,7-triOCH₃.

In an embodiment, the compound is selected from:

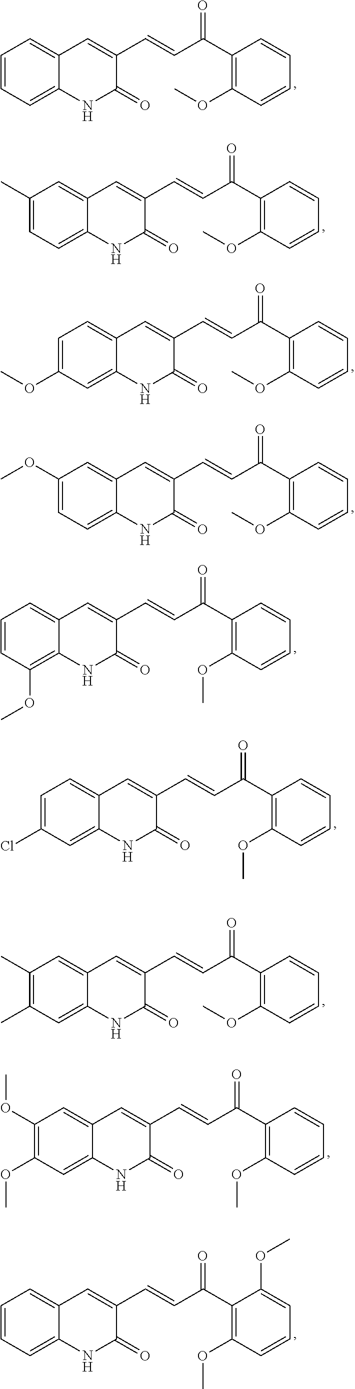

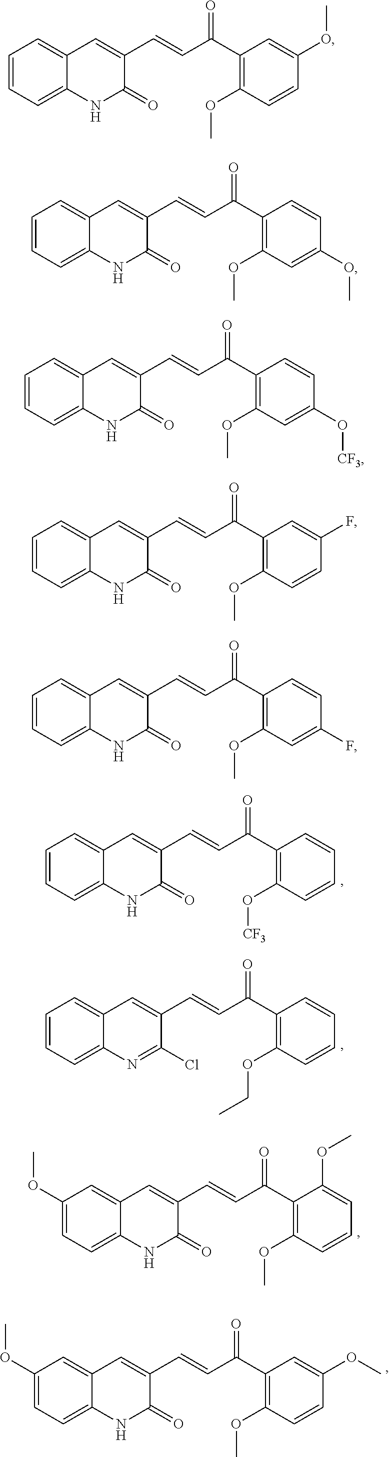

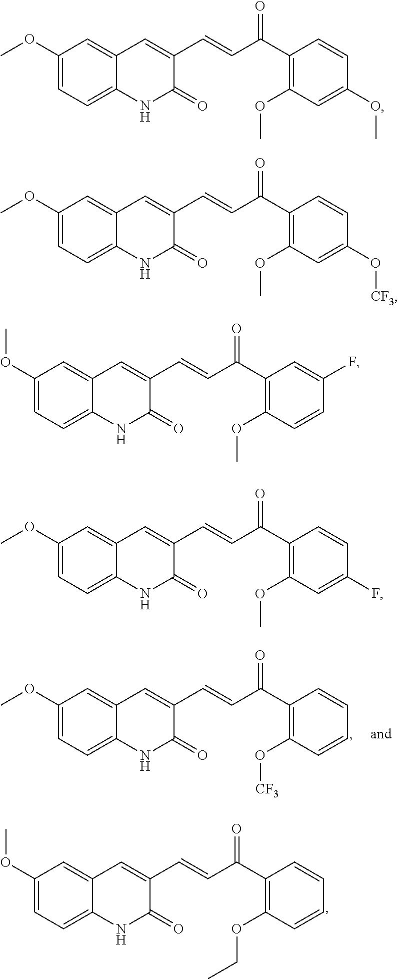

or a pharmaceutically acceptable salt, solvate and/or prodrug thereof.

In another embodiment, the compound is:

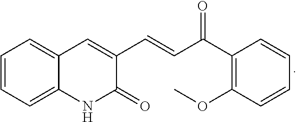 [SMILES: COc1ccccc1C(=O)/C=C/c1cc2ccccc2[nH]c1=O]

In a further embodiment, the compound is:

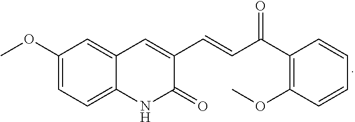 [SMILES: COc1ccc2[nH]c(=O)c(/C=C/C(=O)c3ccccc3OC)cc2c1]

In an embodiment, A is O and the compounds of the disclosure are prepared, for example, by the reaction sequences shown in general synthetic scheme 1. A person skilled in the art could readily adapt such a synthesis to prepare the corresponding compounds wherein A is S.

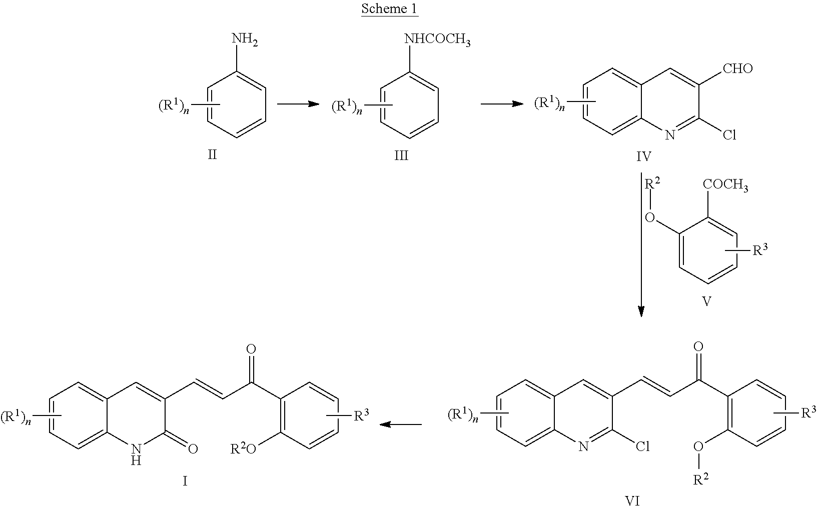

In an embodiment of the present disclosure, a compound of Formula I is prepared by a method comprising treating an acetanilide of Formula III with DMF and POCl₃ under Vilsmeier Haack conditions to obtain a 2-chloroquinoline 3-carboxaldehyde of Formula IV; performing Claisen-Schmidt condensation of the 2-chloroquinoline 3-carboxaldehyde of Formula IV with a substituted acetophenone of Formula V under basic conditions (for example, a catalytic amount of sodium methoxide or NaOH) to obtain a 3-(2-chloroquinolin-3-yl)-1-phenylprop-2-en-1-one of Formula VI; and reacting the 3-(2-chloroquinolin-3-yl)-1-phenylprop-2-en-1-one of Formula VI under conditions so that it undergoes O-nucleophilic substitution at the 2-chloro group (for example, treatment with aqueous glacial acetic acid under reflux) to provide the quinolone chalcone of Formula I. The acetanilide of Formula III is optionally commercially available. Alternatively, the acetanilide of Formula III is prepared from the corresponding anilines according to standard procedures (Vogel et al., 1996). In the compounds of Formulae I to VI, R¹, R², R³ and n are as defined herein.

### III. Compositions

The present disclosure also includes a composition comprising one or more compounds of the present disclosure and a carrier. The compounds of the disclosure are optionally formulated into pharmaceutical compositions for administration to subjects or use in a biologically compatible form suitable for administration or use in vivo. Accordingly, the present disclosure further includes a pharmaceutical composition comprising one or more compounds of the present disclosure and a pharmaceutically acceptable carrier.

The compounds of the disclosure can be administered to a subject or used in a variety of forms depending on the selected route of administration or use, as will be understood by those skilled in the art. In an embodiment, the one or more compounds of the disclosure are administered to the subject, or used, by oral (including buccal) or parenteral (including intravenous, intraperitoneal, subcutaneous, intramuscular, transepithelial, nasal, intrapulmonary, intrathecal, rectal, topical, patch, pump and transdermal) administration or use and the compound(s) formulated accordingly. For example, the compounds of the disclosure are administered or used in an injection, in a spray, in a tablet/caplet, in a powder, topically, in a gel, in drops, by a patch, by an implant, by a slow release pump or by any other suitable method of administration or use, the selection of which can be made by a person skilled in the art.

In an embodiment, the one or more compounds of the present disclosure are orally administered or used, for example, with an inert diluent or with an assimilable edible carrier, or enclosed in hard or soft shell gelatin capsules, or compressed into tablets, or incorporated directly with the food of the diet. In an embodiment, for oral therapeutic administration or use, the one or more compounds of the disclosure are incorporated with excipient and administered or used in the form of ingestible tablets, buccal tablets, troches, capsules, elixirs, suspensions, syrups, wafers, and the like. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In an embodiment, liposomes are formed from a variety of phospholipids, such as cholesterol, stearylamine and/or phosphatidylcholines.

In another embodiment of the present disclosure, the one or more compounds of the present disclosure are administered or used parenterally. Solutions of the one or more compounds of the present disclosure are, for example, prepared in water optionally mixed with a surfactant such as hydroxypropylcellulose. In a further example, dispersions are prepared in glycerol, liquid polyethylene glycols, DMSO and mixtures thereof with or without alcohol, and in oils. Pharmaceutical forms suitable for injectable administration or use include sterile aqueous solutions or dispersions and sterile powders for the extemporaneous preparation of sterile injectable solutions or dispersions. A person skilled in the art would know how to prepare suitable formulations.

### IV. Methods of Treatment and Uses

The compounds of the present disclosure are new therefore the present disclosure includes all uses for compounds of the present disclosure, including use in therapeutic methods, diagnostic assays, and as research tools whether alone or in combination with another active pharmaceutical ingredient.

Together, the data from the present studies indicates that CTR compounds, for example, CTR-20, are useful anticancer agents that can, for example, kill many different cancer cells including colchicine/paclitaxel-resistant, bortezomib-resistant, and multidrug-resistant tumor cells with no notable ill-effects observed on non-cancer cells and normal mouse organs.

Therefore, in an embodiment, the compounds of the present disclosure are useful as medicaments. Accordingly, the present disclosure includes one or more compounds of the present disclosure for use as a medicament.

The present disclosure also includes a method of treating cancer comprising administering one or more compounds of the disclosure to a subject in need thereof. The present disclosure also includes a use of one or more compounds of the disclosure for treating cancer in a subject; a use of one or more compounds of the disclosure for preparation of a medicament for treating cancer in a subject; and one or more compounds of the disclosure for use to treat cancer in a subject.

In an embodiment, the cancer is breast cancer, leukemia, cervical cancer, brain cancer, lung cancer, bladder cancer, kidney cancer, colorectal cancer, CNS cancer, melanomas, ovarian cancer, prostate cancer, multiple myeloma or other blood cancers. In another embodiment, the cancer comprises colchicine-resistant, paclitaxel-resistant, bortezomib-resistant, vinblastine-resistant and/or multidrug-resistant tumor cells.

Treatment methods or uses comprise administering to a subject or use of an effective amount of one or more compounds of the disclosure, optionally consisting of a single administration or use, or alternatively comprising a series of administrations or uses. For example, the compounds of the disclosure are administered or used at least once a week. However, in another embodiment, the compounds are administered to the subject or used from one time per three weeks, or one time per week to once daily for a given treatment or use. In another embodiment, the compounds are administered or used 2, 3, 4, 5 or 6 times daily. The length of the treatment period or use depends on a variety of factors, such as the severity of the cancer, the age of the subject, the concentration of the one or more compounds in a formulation, the activity of the compounds of the present disclosure, and/or a combination thereof. It will also be appreciated that the effective amount of a compound used for the treatment or use may increase or decrease over the course of a particular treatment regime or use. Changes in dosage may result and become apparent by standard diagnostic assays known in the art. In some instances, chronic administration or use is required. For example, the one or more compounds of the present disclosure are administered or used in an amount and for duration sufficient to treat the subject.

The extent and/or undesirable clinical manifestations of cancer are optionally lessened (palliated) and/or the time course of the progression is slowed or lengthened, as compared to not treating the cancer.

The one or more compounds of the disclosure may be administered or used alone or in combination with other therapeutic agents useful for treating cancer; (optionally referred to herein as “anticancer agents”). When administered or used in combination with other known therapeutic agents, it is an embodiment that the one or more compounds of the disclosure are administered or used contemporaneously with those therapeutic agents. As used herein the term “contemporaneous” in reference to administration of two substances to a subject or use means providing each of the two substances so that they are both biologically active in the individual at the same time. The exact details of the administration or use will depend on the pharmacokinetics of the two substances in the presence of each other, and can include administering or using the two substances within a few hours of each other, or even administering or using one substance within 24 hours of administration or use of the other, if the pharmacokinetics are suitable. Design of suitable dosing regimens is routine for one skilled in the art. In particular embodiments, two substances will be administered or used substantially simultaneously, i.e., within minutes of each other, or in a single composition that contains both substances. It is a further embodiment that a combination of the two substances is administered to a subject or used in a non-contemporaneous fashion.

In an embodiment, the other agents are selected from the group consisting of mitotic inhibitors (for example, paclitaxel); bcl2 inhibitors (for example, ABT-737); proteasome inhibitors (for example, bortezomib or calfilzomib); signal transduction inhibitors (for example, gefitinib, erlotinib, dasatinib, imatinib or sunitinib); inhibitors of DNA repair (for example, iniparib, temozolomide or doxorubicin); and alkylating agents (for example, cyclophosphamide). In another embodiment, the other anticancer agent is paclitaxel.

The dosage of compounds of the disclosure can vary depending on many factors such as the pharmacodynamic properties of the compound, the mode of administration or use, the age, health and weight of the subject, the nature and extent of the symptoms of the cancer, the frequency of the treatment or use and the type of concurrent treatment or use, if any, and the clearance rate of the compound in the subject. One of skill in the art can determine the appropriate dosage based on the above factors. In an embodiment, the compounds of the disclosure are administered or used initially in a suitable dosage that is optionally adjusted as required, depending on the clinical response. As a representative example, oral dosages of one or more compounds of the disclosure will range from less than 1 mg per day to 1000 mg per day for a human adult or an animal. In an embodiment of the present disclosure, the pharmaceutical compositions are formulated for oral administration or use and the compounds are, for example in the form of tablets containing 0.001, 0.01, 0.1, 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. In an embodiment, the compounds of the disclosure are administered or used in a single daily dose or the total daily dose may be divided into two, three or four daily doses.

In the studies of the present disclosure, the combinational treatment of MDR1 overexpressing KB-C-2 or MDA-MB231TaxR cells with paclitaxel and CTR-17, CTR-20, CTR-21 or CTR-32 showed synergistic effects and data from studies with engrafted mice showed that the combination of ½ doses of CTR-20 and paclitaxel is more efficient than the full dose of either compound alone, without causing any notable ill-effects.

Accordingly, in embodiments wherein the one or more compounds of the present disclosure are administered or used in combination with one or more other anticancer agents, the dosage of the one or more compounds of the present disclosure is optionally less than the dosage of the one or more compounds of the present disclosure when administered or used alone. In another embodiment, the dosage of the one or more compounds of the present disclosure is one half the dosage of the one or more compounds of the present disclosure when administered or used alone.

In embodiments wherein the one or more compounds of the present disclosure are administered or used in combination with one or more other anticancer agents, the dosage of the other anticancer agent is optionally less than the dosage of the other anticancer agent when administered or used alone. In another embodiment, the dosage of the other anticancer agent is one half the dosage of the other anticancer agent when administered or used alone.

The following non-limiting examples are illustrative of the present disclosure:

### EXAMPLES

### Example 1: Synthesis and Characterization of Compounds

I. Materials and Methods

The chemicals and solvents used were commercially available and were of reagent grade. Melting points were determined in open glass capillaries on a Veego digital melting point apparatus and were uncorrected. The infrared (IR) spectra of the compounds were recorded on Schimadzu FT-IR 8400S infrared spectrophotometer using an ATR accessory. ¹H NMR spectra were recorded on a Bruker Avance II 400 spectrometer, using DMSO-d₆ as solvent and TMS as internal standard. Mass spectral analysis was carried out using Applied Biosystem QTRAP 3200 MS/MS system in ESI mode. Reactions were monitored by TLC using pre-coated silica gel aluminum plates (Kieselgel 60, 254, E. Merck, Germany); zones were detected visually under ultraviolet irradiation.

II. General Synthetic Procedures

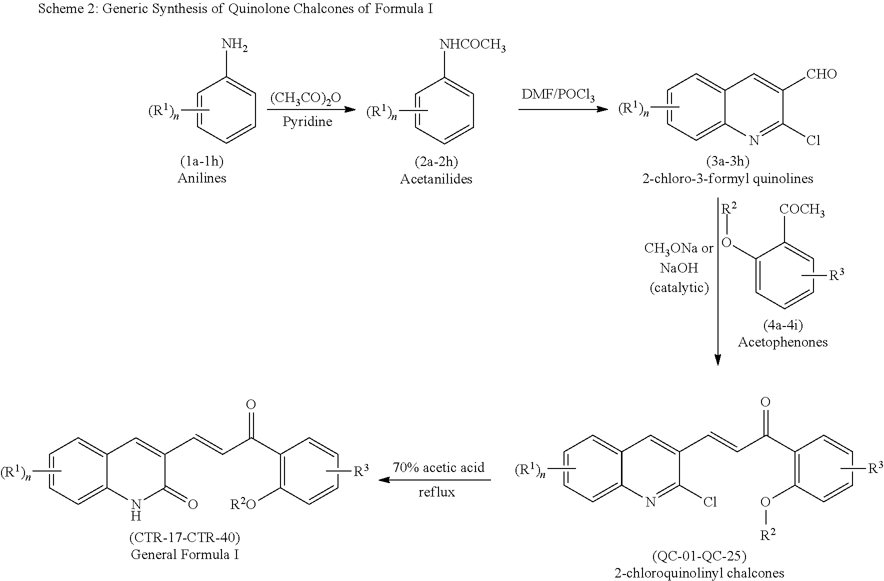

The synthesis of the quinolone chalcones of Formula I was carried out according to the reaction sequence illustrated in Scheme 2, wherein n was 0, 1 or 2, R¹ was 6-OCH₃, 7-OCH₃, 8-OCH₃, 6,7-diOCH₃, 6-CH₃, 6,7-diCH₃, 6-Cl, 6-Br or 7-Cl, R² was CH₃, C₂H₅ or CF₃ and R³ was absent or was OCH₃, OCF₃ or F, as appropriate for the compounds described herein. The acetanilides (2a-2h) utilized in the synthetic route were either commercially available or synthesized from corresponding anilines (1a-1h) according to standard procedures (Vogel et al., 1996). The acetanilides (2a-2h) were then treated with DMF and POCl₃ under Vilsmeier Haack conditions to give 2-chloroquinoline 3-carboxaldehydes (3a-3h) (Meth-Cohn et al., 1981). Then, Claisen-Schmidt condensation of the 2-chloroquinoline-3-carbaldehydes (3a-3h) with the desired substituted acetophenones (4a-4i) under basic conditions (a catalytic amount of sodium methoxide or NaOH) furnished 3-(2-chloroquinolin-3-yl)-1-phenylprop-2-en-1-ones (QC-01-QC-25) in high yields (Dominguez et al., 2001; Li et al., 1995). The 3-(2-chloroquinolin-3-yl)-1-phenylprop-2-en-1-ones upon treatment with aqueous glacial acetic acid under reflux conditions then underwent 0-nucleophilic substitution at the 2-chloro group of the quinoline ring to give the corresponding quinolone chalcones of Formula I (CTR-17-CTR-40). The structural identity of the synthesized compounds was established on the basis of their infrared (IR) spectroscopic, ¹H NMR and mass spectral data.

### (a) General Procedure for the Synthesis of 2 chloro-3-formyl quinolines (3a-3h) (Meth-Cohn et al., 1981)

Acetanilide (2a)/substituted acetanilides (2b-2h) (0.05 mol) were dissolved in 9.6 ml of dimethyl formamide (0.125 mol) and to this solution, 32 ml of phosphorus oxychloride (0.35 mol) was added gradually at 0° C. The reaction mixture was taken in a round bottom flask (RBF) equipped with a reflux condenser fitted with a drying tube and was heated for 4-16 hours on oil bath at 75-80° C. The solution was then cooled to room temperature and subsequently poured onto 100 ml of ice water. The precipitate formed was collected by filtration and recrystallized from ethyl acetate.

### (b) General Procedure for the Synthesis of 2-chloroquinolinyl chalcones (QC-01-QC-25) (Dominguez et al., 2001; Li et al., 1995)

A mixture of 2-chloro-3-formyl quinolines (3a-3h) (1 mmol), the respective acetophenones (4a-4i) (1 mmol) and a base (sodium methoxide (catalytic) or sodium hydroxide (one pellet)) in methanol (4 ml) was stirred at room temperature for 6-24 hr. The resulting precipitate was collected by filtration, washed with water and recrystallized from DMF-H₂O or EtOH-H₂O.

### (c) General Procedure for the Synthesis of 3-(3-oxo-3-phenylprop-1-enyl)quinolin-2(1H)-ones of Formula I (CTR-17 to CTR-40)

A suspension of the 3-(2-chloroquinolin-3-yl)-1-phenylprop-2-en-1-ones (QC-01-QC-25) (0.001 mol) in 70% acetic acid (10 ml) was heated under reflux for 4-6 hr. Upon completion of the reaction (as indicated by a single spot in a TLC), the reaction mixture was cooled to ambient temperature and the solid product precipitated out was filtered. The filtered product was washed with water, dried and recrystallized in methanol or DMF/water.

III. Synthesis of Representative Compounds of the Disclosure

### (a) Synthesis of (E)-3-(3-(2-Methoxyphenyl)-3-oxoprop-1-enyl)quinolin-2(1H)-one (CTR-17)

### 2-Chloroquinoline-3-carbaldehyde (3a)

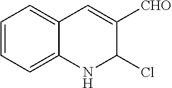 [SMILES: O=CC1=Cc2ccccc2NC1Cl]

The title compound was prepared from acetanilide (2a) following the protocol described above under subsection II(a) in the general procedure for the synthesis of 2 chloro-3-formyl quinolines.

Yield 72%; M. P. 148-150° C. (Lit. 149° C.) (Srivastava and Singh, 2005); FT-IR (ATR) υ (cm⁻¹): 3044 (Aromatic C—H), 2870 (aldehyde C—H), 1684 (C═O), 1574 (C═N), 1045 (C—Cl); ¹H NMR (400 MHz, Chloroform-d): δ 10.57 (s, 1H), 8.77 (s, 1H), 8.08 (d, J=8.5 Hz, 1H), 7.99 (d, J=8.1 Hz, 1H), 7.90 (t, J=7.7 Hz, 1H), 7.66 (t, J=8.0 Hz, 1H); MS-API: [M+H]⁺ 192 (calculated 191.01).

### (E)-3-(2-Chloroquinolin-3-yl)-1-(2-methoxyphenyl)prop-2-en-1-one (QC-01)

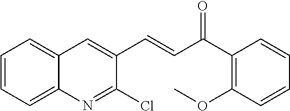 [SMILES: COc1ccccc1C(=O)/C=C/c1cc2ccccc2nc1Cl]

The title compound was prepared by Claisen-Schmidt condensation of 2-chloroquinoline-3-carbaldehyde (3a) with 2-methoxy acetophenone (4a) following the protocol described above under subsection II(b) in the general procedure for the synthesis of 2-chloroquinolinyl chalcones.

Yield 62%; M.P. 113-115° C.; FT-IR (ATR) υ (cm⁻¹): 3067 (Aromatic C—H), 1653 (C═O), 1603 (C═C), 1242 (C—O—C), 1045 (C—Cl); ¹H NMR (400 MHz, Chloroform-d): δ 8.43 (s, 1H), 8.08-8.00 (m, 2H), 7.87 (d, J=8.3 Hz, 1H), 7.77 (t, J=7.8 Hz, 1H), 7.68 (dd, J=7.5, 1.8 Hz, 1H), 7.63-7.56 (m, 1H), 7.55-7.45 (m, 2H), 7.11-7.01 (m, 2H), 3.94 (s, 3H); MS-API: [M+H]⁺ 324.1 (calculated 323.07).

### (E)-3-(3-(2-Methoxyphenyl)-3-oxoprop-1-enyl)quinolin-2(1H)-one (CTR-17)

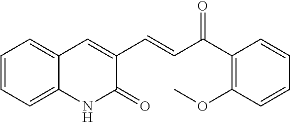 [SMILES: COc1ccccc1C(=O)/C=C/c1cc2ccccc2[nH]c1=O]

The title compound was prepared by refluxing 3-(2-Chloroquinolin-3-yl)-1-(2-methoxyphenyl)prop-2-en-1-one (QC-01) in aqueous acetic acid (70%) following the protocol described above under subsection II(c) in the general procedure for the synthesis of 3-(3-oxo-3-phenylprop-1-enyl)quinolin-2(1H)-ones.

Yield 81%; M.P. 256-258° C.; FT-IR (KBr) υ (cm⁻¹): 3153 (NH), 1656 (C═O), 1586, 1557 (C═C), 1240, 1020 (C—O—C); ¹H NMR (400 MHz, DMSO-d₆): (12.05 (s, 1H), 8.47 (s, 1H), 7.86 (d, J=16.0 Hz, 1H), 7.73 (d, J=7.9 Hz, 1H), 7.60-7.44 (m, 4H), 7.34 (d, J=8.3 Hz, 1H), 7.26-7.19 (m, 2H), 7.08 (td, J=7.4, 0.9 Hz, 1H), 3.87 (s, 3H); MS-API: [M+H]⁺ 306.1 (calculated 305.1).

### (b) Synthesis of (E)-3-(3-(2-Methoxyphenyl)-3-oxoprop-1-enyl)-6-methylquinolin-2(1H)-one (CTR-18)

### 2-Chloro-6-methylquinoline-3-carbaldehyde (3b)

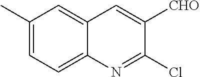

The title compound was prepared from N-p-tolylacetamide (2b) following the protocol described above under subsection II(a) in the general procedure for the synthesis of 2 chloro-3-formyl quinolines.

Yield 75%; M.P. 122-123° C. (Lit. 123° C.) (Srivastava and Singh, 2005); FT-IR (ATR) υ (cm⁻¹): 3051 (Aromatic C—H), 2873 (aldehyde C—H), 1686 (C═O), 1576 (C═N), 1055 (C—Cl); ¹H NMR (400 MHz, Chloroform-d): δ 10.55 (s, 1H), 8.66 (s, 1H), 7.96 (d, J=8.5 Hz, 1H), 7.79-7.63 (m, 2H), 2.57 (s, 3H); MS-API: [M+H]⁺ 206.02 (calculated 205.03).

### (E)-3-(2-Chloro-6-methylquinolin-3-yl)-1-(2-methoxyphenyl)prop-2-en-1-one (QC-02)

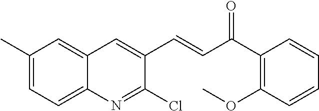 [SMILES: COc1ccccc1C(=O)/C=C/c1cc2cc(C)ccc2nc1Cl]

The title compound was prepared by Claisen-Schmidt condensation of 2-Chloro-6-methylquinoline-3-carbaldehyde (3b) with 2-methoxy acetophenone (4a) following the protocol described above under subsection II(b) in the general procedure for the synthesis of 2-chloroquinolinyl chalcones.

Yield 67%; M.P. 132-135° C.; FT-IR (ATR) υ (cm⁻¹): 3071 (Aromatic C—H), 2833 (Aliphatic C—H), 1641 (C═O), 1597 (C═C), 1240, 1026 (C—O—C), 1047 (C—Cl); ¹H NMR (400 MHz, Chloroform-d): δ 8.34 (s, 1H), 8.02 (d, J=15.9 Hz, 1H), 7.91 (d, J=8.6 Hz, 1H), 7.68 (dd, J=7.6, 1.8 Hz, 1H), 7.65-7.55 (m, 2H), 7.54-7.41 (m, 2H), 7.18-6.90 (m, 2H), 3.93 (s, 3H), 2.55 (s, 3H); MS-API: [M+H]⁺ 338.1 (calculated 337.09).

### (E)-3-(3-(2-Methoxyphenyl)-3-oxoprop-1-enyl)-6-methylquinolin-2(1H)-one (CTR-18)

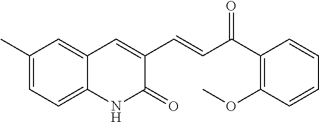 [SMILES: COc1ccccc1C(=O)/C=C/c1cc2cc(C)ccc2[nH]c1=O]

The title compound was prepared by refluxing 3-(2-Chloro-6-methylquinolin-3-yl)-1-(2-methoxyphenyl)prop-2-en-1-one (QC-02) in aqueous acetic acid (70%) following the protocol described above under subsection II(c) in the general procedure for the synthesis of 3-(3-oxo-3-phenylprop-1-enyl)quinolin-2(1H)-ones.

Yield 86%; M.P. 222-224° C.; FT-IR (KBr) υ (cm⁻¹): 3145 (NH), 1654 (C═O), 1584, 1558 (C═C), 1241, 1019 (C—O—C); ¹H NMR (400 MHz, DMSO-d₆): δ 11.88 (s, 1H), 8.15 (s, 1H), 7.89 (d, J=15.9 Hz, 1H), 7.58 (d, J=15.9 Hz, 1H), 7.50 (t, J=7.5 Hz, 2H), 7.44 (s, 1H), 7.32 (d, J=8.5 Hz, 1H), 7.26 (d, J=8.4 Hz, 1H), 7.10 (d, J=8.3 Hz, 1H), 7.04 (t, J=7.4 Hz, 1H), 3.90 (s, 3H), 2.39 (s, 3H), MS-API: [M+H]⁺ 320.1 (calculated 319.12).

### (c) Synthesis of (E)-7-Methoxy-3-(3-(2-methoxyphenyl)-3-oxoprop-1-enyl) quinolin-2(1H)-one (CTR-19)

### 2-Chloro-7-methoxyquinoline-3-carbaldehyde (3c)

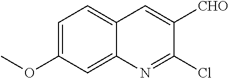

The title compound was prepared from N-(3-methoxyphenyl)acetamide (2c) following the protocol described above under subsection II(a) in the general procedure for the synthesis of 2 chloro-3-formyl quinolines.

Yield 78%; M.P. 195-196° C. (Lit. 196° C.) (Srivastava and Singh, 2005); FT-IR (ATR) υ (cm⁻¹): 3053 (Aromatic C—H), 2879 (aldehyde C—H), 1688 (C═O), 1583 (C═N), 1240, 1043 (C—O—C), 1051 (C—Cl); ¹H NMR (400 MHz, Chloroform-d): δ 10.51 (s, 1H), 8.66 (s, 1H), 7.85 (d, J=9.0 Hz, 1H), 7.38 (s, 1H), 7.27 (dd, J=9.0, 2.5 Hz, 1H), 3.98 (s, 3H); MS-API: [M+H]⁺ 222.02 (calculated 221.02).

### 3-(2-Chloro-7-methoxyquinolin-3-yl)-1-(2-methoxyphenyl)prop-2-en-1-one (QC-03)

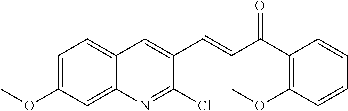 [SMILES: COc1ccc2cc(/C=C/C(=O)c3ccccc3OC)c(Cl)nc2c1]

The title compound was prepared by Claisen-Schmidt condensation of 2-Chloro-7-methoxyquinoline-3-carbaldehyde (3c) with 2-methoxy acetophenone (4a) following the protocol described above under subsection II(b) in the general procedure for the synthesis of 2-chloroquinolinyl chalcones.

Yield 63%; M.P. 178-180° C.; FT-IR (ATR) υ (cm⁻¹): 3073 (Aromatic C—H), 2837 (Aliphatic C—H), 1666 (C═O), 1595 (C═C), 1227, 1020 (C—O—C), 1055 (C—Cl); ¹H NMR (400 MHz, Chloroform-d): δ 8.35 (s, 1H), 8.02 (d, J=15.9 Hz, 1H), 7.74 (d, J=9.0 Hz, 1H), 7.66 (dd, J=7.6, 1.9 Hz, 1H), 7.51 (ddd, J=8.9, 7.5, 1.9 Hz, 1H), 7.43 (d, J=15.8 Hz, 1H), 7.34 (d, J=2.5 Hz, 1H), 7.23 (dd, J=9.0, 2.5 Hz, 1H), 7.13-6.98 (m, 2H), 3.95 (s, 3H), 3.93 (s, 3H); MS-API: [M+H]⁺ 354 (calculated 353.08).

### (E)-7-Methoxy-3-(3-(2-methoxyphenyl)-3-oxoprop-1-enyl) quinolin-2(1H)-one (CTR-19)

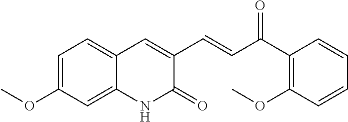 [SMILES: COc1ccc2cc(/C=C/C(=O)c3ccccc3OC)c(=O)[nH]c2c1]

The title compound was prepared by refluxing 3-(2-Chloro-7-methoxyquinolin-3-yl)-1-(2-methoxyphenyl)prop-2-en-1-one (QC-03) in aqueous acetic acid (70%) following the protocol described above under subsection II(c) in the general procedure for the synthesis of 3-(3-oxo-3-phenylprop-1-enyl)quinolin-2(1H)-ones.

Yield 83%; M.P. 227-229° C.; FT-IR (KBr) υ (cm⁻¹): 3144 (NH), 1656 (C═O), 1559 (C═C), 1167, 1021 (C—O—C); ¹H NMR (400 MHz, DMSO-d₆): δ 11.96 (s, 1H), 8.40 (s, 1H), 7.85 (d, J=16.0 Hz, 1H), 7.60-7.42 (m, 3H), 7.32-7.18 (m, 4H), 7.08 (t, J=7.4 Hz, 1H), 3.87 (s, 3H), 3.81 (s, 3H); MS-API: [M+H]⁺ 336.1 (calculated 335.12).

### (d) Synthesis of (E)-6-Methoxy-3-(3-(2-methoxyphenyl)-3-oxoprop-1-enyl) quinolin-2(1H)-one (CTR-20)

### 2-Chloro-6-methoxyquinoline-3-carbaldehyde (3d)

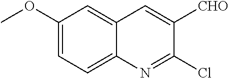

The title compound was prepared from N-(4-methoxyphenyl)acetamide (2d) following the protocol described above under subsection II(a) in the general procedure for the synthesis of 2 chloro-3-formyl quinolines.

Yield 63%; M. P. 145-146° C. (Lit. 146° C.) (Srivastava and Singh, 2005); FT-IR (ATR) υ (cm⁻¹): 3053 (Aromatic C—H), 2829 (aldehyde C—H), 1680 (C═O), 1574 (C═N), 1227, 1026 (C—O—C), 1051 (C—Cl); ¹H NMR (400 MHz, Chloroform-d): δ 10.53 (s, 1H), 8.63 (s, 1H), 7.95 (d, J=9.2 Hz, 1H), 7.50 (ddd, J=9.3, 2.9, 1.0 Hz, 1H), 7.18 (s, 1H), 3.94 (s, 3H); MS-API: [M+H]⁺ 222 (calculated 221.02).

### 3-(2-Chloro-6-methoxyquinolin-3-yl)-1-(2-methoxyphenyl)prop-2-en-1-one (QC-04)

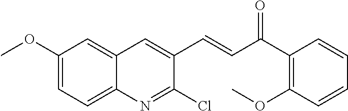 [SMILES: COc1ccc2nc(Cl)c(/C=C/C(=O)c3ccccc3OC)cc2c1]

The title compound was prepared by Claisen-Schmidt condensation 2-Chloro-6-methoxyquinoline-3-carbaldehyde (3d) with 2-methoxy acetophenone (4a) following the protocol described above under subsection II(b) in the general procedure for the synthesis of 2-chloroquinolinyl chalcones.

Yield 69%; M.P. 226-228° C.; FT-IR (ATR) υ (cm⁻¹): 3071 (Aromatic C—H), 2839 (Aliphatic C—H), 1666 (C═O), 1620 (C═C), 1234, 1020 (C—O—C), 1045 (C—Cl); ¹H NMR (400 MHz, Chloroform-d): δ 8.32 (s, 1H), 8.00 (d, J=15.9 Hz, 1H), 7.91 (d, J=9.3 Hz, 1H), 7.72-7.63 (m, 1H), 7.51 (ddd, J=8.9, 7.4, 1.9 Hz, 1H), 7.48-7.36 (m, 2H), 7.18-6.99 (m, 3H), 3.95 (s, 3H), 3.93 (s, 3H); MS-API: [M+H]⁺ 354.1 (calculated 353.08).

### (E)-6-Methoxy-3-(3-(2-methoxyphenyl)-3-oxoprop-1-enyl) quinolin-2(1H)-one (CTR-20)

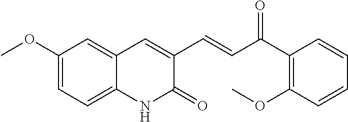 [SMILES: COc1ccc2[nH]c(=O)c(/C=C/C(=O)c3ccccc3OC)cc2c1]

The title compound was prepared by refluxing 3-(2-Chloro-6-methoxyquinolin-3-yl)-1-(2-methoxyphenyl)prop-2-en-1-one (QC-04) in aqueous acetic acid (70%) following the protocol described above under subsection II(c) in the general procedure for the synthesis of 3-(3-oxo-3-phenylprop-1-enyl)quinolin-2(1H)-ones.

Yield 83%; M.P. 227-229° C.; FT-IR (KBr) υ (cm⁻¹); 3155 (NH), 1652 (C═O), 1597, 1558 (C═C), 1164, 1022 (C—O—C); ¹H NMR (400 MHz, DMSO-d₆): δ 11.91 (s, 1H), 8.37 (s, 1H), 7.78 (d, J=15.9 Hz, 1H), 7.64 (d, J=8.8 Hz, 1H), 7.57-7.49 (m, 2H), 7.48-7.40 (m, 1H), 7.20 (d, J=8.5 Hz, 1H), 7.07 (t, J=7.7 Hz, 1H), 6.89-6.81 (m, 2H), 3.85 (s, 3H), 3.84 (s, 3H); MS-API: [M+H]⁺ 336.1 (calculated 335.12).

### (e) Synthesis of (E)-3-(3-(2,6-dimethoxyphenyl)-3-oxoprop-1-enyl)quinolin-2(1H)-one (CTR-25)

### 2-Chloroquinoline-3-carbaldehyde (3a)

The compound was prepared from acetanilide (2a) following the protocol described above under subsection II(a) in the general procedure for the synthesis of 2 chloro-3-formyl quinolines. Spectral data for the title compound is given above under subsection III(a).

### (E)-3-(2-chloroquinolin-3-yl)-1-(2,6-dimethoxyphenyl)prop-2-en-1-one (QC-09)

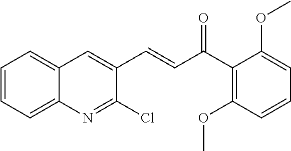 [SMILES: COc1cccc(OC)c1C(=O)/C=C/c1cc2ccccc2nc1Cl]

The title compound was prepared by Claisen-Schmidt condensation of 2-chloroquinoline-3-carbaldehyde (3a) with 2,6-dimethoxy acetophenone (4b) following the protocol described above under subsection II(b) in the general procedure for the synthesis of 2-chloroquinolinyl chalcones.

Yield 71%; M.P. 199-201° C.; FT-IR (ATR) υ (cm⁻¹): 3004 (Aromatic C—H), 2836 (Aliphatic C—H), 1650 (C═O), 1581 (C═C), 1223, 1020 (C—O—C), 1047 (C—Cl); ¹H NMR (400 MHz, Chloroform-d): δ 8.42 (s, 1H), 7.98 (d, J=8.5 Hz, 1H), 7.85 (d, J=8.3 Hz, 1H), 7.71-7.79 (m, 2H), 7.52-7.62 (m, 1H), 7.35 (t, J=8.4 Hz, 1H), 7.00 (d, J=16.3 Hz, 1H), 6.63 (d, J=8.5 Hz, 2H), 3.80 (s, 6H); MS-API: [M+H]⁺ 354.2 (calculated 353.08).

### (E)-3-(3-(2,6-dimethoxyphenyl)-3-oxoprop-1-enyl)quinolin-2(1H)-one (CTR-25)

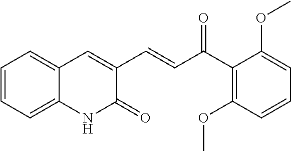 [SMILES: COc1cccc(OC)c1C(=O)/C=C/c1cc2ccccc2[nH]c1=O]

The title compound was prepared by refluxing 3-(2-chloroquinolin-3-yl)-1-(2,6-dimethoxyphenyl)prop-2-en-1-one (QC-09) in aqueous acetic acid (70%) following the protocol described above under subsection II(c) in the general procedure for the synthesis of 3-(3-oxo-3-phenylprop-1-enyl)quinolin-2(1H)-ones.

Yield 65%; M.P. 236-238° C.; FT-IR (ATR) υ (cm⁻¹): 3149 (NH), 1667 (C═O), 1591, 1558 (C═C), 1252, 1058 (C—O—C); ¹H NMR (400 MHz, DMSO-d₆): δ 12.00 (s, 1H), 8.41 (s, 1H), 7.66 (d, J=8.0 Hz, 1H), 7.47-7.55 (m, 1H), 7.32-7.41 (m, 2H), 7.21-7.31 (m, 2H), 7.18 (t, J=7.6 Hz, 1H), 6.73 (d, J=8.5 Hz, 2H), 3.69 (s, 6H); MS-API: [M+H]⁺ 336.2 (calculated 335.12).

### (f) Synthesis of (E)-3-(3-(2-ethoxyphenyl)-3-oxoprop-1-enyl)quinolin-2(1H)-one (CTR-32)

### 2-Chloroquinoline-3-carbaldehyde (3a)

The title compound was prepared from acetanilide (2a) following the protocol described above under subsection II(a) in the general procedure for the synthesis of 2 chloro-3-formyl quinolines. Spectral data for the compound is given above under subsection III(a).

### (E)-3-(2-chloroquinolin-3-yl)-1-(2-ethoxyphenyl)prop-2-en-1-one (QC-16)

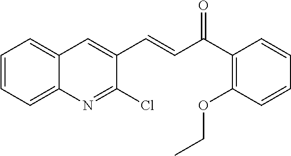 [SMILES: CCOc1ccccc1C(=O)/C=C/c1cc2ccccc2nc1Cl]

The title compound was prepared by Claisen-Schmidt condensation of 2-chloroquinoline-3-carbaldehyde (3a) with 2-ethoxy acetophenone (4i) following the protocol described above under subsection II(b) in the general procedure for the synthesis of 2-chloroquinolinyl chalcones.

Yield 73%; M.P. 128-130° C.; FT-IR (ATR) υ (cm⁻¹): 3055 (Aromatic C—H), 2931 (Aliphatic C—H), 1669 (C═O), 1596 (C═C), 1238, 1037 (C—O—C), 1042 (C—Cl); ¹H NMR (400 MHz, Chloroform-d): δ 8.42 (s, 1H), 7.98-8.07 (m, 2H), 7.84 (d, J=8.0 Hz, 1H), 7.75 (t, J=7.6 Hz, 1H), 7.70 (d, J=7.5 Hz, 1H), 7.55-7.61 (m, 2H), 7.48 (t, J=7.9 Hz, 1H), 7.05 (t, J=7.5 Hz, 1H), 6.99 (d, J=8.3 Hz, 1H), 4.16 (q, J=7.0 Hz, 2H), 1.44 (t, J=7.0 Hz, 3H); MS-API: [M+H]⁺ 338.2 (calculated 337.09).

### (E)-3-(3-(2-ethoxyphenyl)-3-oxoprop-1-enyl)quinolin-2(1H)-one (CTR-32)

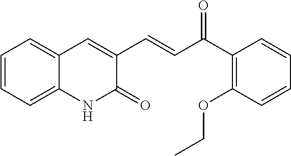 [SMILES: CCOc1ccccc1C(=O)/C=C/c1cc2ccccc2[nH]c1=O]

The title compound was prepared by refluxing 3-(2-chloroquinolin-3-yl)-1-(2-ethoxyphenyl)prop-2-en-1-one (QC-16) in aqueous acetic acid (70%) following the protocol described above under subsection II(c) in the general procedure for the synthesis of 3-(3-oxo-3-phenylprop-1-enyl)quinolin-2(1H)-ones.

Yield 77%; M.P. 199-201° C.; FT-IR (ATR) υ (cm⁻¹): 3128 (NH), 1651 (C═O), 1597, 1555 (C═C), 1169, 1023 (C—O—C); ¹H NMR (400 MHz, DMSO-d₆): δ 12.02 (s, 1H), 8.39 (s, 1H), 8.00 (d, J=15.8 Hz, 1H), 7.68 (dd, J=8.0, 1.3 Hz, 1H), 7.44-7.56 (m, 4H), 7.26-7.32 (m, 1H), 7.11-7.23 (m, 2H), 7.02 (td, J=7.5, 1.0 Hz, 1H), 4.12 (q, J=7.0 Hz, 2H), 1.31 (t, J=6.9 Hz, 3H). MS-API: [M+H]⁺ 320.2 (calculated 319.12).

### (g) Synthesis of (E)-3-(3-(2-ethoxyphenyl)-3-oxoprop-1-enyl)-6-methoxyquinolin-2(1H)-one (CTR-40)

### 2-Chloro-6-methoxyquinoline-3-carbaldehyde (3d)

The title compound was prepared from N-(4-methoxyphenyl)acetamide (2d) following the protocol described above under subsection II(a) in the general procedure for the synthesis of 2 chloro-3-formyl quinolines. Spectral data for the compound is given above under subsection III(d).

### (E)-3-(2-chloro-6-methoxyquinolin-3-yl)-1-(2-ethoxyphenyl)prop-2-en-1-one (QC-24)

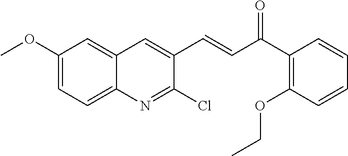 [SMILES: CCOc1ccccc1C(=O)/C=C/c1cc2cc(OC)ccc2nc1Cl]

The title compound was prepared by Claisen-Schmidt condensation of 2-chloro-6-methoxyquinoline-3-carbaldehyde (3d) with 2-ethoxy acetophenone (4i) following the protocol described above under subsection II(b) in the general procedure for the synthesis of 2-chloroquinolinyl chalcones.

Yield 74%; M.P. 151-153° C.; FT-IR (ATR) υ (cm⁻¹): 3058 (Aromatic C—H), 2930 (Aliphatic C—H), 1655 (C═O), 1622 (C═C), 1233, 1021 (C—O—C), 1048 (C—Cl); ¹H NMR (400 MHz, Chloroform-d): δ 8.31 (s, 1H), 7.99 (d, J=15.8 Hz, 1H), 7.90 (d, J=9.3 Hz, 1H), 7.68 (d, J=7.8 Hz, 1H), 7.53 (d, J=15.8 Hz, 1H), 7.47 (t, J=7.9 Hz, 1H), 7.39 (dd, J=9.3, 2.8 Hz, 1H), 7.01-7.09 (m, 2H), 6.98 (d, J=8.3 Hz, 1H), 4.15 (q, J=6.9 Hz, 2H), 3.93 (s, 3H), 1.42 (t, J=7.0 Hz, 3H); MS-API: [M+H]⁺ 368.2 (calculated 367.1).

### (E)-3-(3-(2-ethoxyphenyl)-3-oxoprop-1-enyl)-6-methoxyquinolin-2(1H)-one (CTR-40)

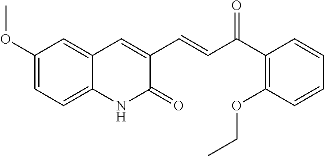 [SMILES: CCOc1ccccc1C(=O)/C=C/c1cc2cc(OC)ccc2[nH]c1=O]

The title compound was prepared by refluxing 3-(2-chloro-6-methoxyquinolin-3-yl)-1-(2-ethoxyphenyl)prop-2-en-1-one (QC-24) in aqueous acetic acid (70%) following the protocol described above under subsection II(c) in the general procedure for the synthesis of 3-(3-oxo-3-phenylprop-1-enyl)quinolin-2(1H)-ones.

Yield 86%; M.P. 233-235° C.; FT-IR (KBr) υ (cm⁻¹): 3166 (NH), 1652 (C═O), 1598, 1560 (C═C), 1245, 1023 (C—O—C); ¹H NMR (400 MHz, DMSO-d₆): δ 11.94 (s, 1H), 8.33 (s, 1H), 8.00 (d, J=15.8 Hz, 1H), 7.43-7.53 (m, 3H), 7.17-7.26 (m, 3H), 7.15 (d, J=8.3 Hz, 1H), 7.02 (t, J=7.5 Hz, 1H), 4.12 (q, J=6.8 Hz, 2H), 3.77 (s, 3H), 1.31 (t, J=7.0 Hz, 3H); MS-API: [M+H]⁺ 350.2 (calculated 349.13).

The foregoing syntheses are representative examples. Table 1 shows the chemical structures and names of 24 novel quinolone chalcone compounds which were synthesized and characterized in the present studies.

### Example 2: In Vitro and In Vivo Studies of the CTR Compounds

I. Materials and Methods

### Reagents

RPMI 1640, DME/F12, fetal bovine serum and antibiotic antimycotic solutions (Pen/Strep/Fungiezone) were purchased from Hyclone (Logan, Utah). The antibodies specific for the following proteins were purchased from Santa Cruz (Santa Cruz, Calif.): PARP (cleavage product), cdc2, phospho-cdc2 on Tyr15 or Thr161 residue, cyclin A, cyclin B, cyclin E, wee1, cdc25C, phospho-histone H3 (Ser10), α-tubulin, γ-tubulin and GAPDH. The antibodies specific for the following proteins were from Abcam (Cambridge, UK): phospho-cdc25C on Thr48 or Ser216, securin, BubR1, and cdc20. Alexafluor 488 (anti-mouse) and 568 (anti-goat) conjugated IgG and DRAQ5/DAPI were purchased from Molecular Probes/Invitrogen. Tubulin polymerization kits (BK004P) and purified porcine tubulin (T240) were purchased from Cytoskeleton Inc. (Denver, Colo.). All reagents used for the experiments were of analytical grade.

### Cell Lines and Cell Culture

All of the cell lines used were purchased from ATCC and cultured in RPMI-1640 supplemented with 10% fetal bovine serum and antibiotics (100 units penicillin/100 μg/ml streptomycin), unless stated otherwise. 1845B5 and MCF-10A non-cancer breast cell lines were cultured in DME/F12 medium supplemented with 10% fetal bovine serum, antibiotics and growth factors. KB-3-1 is a human epidermal carcinoma cell line and KB-C-2 is its isogenic multidrug-resistant cell line with over-expressing ABCB1/P-gp. The KB-C-2 cell line was originally established in the presence of increasing concentrations of colchicine. The human small cell lung carcinoma H69 cell line and its multidrug-resistant MRP1-overexpressing isogenic H69AR cell line were purchased from ATCC. The H69AR cell line was established in the presence of increasing concentrations of Adriamycin (doxorubicin). H69 cells grow as large multi-cell aggregates, making it difficult to accurately count cell numbers. Therefore, the cytotoxicity results obtained from H69AR cells were compared to SW1271, a small cell lung carcinoma cell line without a multidrug-resistant phenotype. The IL-6 dependent bortezomib-resistant ANBL6-BR cell line was further supplemented with 1 ng/ml of IL-6. The MCF10AT1 and MCF10CA1a cell lines are isogenic to the MCF10A cell line, and obtained from Dr. Valerie Weaver at the Center for Bioengineering and Tissue Regeneration, UCSF, CA, USA. MCF10AT1 is a premalignant cell line generated by transforming MCF10A with c-Ha-Ras; and MCF10CA1a was isolated by selecting malignant cells after MCF10AT1 cells were engrafted into mice (Liu & Lin 2004; Marella et al. 2009). The MCF10AT1 and MCF10CA1a cells were cultured in DMEM supplemented with 10% FBS (volume/volume). MDA-MB231TaxR cell line was generated in house by culturing MDA-MB231 cells in gradually increasing doses of paclitaxel over one-year period, and finally maintained at 100 nM paclitaxel. The drug-resistant cells were cultured in the absence of drug for at least one passage before carrying out experiments. All cells were maintained in a humidified incubator at 37° C. (5% CO₂/95% air). Cell line authentication was performed using short tandem repeat (STR) profiling.

### Sulforhodamine B (SRB)-Based Cytotoxicity Assay

For (anti)proliferation assays, 4,000-5,000 cells/well of the 96-well clustered dish were incubated for 16 hours, as described previously (Hu et al., 2008; Skehan et al., 1990). After 16 hours, culture medium was replaced with fresh medium containing different dilutions of test compounds dissolved in DMSO. Some wells were treated with 100 μl of 10% trichloroacetic acid (TCA) as a negative control and sham (medium with dimethyl sulfoxide; DMSO) treated cells were used as a positive control. After 72 hours post-incubation, medium was removed and cells were fixed with 10% TCA at 4° C. for 1 hour. TCA was removed and cells were washed with cold tap water, and plate was air-dried, followed by addition of 50 μl of 0.4% SRB staining solution to each well. After 30 minutes incubation, SRB staining solution was removed. Cells were washed with 1% acetic acid solution, and then washed with tap water to remove unbound staining solution, followed by air-drying. 200 μl of 10 mM (pH10.5) trizma base buffer was added to each well to solubilise macromolecules. SRB stained macromolecules were determined at a 540 nm wavelength using an automated plate reader (Synergy H4 Hybrid Multi-Mode Microplate Reader, BioTek, Winooski, Vt.). Cell growth (inhibition) was calculated by the following formula:

% cells proliferation=[(AT−CT)/(ST−CT)]×100

wherein AT=absorbance of treated cells, CT=absorbance of negative control cells, and ST=absorbance of sham treated cells. IC₅₀ values were calculated from sigmoidal dose-response curves generated by two independent biological replicates, with quadruplicate in each set by using Graph Pad Prism v.5.04 software. For combinational treatments against KB-C-2, MDA-MB231 or MB231TaxR cells, CTR compounds and paclitaxel or ABT-737 were used at different concentrations which were at or below the IC₅₀ values of single compounds. The combinational index (CI) was calculated as described previously (Chou 2006). If the CI values were less than, equal to or more than 1, it indicates a synergistic, additive or antagonistic effect respectively (Chou 2006). CI values were determined from four independent experiments.

### Cell Cycle Analysis by Flow Cytometry

Approximately 1×10⁶ cells per plate were seeded and grown overnight. Cells were then treated the next morning with test compounds, and harvested at the scheduled post-treatment times. The cell pellet was collected by centrifugation at 1,100 rpm (Allegra™ X-12 centrifuge, Beckman Coulter, Indianapolis, Ind.), followed by washing the cells twice with PBS, and fixing them with 75% ethanol for 12-24 hours at −20° C. Ethanol was removed by centrifugation at 11,000 rpm (Allegra™ X-12 centrifuge, Beckman Coulter); cells were suspended in PBS and centrifuged again at 11,000 rpm in the same rotor. The PBS was then removed and the cell pellet was resuspended and stained for 1 hour with propidium iodide (PI) staining solution (0.3% nonidet P-40, 100 μg/ml RNase A and 100 μg/ml PI in PBS). The DNA content in the different phases of the cell cycle was analysed by flow cytometry using Beckmann Coulter Cytomics FC500 (Mississauga, ON, Canada). The reversibility of drug effects was determined as follow: HeLa cells treated with a CTR compound for 12 hours were washed twice with 1×PBS, and then released them into pre-warmed drug-free complete medium for scheduled durations. The cells were then examined by confocal microscopy for their morphology or subjected to cell cycle analysis by flow cytometry after cells (DNA) were stained with propidium iodide.

### Cell Synchronization

Synchronization at the G1/S border was achieved by double thymidine block (DT). Briefly, exponentially growing cells were treated with 2.0 mM thymidine for 18 hours, followed by incubation for 11 hours in drug-free complete medium, by which most cells are at mid-late G1 phase. The cells were then incubated for another 14 hours in 2.0 mM thymidine to arrest them at the G1/S border. To arrest cells at the prometa phase, cells were maintained for 18 hours in the complete medium containing nocodazole (50 ng/ml).

### Immunofluorescent Staining

Cells on coverslips placed on the bottom of 35 mm tissue culture plates or 6-well clustered dishes were treated for 12-24 hours with the CTR compounds to be tested. Subsequently, the cells were fixed with 100% methanol for 15 minutes and washed with 1×PBS three times. Cells were then “blocked” with 3% BSA or 1% (v/v) FBS plus 1×PBST (1×PBS buffer containing 0.1% (v/v) Triton X-100 or 0.2% Tween 20), and incubated overnight at 4° C. with primary antibodies, with gentle agitation. Unbound primary antibodies were washed off with PBST, and a secondary antibody was added for 1 hour in the dark. Secondary antibodies were conjugated to Alexa 488 or 568. DNA was counterstained with DRAQ5 or DAPI. Subsequently, coverslips were washed three times with 1×PBST for 10 minutes each, followed by mounting them onto slides with 90% glycerol in 1×PBS. Each slide was visualized with a Carl Zeiss 510 Meta laser scanning microscope or an Axioscope. Image analysis was done with an LSM image examiner equipped with the microscopes (Carl Zeiss, Toronto, ON, Canada).

### Western Blotting

Exponentially growing cells were collected by centrifugation at 1,100 rpm (Allegra™ X-12 centrifuge, Beckman Coulter) at scheduled time points post-treatment. Cells were washed three times with PBS by centrifugation under the same conditions, followed by cell lysis for 10-15 minutes on ice in 100 μl Lysis buffer (150 mM NaCl, 5 mM EDTA, 1% triton X-100, 10 mM tris pH 7.4, 1 mM PMSF, 5 mM EDTA and 5 mM protease inhibitor). Cell extracts were centrifuged at 11,000 rpm (Allegra™ X-12 centrifuge, Beckman Coulter) for 10 minutes at 4° C. Supernatant was collected and the protein concentration was measured using a BCA assay kit according to the supplier's specifications (Thermo Fisher Scientific, Waltham, Mass.). Cell lysates were then diluted with 2× Iaemmli sample buffer and boiled for 5 minutes at 95-100° C. 30-40 μg protein was loaded on 8% or 10% polyacrylamide gel and resolved by electrophoresis. Proteins were then electronically transferred to a PVDF membrane for 75 minutes at 24 volts, followed by “blocking” with 5% skim milk for 1 hour. Proteins were incubated with primary antibody overnight at 4° C. in 0.1% TBST buffer containing 5% skim milk. The membrane was washed three times with 0.1% TBST buffer and incubated for 1 hour with secondary antibody in TBST buffer containing 5% skim milk. The membrane was then washed with TBST buffer three times, and the signals were visualised on X-ray film using an ECL chemiluminescence kit (Super Signal West pico, Thermo Fisher Scientific).

### Immunoprecipitation

Cell lysates were prepared in 1×IP buffer (20 mM Tris-HCl, pH 7.5, 150 mM NaCl, 1 mM EDTA and 1% (v/v) Triton X-100, supplemented with 10 mM sodium fluoride, 1 mM sodium orthovanadate and protease inhibitors) and pre-cleared for 3 hours at 4° C. by gentle agitation. Subsequently, immunoprecipitation was performed with an antibody overnight at 4° C., followed by mixing with protein A/G agarose beads for an additional 5 hours. The complex was washed five times with Lysis buffer, boiled for 5 minutes, and resolved by SDS-PAGE, followed by immunostaining with an appropriate antibody.

### Microtubule Polymerization Assay

The effects of the tested CTR compounds of the present disclosure on the assembly of purified tubulin were determined using a tubulin polymerization kit according to the manufacturer's instructions (Cytoskeleton Inc., Denver, Colo.). Paclitaxel (provided in the same kit), nocodazole, and colchicine (Santa Cruz, Calif.) were used as controls for the assay. The absorbent-based assay kit is based on the principle that the light scattered by the microtubules is directly proportional to the polymer mass of the microtubules when measured at 37° C. at a wavelength of 340 nm. The fluorescence-based assay kit is on the principle that fluorescent reporter molecules are incorporated into microtubules as the polymerization process being occurred. The fluorescence enhancement was measured for one hour at one-minute intervals, at the excitation of 350 nm and the emission of 430 nm. Absorbance or fluorescence was measured with an automated plate reader (Synergy H4 Hybrid Multi-Mode Microplate Reader, Bio-Tek).

### Differential Tubulin Extraction

A two-step extraction procedure was used to separately isolate soluble and polymerised tubulin fractions from sham treated or treated with compounds, as described previously (Tokesi et al., 2010). Briefly, exponentially growing cells were treated with 50 nM of paclitaxel, 50 ng/ml nocodazole, 3.0 μM of CTR-17, or 1 μM of CTR-20 for 12 hours. Cells were then harvested and lysed with pre-warmed microtubule stabilizing buffer (80 mM PIPES, pH 6.8, 1 mM MgCl₂, 1 mM EGTA, 0.5% Triton X-100, 10% glycerol, and protease inhibitor cocktail). After a brief centrifugation at 2,500 rpm for 5 minutes at room temperature (Allegra™ X-12 centrifuge, Beckman Coulter), the tubulin heterodimers in the soluble fractions were separated from supernatant by centrifugation as above. To ensure the soluble tubulin was completely extracted, the cell pellet was washed once again with the microtubule stabilizing buffer; the supernatant fractions were pooled; and finally polymerized tubulin complexes were extracted using microtubule destabilizing buffer (20 mM Tris, pH 7.4, 150 mM NaCl, 1% Triton X-100, 10 mM CaCl₂, and protease inhibitor cocktail). The extract was cleared by centrifugation to obtain an insoluble microtubule fraction (2,500 rpm for 5 minutes at room temperature with Allegra™ X-12 centrifuge, Beckman Coulter). An equal amount of protein for each sample was resolved by SDS-PAGE, followed by Western blot and densitometry-based analyses using AlphaEaseFC 4.0 software.

### Determining the Dissociation Constant of CTR-Tubulin Binding

Purified tubulin (0.4 μM) was dissolved in 25 mM PIPES buffer (pH 6.8) and incubated in the presence or absence of different concentrations of CTR-17 (or CTR-20) for 30 minutes at 37° C. The intrinsic fluorescence of the tryptophan residues in the tubulin heterodimers was monitored by excitation of the reaction mixture at 295 nm and the emission spectra at the 315-370 nm wavelength range. All measurements were corrected for the inner filter using the formula F_{corrected}=F_{observed}×antilog [(Aₑₓ+Aₑₘ)/2], where Aₑₓ and Aₑₘ are the absorbance of the reaction mixture at the excitation and emission wavelengths, respectively. Graph Pad Prism software was used to determine the dissociation constant of the CTR compounds binding to tubulin using the following formula

$$Δ F = \frac{Δ Fm a x \times C}{Kd + C}$$

wherein ΔF is the changes in fluorescence intensity of tubulin when bound with the CTR compounds, ΔFₘₐₓ is the maximum change in the fluorescence intensity when tubulin is bound with the compounds, C is the concentration of the CTR compounds, and K_{d} is the dissociation constant of the CTR compounds bound to tubulin.

### Competitive Binding Assays

For the BODIPY FL vinblastine competition assay, 25 μM each of CTR-17, CTR-20, colchicine, and vinblastine was incubated with purified tubulin for 1 hour at 37° C. Subsequently, BODIPY FL vinblastine was added to the tubulin complex to a final concentration of 5.0 μM and the mixture incubated for 30 minutes at 37° C. For the colchicine competition assay, tubulin was incubated with different concentrations of each compound for 1 hour at 37° C. Subsequently, colchicine was added to the CTR-tubulin or vinblastine-tubulin equilibria to a final concentration of 1.0-8.0 μM. Fluorescence was monitored using an automated plate reader (Synergy H4 Hybrid Multi-Mode Micro plate Reader, Bio-Tek). For the vinblastine competition assay, fluorescence was monitored by excitation of the reaction mixture at 490 nm and the emission spectra at the 510-550 nm range. For the colchicine competition assay, the fluorescence of the tubulin complexes was determined with an excitation wavelength of 360 nm and emission wavelength at 430 nm. A modified Dixon plot was used to analyze the competitive inhibition of colchicine binding to tubulin and to determine the inhibitory concentration (Ki) of the CTR compounds.

### Molecular Modeling

The Molecular Operating Environment (MOE) (Chemical Computing Group Inc, Montreal, Quebec, Canada) was used to predict the interaction mode of CTR compounds to the colchicine binding domain of the β-tubulin subunit. The crystal structure of the tubulin-colchicine complex (PDB Code: 1 SA0) was used as the target structure and was subjected to energy minimization and protonation using the same software. The protocol for docking was adopted from the MOE website, induced fit protocol was used. The best docking pose was determined based on the free energy for binding. The contributions of H-bonds, hydrophobic, ionic and Van der Waals interactions were taken into consideration when calculating free energy values.

### III. Animal Work

### Mice, Cells and Reagent/Protocols

Five-week old female CD-1 and ATH490 (strain code 490) athymic nude mice were purchased from Charles River (Quebec, Canada). The MDA-MB-231 human metastatic breast cancer cells were obtained from the American Tissue Culture Collection (ATCC, Manassas, Va., USA). Cells were maintained under humidified conditions at 37° C. and 5% CO₂ in DMEM high glucose medium (ATCC) supplemented with 10% fetal bovine serum and antibiotics.

For paclitaxel treatments, 40 mg/ml stock solution of paclitaxel (Sigma, MO) was prepared in DMSO. Just before administration to mice, the paclitaxel stock solution was diluted ten-fold in a buffer containing 10% DMSO, 12.5% Cremophor, 12.5% ethanol, and 65% saline-based diluent (0.9% sodium chloride, 5% polyethylene glycol, and 0.5% tween-80) which is defined as vehicle (Huang et al., 2006). Alanine transaminase (ALT, SUP6001-c)/Aspartate transaminase (AST, SUP6002-c) color endpoint assay kits were purchased from ID Labs Biotechnology (London, Ontario, Canada). Elevation of ALT and AST levels in serum samples was used as an indicator of liver damage/injury.

### Anti-Tumor Activity of CTR Compounds in Xenograft Mice

To determine the anti-tumor activity of CTR compounds in animals, a xenograft model of human breast cancer cells in athymic nude mice was established. Exponentially growing MDA-MB-231 metastatic breast cancer cells were harvested and counted for inoculation into mice. Each mouse was subcutaneously injected at the flank with 10×10⁶ cells in 0.2 ml ice cold 1×PBS. When tumor size reached 4-5 mm in diameter (n=4-5 per group), mice were randomly assigned into several groups as described herein.

Animals were monitored for food and water consumption every day, and their body weights and tumor volumes were measured twice per week. Tumor volumes were measured with a digital caliper and were determined by using the following formula: ½ length×width². Blood samples were collected via cardiac puncture and processed further for ALT and AST measurements. The animals were then immediately euthanized by carbon dioxide. Tumors and vital organs (spleen, kidney, liver and lung) were collected and fixed in 10% buffered formalin at 4° C. overnight before being processed for paraffin embedding. The paraffin-embedded blocks were then cut into 4-5 μm thick sections. Each section of tumors and organs was stained with hematoxylin and eosin (H&E).

### Toxicity Study in Animals

Changes in body weight, hemoglobin (Hb) and the amount and ratio of alanine transaminase (ALT)/aspartate transaminase (AST) were used to measure toxic effects. In addition, vital organs (liver, spleen, kidney and lung) were analyzed by fluorescent microscopy after they were harvested, fixed, processed, paraffin-embedded, sectioned, and stained as described above.

### Statistical Analyses

All values are mean±S.E.M of at least three independent experiments. Analyses were performed using GraphPad Prism software (GraphPad Software, Inc). Comparison between the groups was made by p value determination using one-way ANOVA. A p value of <0.05 was considered to be statistically significant.

### IV. Results

Table 2 contains a summary of the results from the initial screening of four CTR compounds using breast cancer cells (MDA-MB-231, MDA-MB-468, MCF-7) and non-cancer breast cells (184B5) determined by SRB assays. As can be seen from the results in Table 2, CTR-17, -18, -19, and -20 are much more effective than chloroquine or cisplatin, the two reference compounds used in this experiment, CTR-17 and CTR-20 were observed to preferentially kill cancer cells over non-cancer cells up to 26 times (MDA-MB-468/K562 versus MCF-10A) and 24 times (HeLa versus MCF-10A), respectively. In contrast, cisplatin kills cancer and non-cancer cells with similar efficacy.

Table 3 contains a summary of the results on the antiproliferation effects of CTR-17 and CTR-20 on other cancer cell lines. All cell lines were authenticated on Apr. 10 & Jul. 13, 2015 by STR profiling of gDNA. As can be seen from the results in Table 3, CTR-17 and CTR-20 effectively kill many different cancer cells including brain cancer (U87MG), temozolomide-resistant glioblastoma (T98G), lung cancer (NCI-H1975, A549), multiple myeloma (RPMI-8229), urinary bladder cancer (UC3), and kidney cancer cell lines (HEK293T). The IC₅₀ values of CTR-20 on the RPMI-8226-BR (bortezomib-resistant) and ANBL6-BR (bortezomib-resistant multiple myeloma) cell lines were also determined in a separate experiment and found to be 0.28±0.03 μM and 0.76±0.28 μM, respectively.

Table 4 contains summary of the results on the anti-proliferation effects of 16 novel CTR compounds (CTR-21 to CTR-40). These CTR compounds killed MDA-MB231, MCF-7, HeLa and RPMI-8226 cancer cell lines, IC₅₀ values ranging from 5.34 nM (CTR-21 against RPMI-8226) to 2.69 μM (CTR-27 against MDA-MB231). For example, the IC₅₀ of CTR-21 in HeLa and RPMI-8226 cells was 11.93±1.40 and 5.34±0.89 nM, respectively. Similarly, CTR-32 was also effective as its IC₅₀ values were 12.88±0.35 and 6.29±1.43 nM, respectively, against HeLa and RPMI-8226 cells.

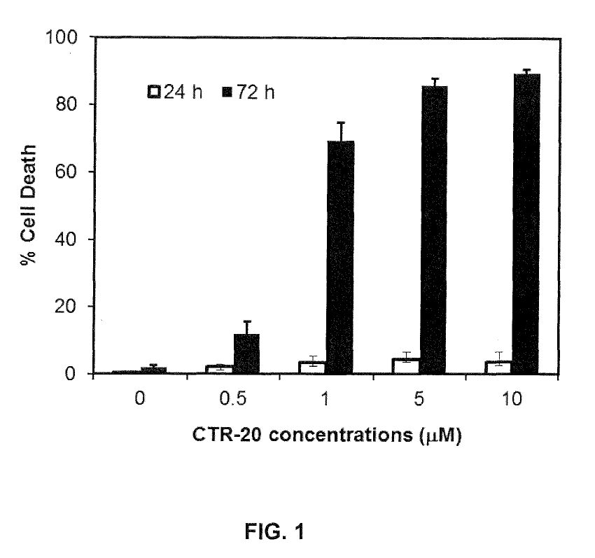

As can be seen from the results in FIG. 1, CTR-20 induced cell death in a time- and dose-dependent manner when asynchronously growing HeLa S3 cells were treated with CTR-20 at different concentrations (0, 0.5, 1.0, 5.0 and 10.0 μM) for 24 or 72 hours (h). Cell survival/death was determined by trypan blue exclusion assays. The treatment of HeLa cells with 1 μM of CTR-20 resulted in ˜70% death by 72 hours post-treatment.

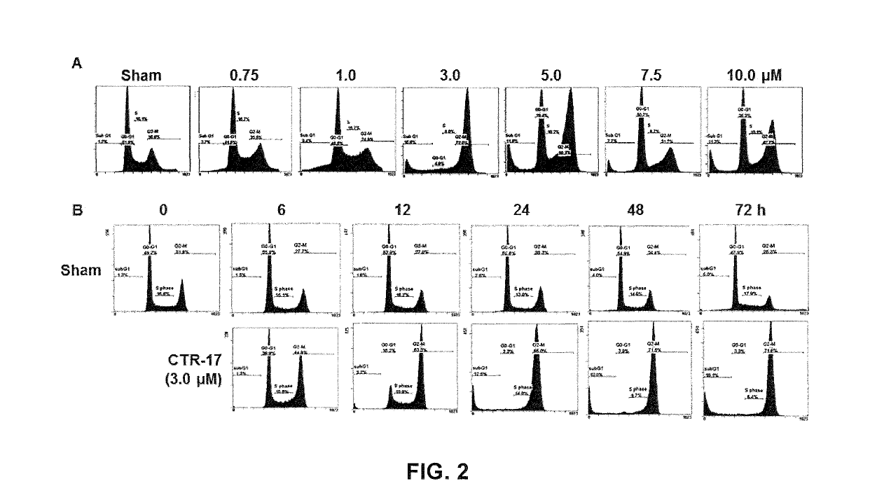

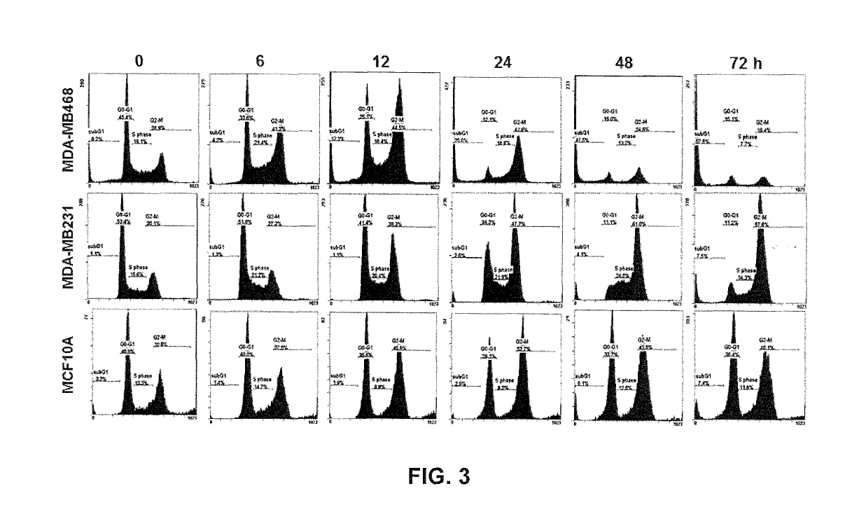

As can be seen from the results in FIG. 2, CTR-17 arrested cell cycle around the G2/M phase. FIG. 2A shows flow cytometry profiles of HeLa cells at 72 hours post-treatment with different concentrations (μM) of CTR-17. FIG. 2B shows cell cycle profiles at different time points after asynchronous HeLa cells were treated with 3.0 μM CTR-17. The majority of HeLa cells arrested around the G2/M phase by 12 hours post-treatment with 3 μM CTR-17.

While not wishing to be limited by theory, the differential effects of CTR-17 on cancer and non-cancer cells may be in part due to their differences in cell cycle arrest in response to this compound. Two breast cancer cell lines (MDA-MB-468 and MDA-MB-231) and one non-cancer breast cell line (MCF-10A) were treated with 3.0 μM CTR-17 for 0-72 hours, stained with propidium iodide, and their cell cycle profiles analyzed by flow cytometry. The results are shown in FIG. 3. The MDA-MB-468 metastatic breast cancer cells started to accumulate around G2/M by 6 hours post-treatment with 3 μM CTR-17, followed by massive cell death by 48 hours post-treatment. In MDA-MB-231, the G2/M population was accumulated much slower under the same conditions. Nevertheless, most of the MDA-MB-231 cells were arrested around G2/M by 48 hours post-treatment. Although the G2/M population was enriched, the non-cancer MCF-10A cells were never completely arrested in any cell cycle compartment.

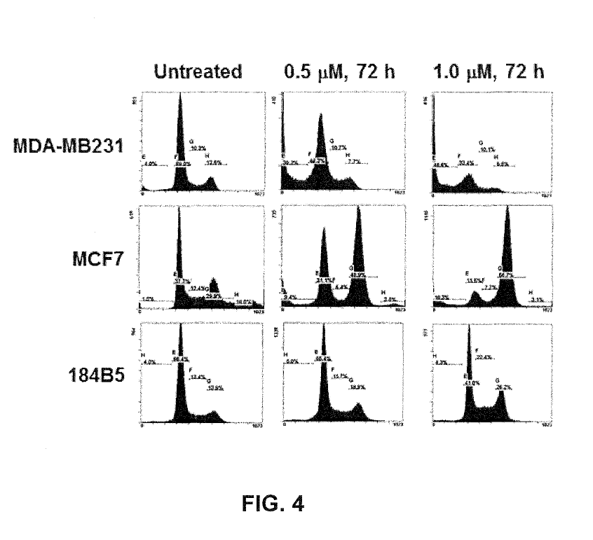

As can be seen from the results in FIG. 4, CTR-20 selectively caused cell cycle arrest and cell death in cancer, but not in non-cancer cells. Asynchronous breast cancer cells (MDA-MB-231 and MCF-7) and their matching non-cancer breast cells (184B5) were treated with CTR-20 at 0.5 or 1 μM for 72 hours. Cells were then collected, fixed and stained with propidium iodide for cell cycle analysis by flow cytometry. Most of the MDA-MB-231 cells were dead within 72 hours in the presence of 1 μM CTR-20. While not wishing to be limited by theory, the profile of the sub-G1 DNA content suggests that the cell death may be by apoptosis. Most of the MCF-7 breast cancer cells were also arrested around G2/M, although they were not yet dead by 72-hours post-treatment. In contrast to the two cancer cell lines, 184B5 non-cancer breast cells were not significantly affected by 1 μM CTR-20 under the same experimental conditions.

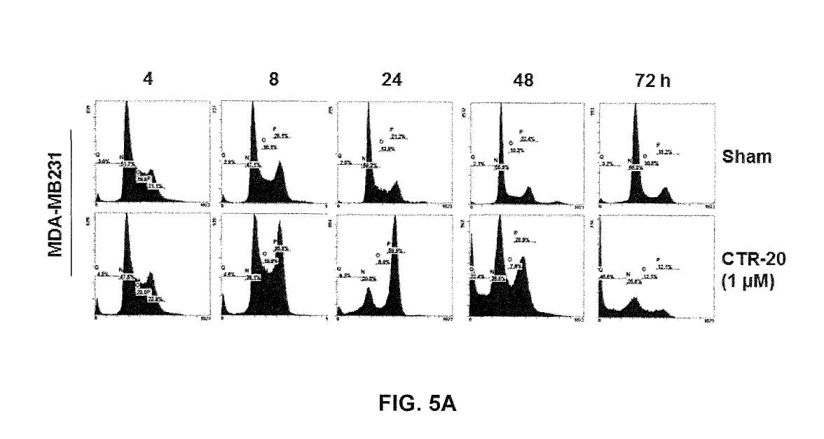

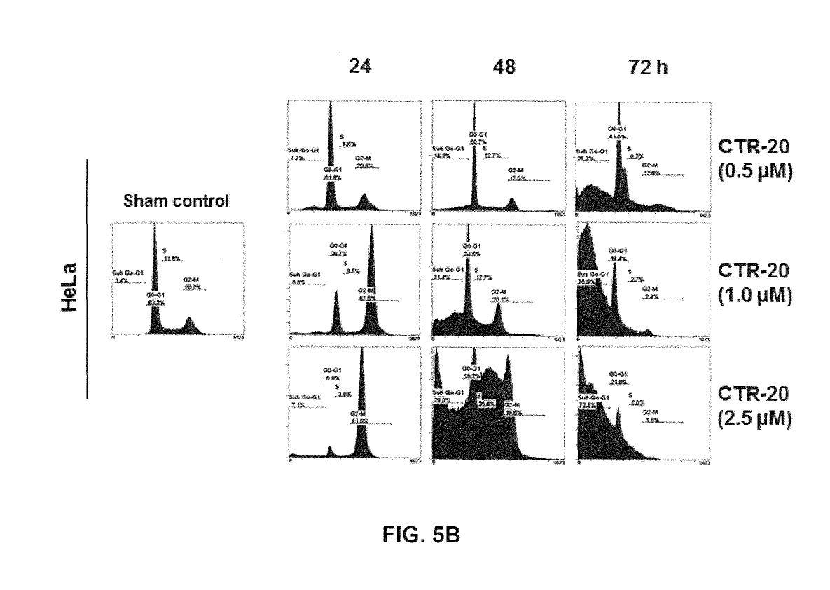

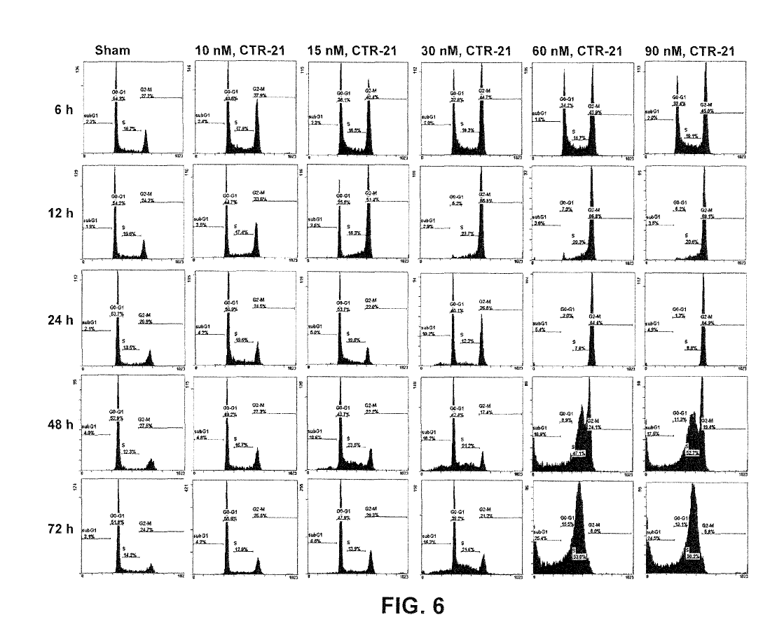

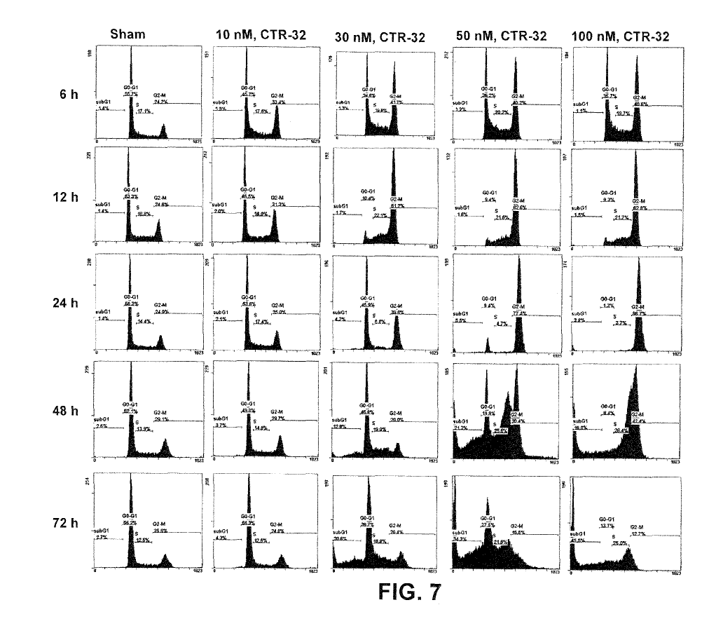

As can be seen from the results in FIG. 5, the treatment of cancer cells with 1 μM CTR-20 resulted in cell cycle arrest around G2/M phase by 24 hours and massive cell death by 72 hours post-treatment. Cells were treated with 1 μM CTR-20 for MDA-MB-231 (FIG. 5A) and 0.5, 1.0 or 2.5 μM for HeLa S3 (FIG. 5B) for 4, 8, 24, 48 and 72 hours (MDA-MB-231) or 24, 48 and 72 hours (HeLa S3). At each time point, cells were collected, fixed with formaldehyde, and then stained with propidium iodide, followed by flow cytometry. As can be seen in FIG. 5, CTR-20 at 1 μM arrested both MDA-MB-231 and HeLa cells around G2/M by 24-hour post-treatment. Most of these cells died with sub-G1 DNA content by 72-hour post-treatment. K562 leukemic cells also showed a similar pattern of cell cycle arrest and cell death as we found it in a separate experiment.

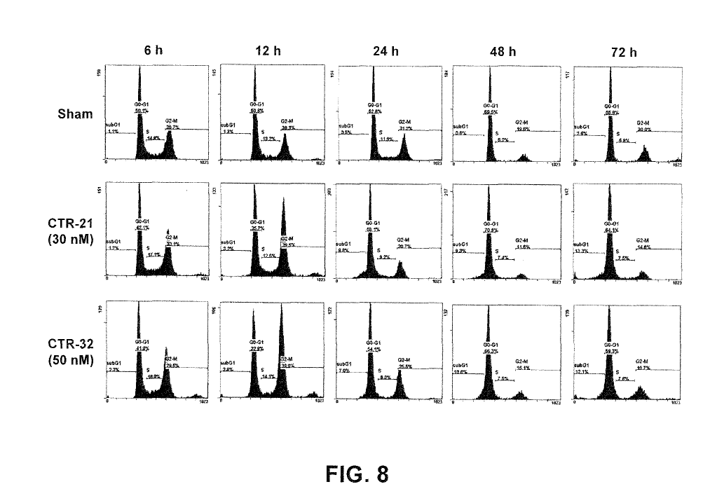

As shown in FIGS. 6 and 7, CTR-21 and CTR-32, similarly to CTR-17 and CTR-20, arrested cell cycle at G2/M. HeLa cells treated with 30 nM of CTR-21 or CTR-32 transiently arrested at G2/M. However, cells never returned to G1 in a normal fashion when the concentrations of CTR-21 and CTR-32 were increase to 60 nM and 50 nM, respectively. In addition, the flow cytometry profiles at 48 and 72 hours post-treatment indicated that 60 nM CTR-21 or 50 nM CTR-32 caused uneven cell division and cell death.

FIG. 8 shows that treatment of the MCF10A non-cancer cells with CTR-21 (30 nM) or CTR-32 (50 nM) had no noticeable ill effects, except a transient arrest in G2/M at 12-hour post-treatment.

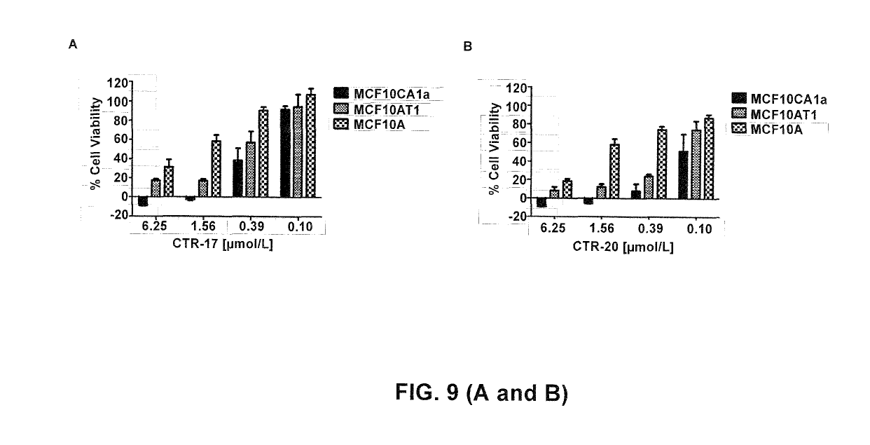

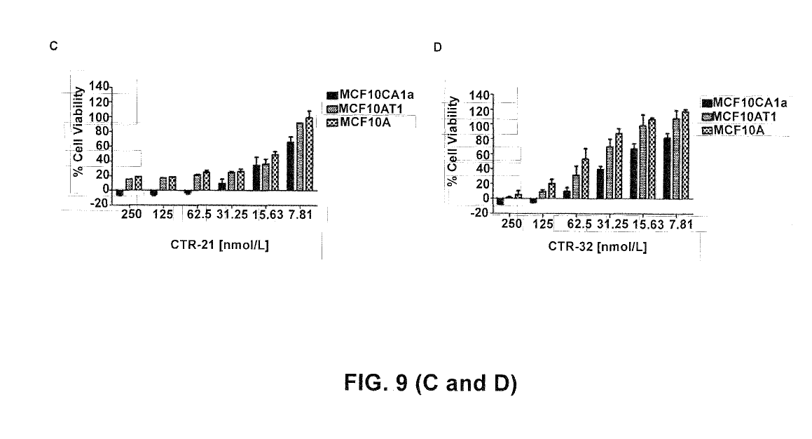

All four CTR compounds examined, (A) CTR-17, (B) CTR-20, (C) CTR-21 and (D) CTR-32, preferentially killed the fully malignant MCF10CA1a breast cancer cells over the premalignant MCF10AT1 and the non-cancer MCF10A breast cells. For example, the cell survival rates at 0.39 μM of CTR-17/CTR-20 were 39%/10%, 60%/20%, and 95%/75%, for MCF10Ala, MCF10AT1 and MCF10A, respectively (FIGS. 9A & B). Similarly, the cell viability at 31.25 nM of CTR-21/CTR-32 was 10%/40%, 24%/70%, and 26%/87% for MCF10Ala, MCF10AT1 and MCF10A, respectively (FIGS. 9C & D).

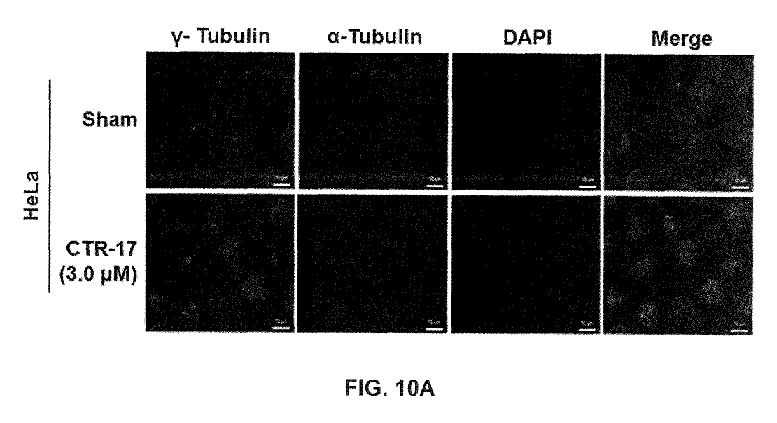

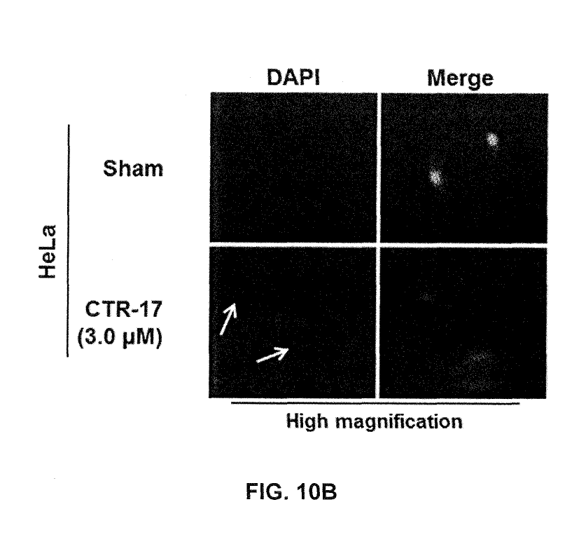

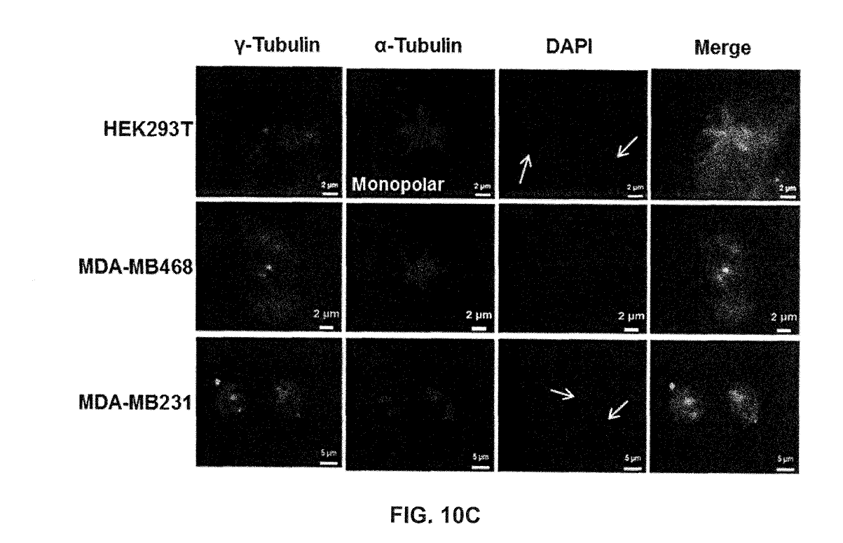

CTR-17 caused monopolar centrosomes, defects in chromosome alignment, and uneven chromosomal segregation (FIG. 10). Cells were treated with 3.0 μM CTR-17 for 12 hours, fixed in methanol, and stained with an antibody specific for γ-tubulin (green in a color image) or α-tubulin (red in a color image), and then the DNA counterstained with DAPI (blue in a color image). FIGS. 10A and 10B (higher magnification) show exemplary images of HeLa cells that were sham-treated or treated with 3 μM CTR-17. White arrows on FIG. 10B denote uneven alignment/segregation. FIG. 10C shows exemplary images of HEK293T, MDA-MB-468 and MDA-MB-231 cells that were treated with 3 μM CTR-17 in the same manner as in FIGS. 10A and 10B. White arrows on FIG. 10C denote the failure of proper alignment or uneven segregation of chromosomes. As can be seen from the results shown in FIG. 10, most cells treated with 3.0 μM of CTR-17 showed monopolar centrosomes, the failure of proper alignment at the centre plate, and uneven chromosome segregation. These abnormal phenomena were observed in all of the cancer cell lines examined thus far, including HeLa, HEK293T, MDA-MB-468 and MDA-MB-231 cell lines.

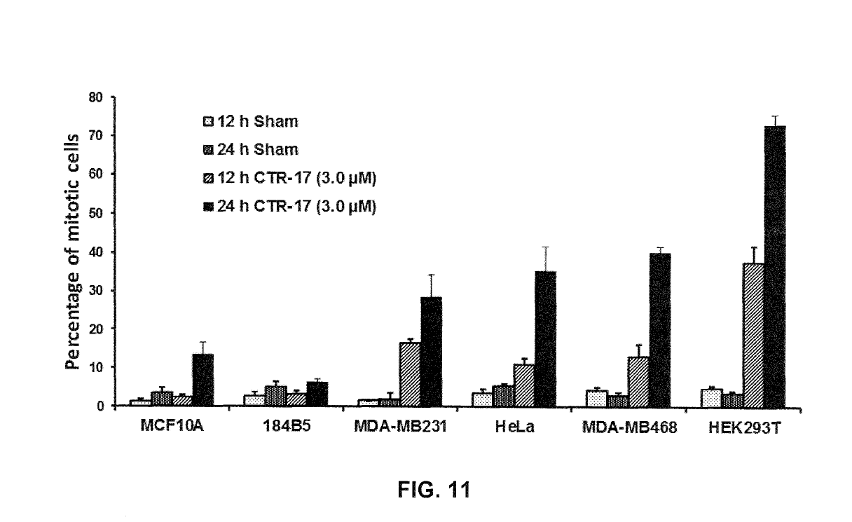

The cancer-specific increase of mitotic cells in response to CTR-17 was correlated with the accumulation of cells with monopolar centrosomes and abnormal chromosome alignment/segregation (FIG. 11, Table 5). Cells were either sham-treated or treated with 3.0 μM CTR-17 for 12 hours or 24 hours, followed by analyses of cell cycle progression, centrosome abnormalities, and chromosome alignment/segregation. FIG. 11 is a plot showing that the treatment of cancer cells with CTR-17 resulted in the accumulation of mitotic cells in a time-dependent manner. The mitotic index was determined by fluorescence microscopic analysis of at least 200 cells for each cell type, and data was expressed as percentage mean±S.E.M. of at least two independent experiments. Actual numbers taken from the plot in FIG. 11 are shown in Table 5. Table 5 does not include sham controls and non-cancer cells (MCF-10A and 184B5) since total numbers of mitotic cells in these groups were too small to make meaningful statistical comparison as these cells do not arrest at mitosis. As can be seen from this data, in response to 3 μM of CTR-17, cancer cells, but not non-cancer cells (MCF-10A, 184B5), were accumulated at mitotic phase with monopolar centrosomes or abnormal chromosome alignment/segregation.

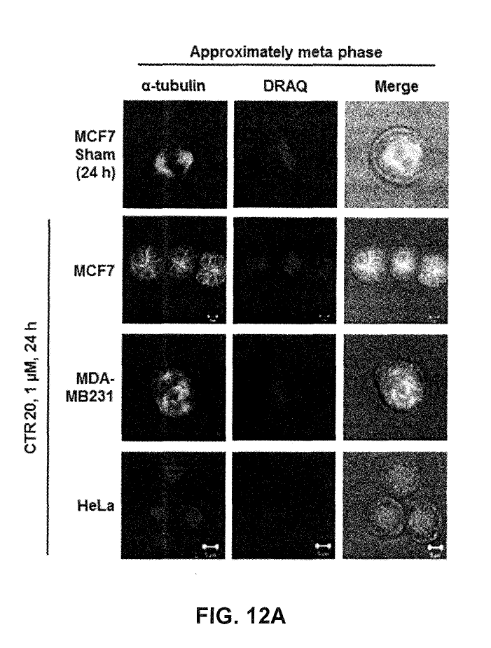

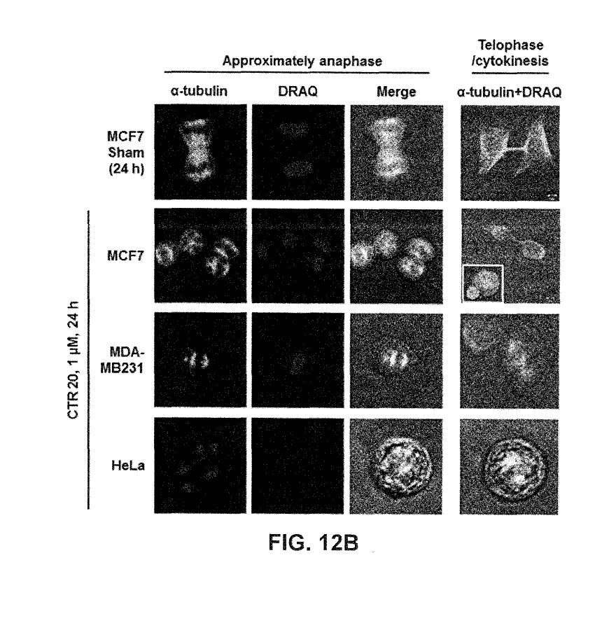

Cells treated with CTR-20 showed monopolar centrosomes or abnormal chromosome alignment and segregation (FIG. 12). Asynchronously growing MCF-7, MDA-MB-231 and HeLa S3 cells were treated with 1 μM CTR-20 for 24 hours. Cells were then collected, fixed with methanol, and incubated with an antibody specific for α-tubulin (green or red in color images), which was then counterstained DNA with DRAQ5 (red or blue in color images). The internal box in one of the MCF-7 samples in FIG. 12B shows uneven cell division. As can be seen from the exemplary images in FIG. 12, cancer cells treated with 1 μM CTR-20 also showed multipolar centrosomes and uneven cell division.

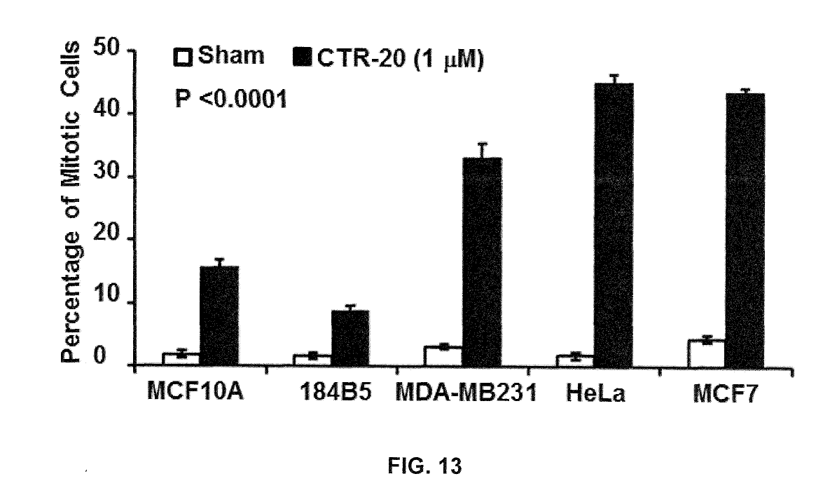

The number of cells containing monopolar centrosomes and chromosomes with defective alignment and uneven segregation dramatically increased in response to CTR-20 (FIG. 13, Table 6). As shown in FIG. 13, CTR-20 caused cell cycle arrest at mitosis in a cancer cell-specific manner. Asynchronously growing cells were treated with 1 μM CTR-20 for 24 hours, followed by examining cells arrested in mitosis. The percentage of mitotic cells was calculated based on the examination of at least 250-400 cells. Cells treated with 1 μM CTR-20 for 24 hours on coverslips were fixed with ice-cold methanol, immunostained with an α-tubulin antibody, and then counterstained DNA with DRAQ5 prior to observing by florescent microscopy (Table 6). Table 6 does not include sham controls and non-cancer cells (MCF-10A and 184B5) since the total numbers of mitotic cells in these groups were too small to make meaningful statistical comparison as they do not arrest at mitosis. As can be seen from FIG. 13, CTR-20 at 1 μM caused the accumulation of mitotic cells in cancer but not in non-cancer cells (MCF-10A, 184B5). Similarly, cells treated with 1 μM CTR-20 accumulated monopolar centrosomes and defective chromosomal alignment and segregation in a cancer-specific manner.

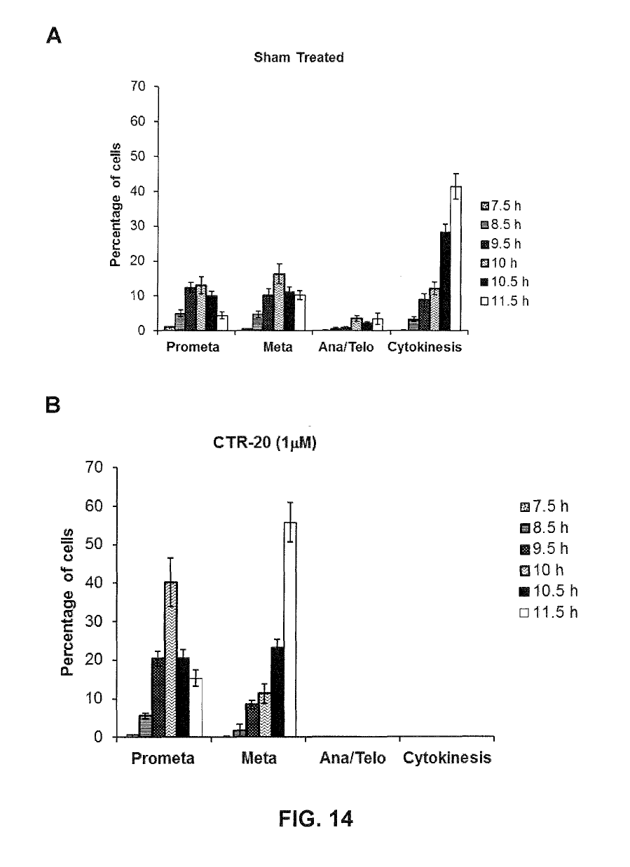

CTR-20 did not delay cells' entry into prometaphase/metaphase where they were eventually arrested (FIG. 14). HeLa S3 cells growing on cover slips were synchronized by double thymidine treatment (see materials and methods). Cells were then released into fresh medium either in the absence (Sham; FIG. 14A) or presence of 1.0 μM CTR-20 (FIG. 14B) for the duration of 7.5, 8.5, 9.5, 10, 10.5 or 11.5 hours. At the scheduled time points, cells were fixed with ice-cold methanol and immunostained with an antibody specific for α-tubulin, followed by counterstaining DNA with DRAQ5. Finally, cells were observed under a fluorescent microscope (Axio) at 40× objective. At least 15 fields were analyzed for each sample. As can be seen from FIG. 14B, cells treated with 1 μM CTR-20 normally progressed through the cell cycle until they reached prometaphase; however, they were accumulated in prometaphase by 9.5-10 hours and in metaphase by 11.5 hours post-G1/S in the presence of the drug. Unlike the sham control (FIG. 14A), very little cells were at the anaphase-cytokinesis cell cycle compartment by even 11.5 hours post-G/S in the presence of 1 μM CTR-20 (FIG. 14B), indicating that the metaphase arrest caused by CTR-20 is very effective. In contrast, sham-treated cells (FIG. 14A) started to progress into anaphase/telophase and cytokinesis as early as 7.5 hours post-release from G1/S arrest. Most cells in sham control (FIG. 14A) were in cytokinesis or interphase by 11.5 hours. This data is consistent with the notion that cells arrested in prometaphase in the presence of 1 μM CTR-20 eventually progressed to mitotic phase where they are “permanently” arrested.

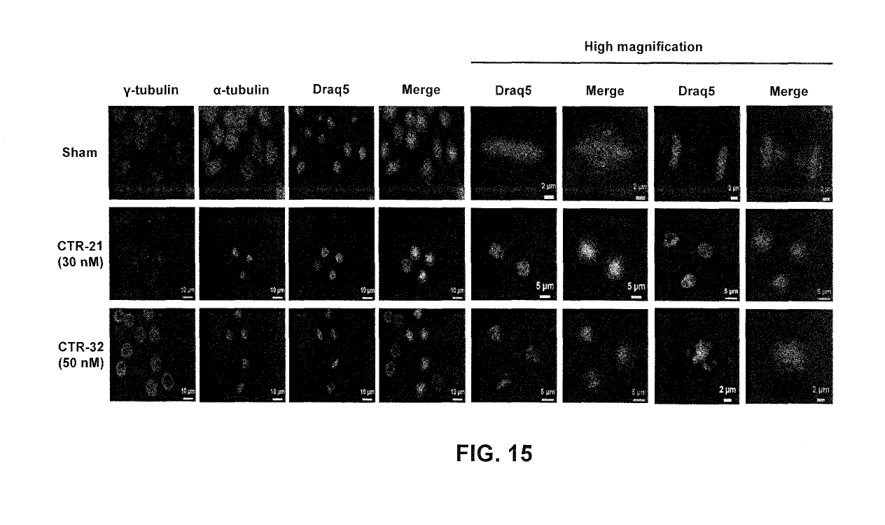

Data from cells immunostained with antibodies specific for α-tubulin or γ-tubulin showed that both CTR-21 and CTR-32 caused defects in the chromosome alignment at metaphase (FIG. 15). Asynchronous HeLa cells were treated with CTR-21 (30 nM) or CTR-32 (50 nM) for 12 hours, followed by staining DNA with Draq5 or immunestaining with antibodies specific for γ-tubulin or α-tubulin as shown on the top of the pictures. Chromosomes are not aligned properly at the center plate (white arrows).

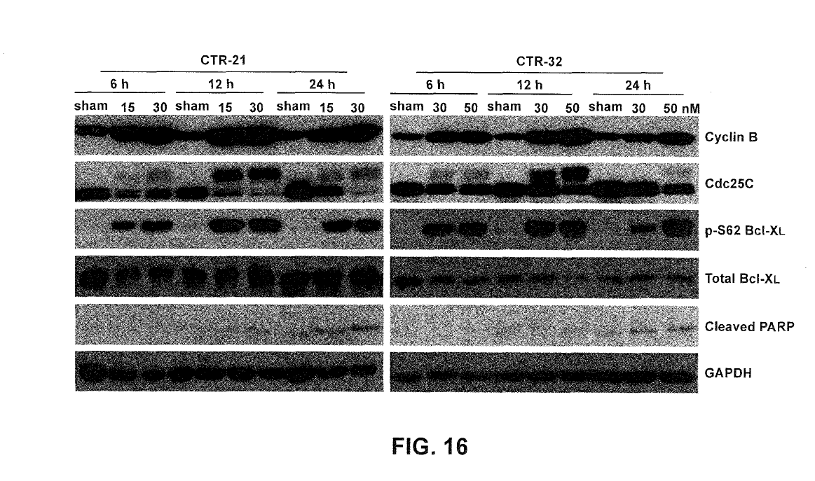

FIG. 16 shows that HeLa cells arrested at mitosis in the presence of CTR-21 or CTR-32 activated Bcl-X_{L} and apoptosis. Asynchronous HeLa cells were treated with CTR-21 (15 or 30 nM) or CTR-32 (30 or 50 nM) for 6, 12 or 24 hours (FIG. 16). The cell extracts of each sample were subjected to protein separation by SDS-PAGE, followed by Western blotting with antibodies specific for those listed at the right of gels pictures. High levels of cyclin B in the CTR treated samples as opposed to sham controls showed that the cells were arrested at M phase. The strong presence of high molecular weight Cdc25C (i.e., phosphorylated) at 12-hour post-treatment indicates that Cdk1/cyclin B was highly active by that time; thus, the cells already entered M phase. The treatment of cells with CTR-21 or CTR-32 caused the phosphorylation (i.e., “activation”) of the Bcl-X_{L} anti-apoptotic protein by 6-hour post-treatment. This was followed by cleavage of PARP proteins, suggesting that many cells underwent apoptosis by 24-hour post-treatment with CTR-21 (≥15 nM) or CTR-32 (≥30 nM).

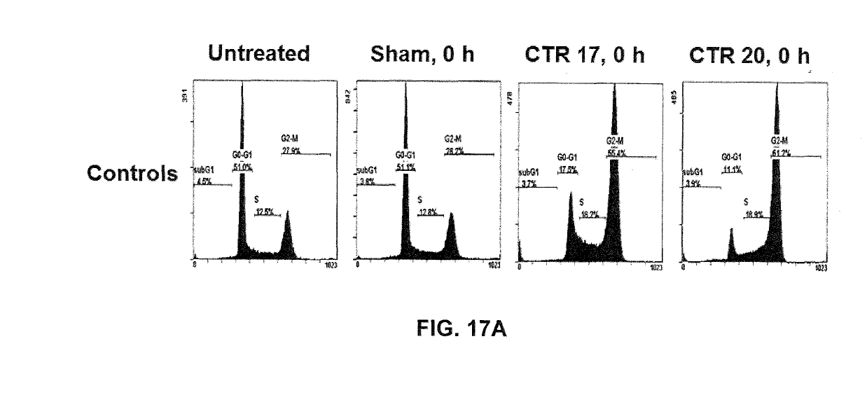

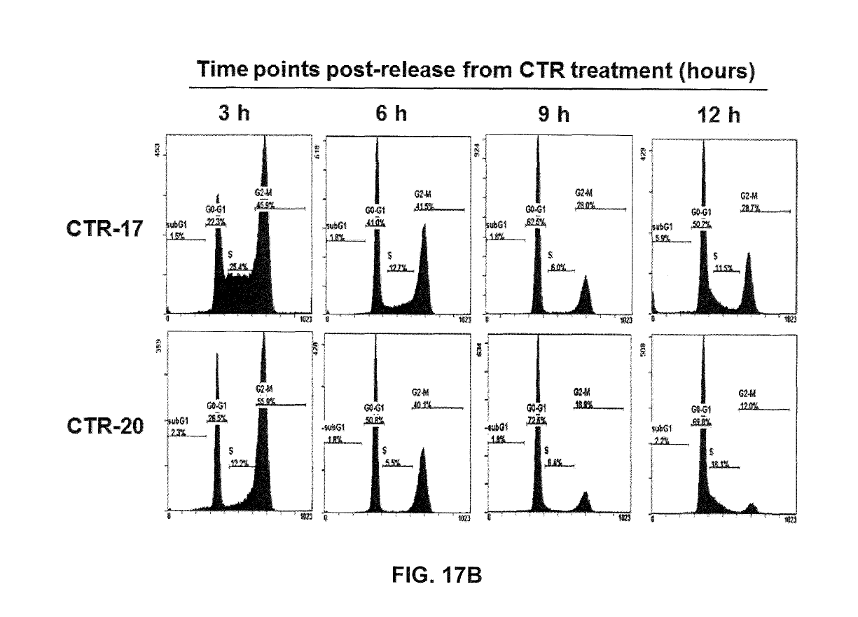

Mitotic arrest caused by CTR-17 and CTR-20 was reversible. FIG. 17A shows typical HeLa cell cycle histograms after they are treated with CTR-17 (3 μM; second from right) or CTR-20 (1 μM; far right) for 12 hours (which is defined as time 0 post-release). At time 0 post-release, cells were washed twice with 1×PBS, followed by re-suspension of the cells in 10 ml of pre-warmed, drug-free medium for durations of 3, 6, 9 or 12 hours (FIG. 17B). As can be seen from FIG. 17B, cells entered the cell cycle within 3 hours after CTR-17 and CTR-20 had been washed-off. This is in contrast to those cells still in culture medium containing CTR-17 and CTR-20 (FIGS. 11 and 13). Thus, this data shows that the effects of CTR-17 and CTR-20 are reversible.

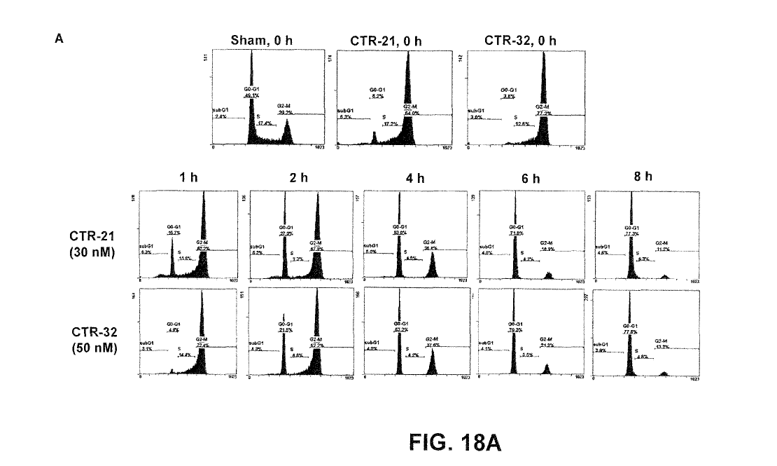

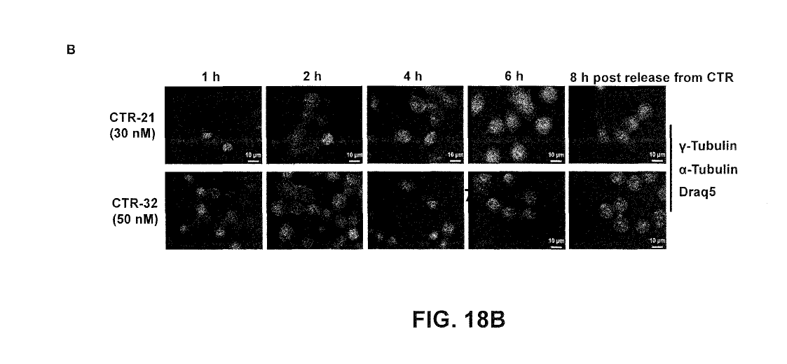

Like in the case of CTR-17 and CTR-20 (FIG. 17), the effect of CTR-21 and CTR-32 is reversible (FIG. 18). HeLa cells entered to G1 of the next cell cycle within 2 hours when those arrested at G2/M by the treatment of CTR-21 (30 nM) or CTR-32 (50 nM) for 12 hours were washed PBS, and then released into drug-free complete medium (FIG. 18A). Data from confocal microscopy (FIG. 18B) is consistent with the flow cytometry data (FIG. 18A). Together, our data appear to suggest that the CTR compounds may not have persistent side effects.

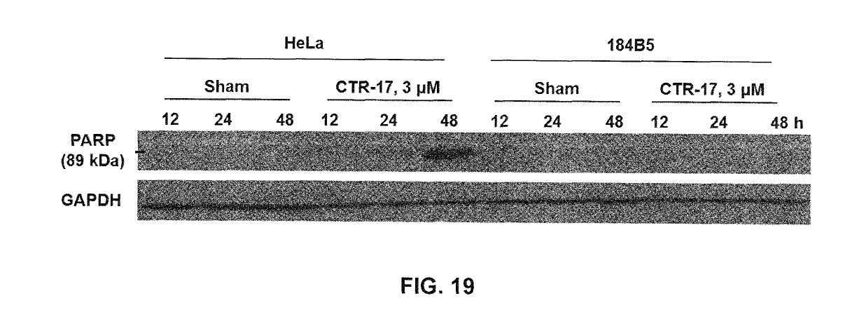

CTR-17 induces apoptosis in a cancer cell-specific manner (FIG. 19). Western blot analysis was carried out with an anti-PARP antibody at time points of 12, 24 or 48 hours post-treatment, using whole cell extracts prepared from asynchronous HeLa cells. As can be seen from FIG. 19, CTR-17 at 3.0 μM induced apoptosis by 48-hour post-treatment in HeLa cells but not in 184B5 non-cancer cells. This data is consistent with the data shown in Table 2.

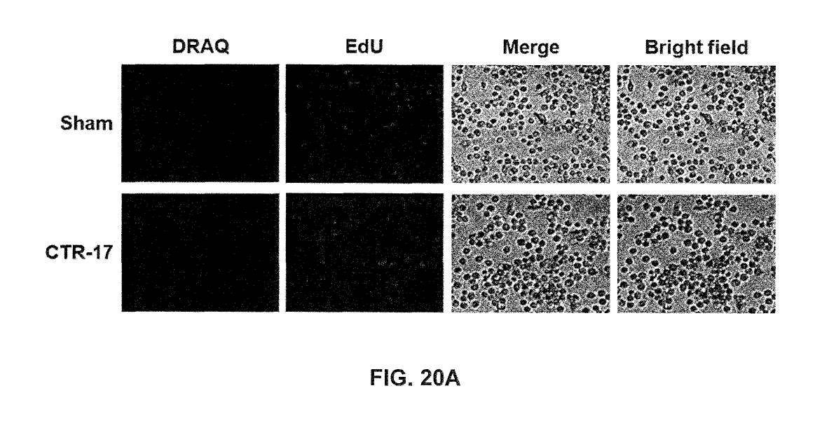

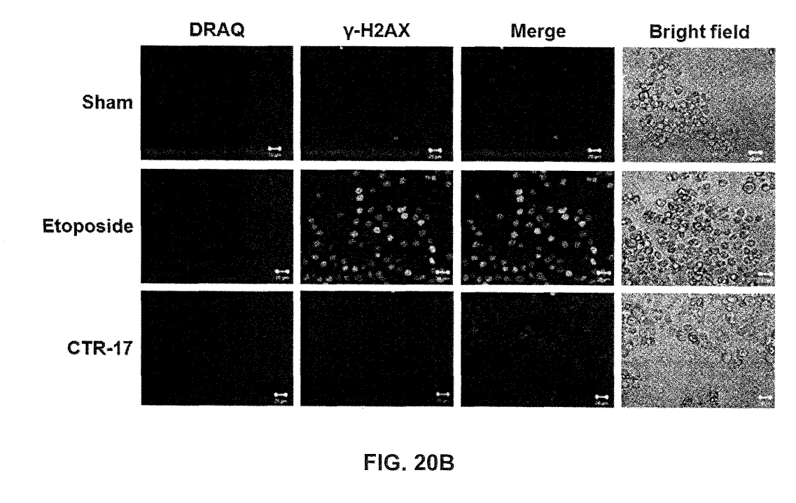

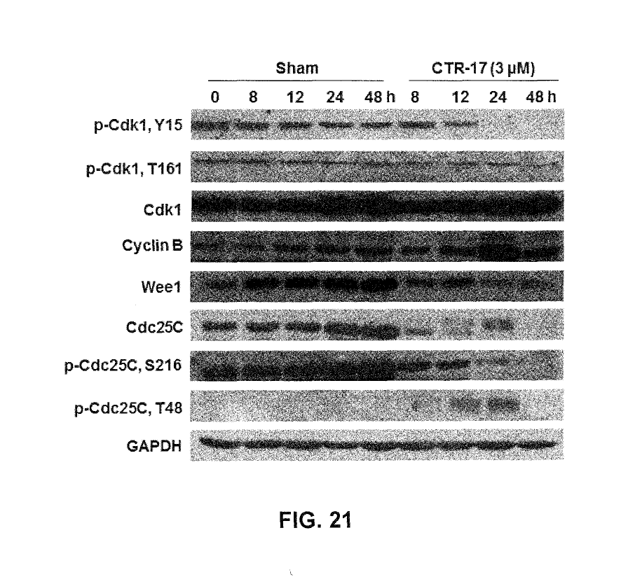

CTR-17 caused neither impediment of DNA replication nor DNA damage. FIG. 20A shows asynchronous HeLa cells treated with CTR-17 (3.0 μM) for 24 hours, then fed with EdU (10.0 μM) for 1 hour immediately prior to harvesting them for analysis. The detection of EdU incorporated into DNA was carried out by fluorescence microscopy. FIG. 20B shows cell immunostaining with an antibody specific for γ-H2AX carried out to detect damaged DNA (i.e., damage repairing). Etoposide (50.0 μM) was used as a positive control. As can be seen from the exemplary images in FIG. 20, under the experimental conditions used, EdU positive cells were 25.4% and 22.0% for the sham control and CTR-17 (3.0 μM) treated cells, respectively. This data thus indicates that CTR-17 does not cause any impediment on DNA replication. This data is consistent with the data shown in FIG. 14. Data presented in FIG. 20B demonstrates that CTR-17 does not cause any notable DNA damage.

Cells treated with CTR-17 arrested in mitosis, not in G2 (FIG. 21). A Western blot analysis was carried out with whole cell extracts prepared from asynchronously growing HeLa cells. Equal amounts of proteins were resolved by SDS-PAGE, and blotting was carried out with antibodies specific for those proteins listed at left of the gels shown in FIG. 21. Time points in hours (h) are post-treatment with 3.0 μM CTR-17. GAPDH was used as a loading control. “p-” denotes phosphorylation. The Western blot data shown in FIG. 21 demonstrated that CTR-17 causes cell cycle arrest in early M phase. This conclusion was derived from the fact that, judging from its phosphorylation on Tyr15, Cdk1 activity started to increase around 12-hour post-treatment and was fully active until 48 hours, the last time point examined. In an agreement with this conclusion, Cdc25C activity culminated around the same time points, suggesting, while not wishing to be limited by theory, that the amplification of Cdk1 activation circuit was in high gear at least until 24 hours post-treatment. However, Cdc25C was completely inactivated (see levels of protein and phosphorylation on Thr48) by 48 hours post-treatment, suggesting, while not wishing to be limited by theory, that Cdc25C is no longer needed to further amplify Cdk1 activity. Note that the initial conclusion of cell cycle arrest around G2/M by CTR-17 and CTR-20 was because data from flow cytometry was not detailed enough to make a definite conclusion whether cells were in the G2 or M phase.

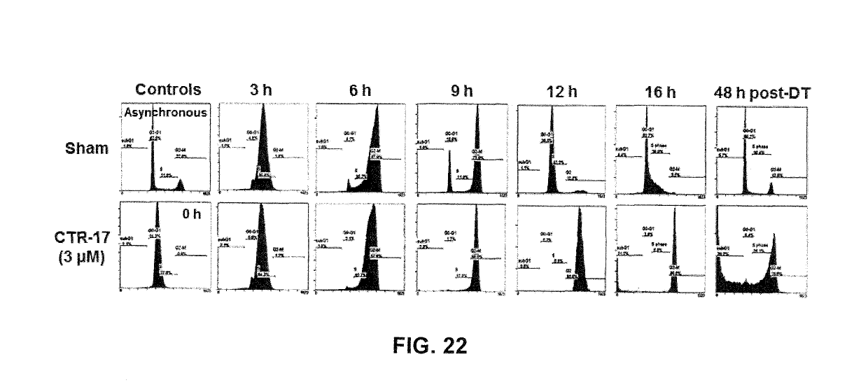

Cells did not exit mitosis in the presence of CTR-17. To accurately assess the effects of CTR-17 on cell cycle progression, HeLa cells synchronised at the G1/S border by double thymidine (DT) block were released into cell cycle in the absence (sham) or presence of CTR-17 (3.0 μM) for the duration in hours (h) indicated in FIG. 22. Cells treated with CTR-17 progressed through the cell cycle in a similar fashion with the sham-treated control until 9 hours post-release from the G1/S arrest by double thymidine treatment. Unlike the sham control, however, the treated cells did not exit M phase. Instead, as can be seen in FIG. 22, most of cells treated with CTR-17 eventually died by apoptosis without entering into the G1 phase of the next cell cycle (48 hours post-DT).

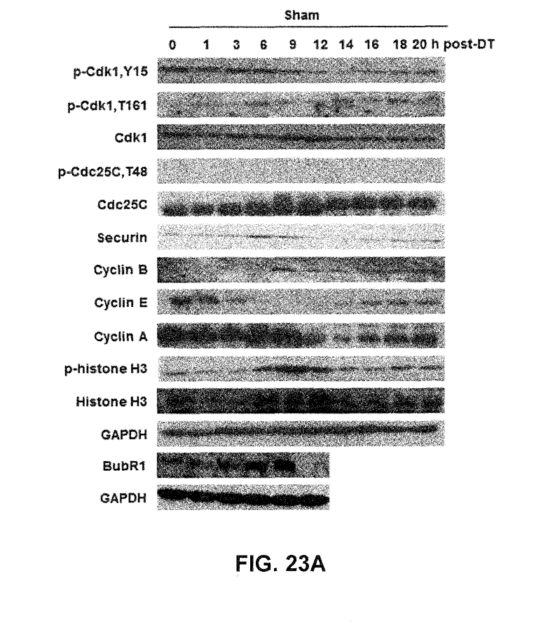

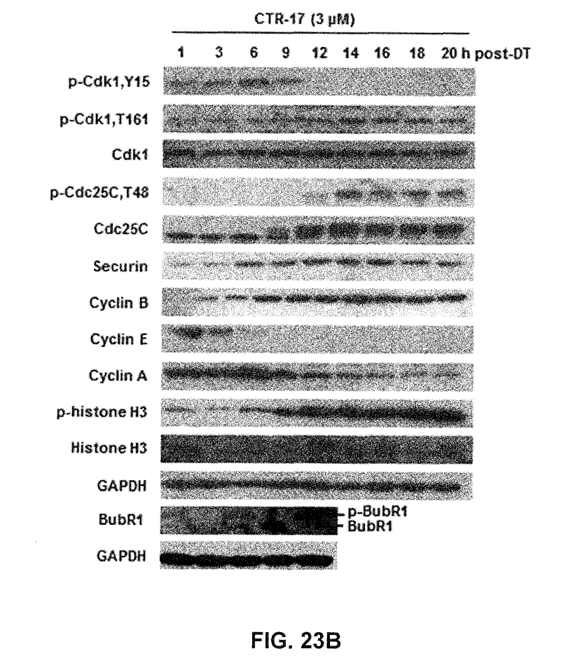

Data from the cell cycle study with synchronized cells demonstrated that CTR-17 arrests cells in early mitosis. HeLa cells synchronized at the G1/S border by double thymidine (DT) block were released into complete medium at time 0 in the absence (sham; FIG. 23A) or presence (FIG. 23B) of 3.0 μM CTR-17 for the duration (hours) indicated. Equal amounts of proteins were resolved by SDS-PAGE, followed by Western blotting with antibodies specific for proteins listed. “p-” denotes phosphoprotein. GAPDH was used as a loading control. Consistent with data from asynchronous cells (e.g., FIG. 21), CTR-17 arrested cells in early M phase when HeLa cells at the G1/S border were released into complete medium containing 3 μM CTR-17. Cdk1 and Cdc25C continue to be active in the presence of CTR-17, which is manifested by the dephosphorylation of Cdk1 on Tyr15 and phosphorylation of Cdc25C on Thr48, at least up to 20 hours post-release from double thymidine block. This coincided with the high levels of securin, cyclin B and histone H3 phosphorylation, while only negligible levels of cyclin E and cyclin A were observed. In addition, BubR1 was highly phosphorylated by 12 hours post-release. Together, while not wishing to be limited by theory, this data strongly suggest that the cell cycle was arrested in the presence of CTR at the spindle checkpoint step (prior to APC-mediated securin degradation), presumably due to the failing of proper alignment of chromosomes at the center plate. This conclusion is supported by other data presented, including FIGS. 2, 3, 4, 5, 11, 13, 14, 21, 22, and 25.

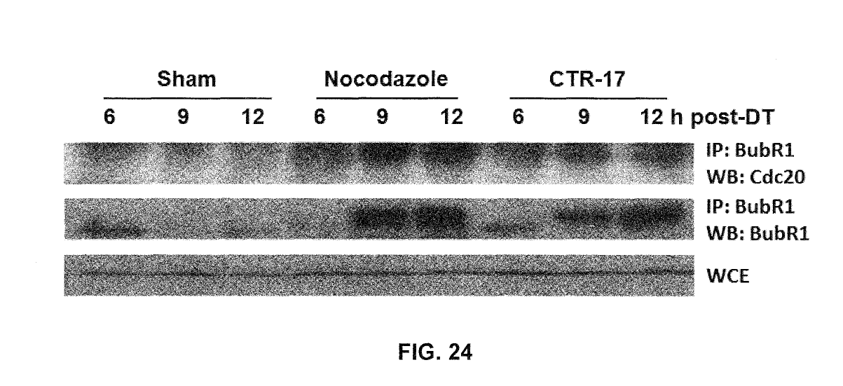

Co-immunoprecipitation confirmed that CTR-17 causes cell cycle arrest at the spindle checkpoint activation step. HeLa cells synchronised at the G1/S boundary by double thymidine (DT) block were untreated (sham), treated with 20 ng/ml nocodazole, or treated with 3.0 μM CTR-17 for the duration (h denotes hour(s)) indicated in FIG. 24. Total protein extracts were subjected to immunoprecipitation with an anti-BubR1 antibody, followed by protein separation by SDS-PAGE and Western blotting with an anti-Cdc20 antibody to examine the interaction between BubR1 and Cdc20. Consistent with data from flow cytometry and Western blotting, the data from co-immunoprecipitation (FIG. 24) demonstrated that CTR-17 arrested cells at the spindle checkpoint step as BubR1 and Cdc20 were associated in the presence of CTR-17. However, APC was not yet active. The cell cycle arrest point by CTR-17 is similar to that by nocodazole.

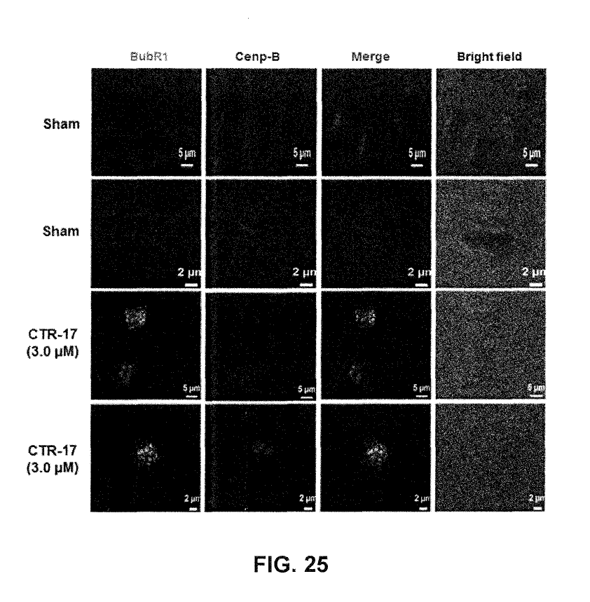

BubR1 accumulated at the kinetochore in the presence of CTR-17. Asynchronously growing HeLa cells were sham-treated or treated with CTR-17 (3.0 μM) for 12 hours, fixed, and then immunostained with antibodies specific for BubR1 or Cenp-B (centromere staining). As can be seen from FIG. 25, the accumulation of BubR1 at the kinetochore indicates the lack of proper tension between the kinetochore and the mitotic spindle/centrosome and, thus, perpetually extending the activity of spindle assembly checkpoint.

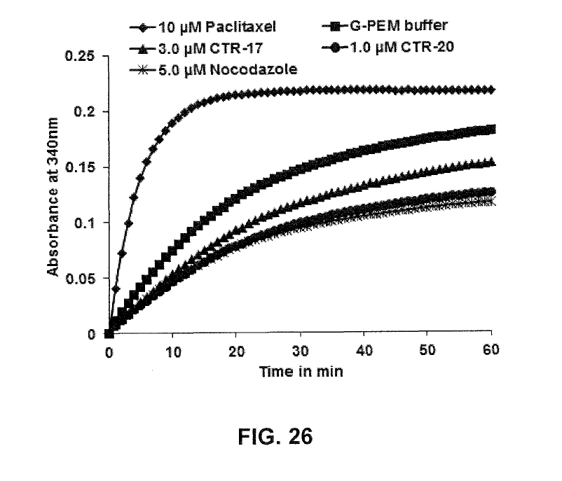

Both CTR-17 and CTR-20 inhibited tubulin polymerization. Purified porcine tubulin and 1.0 mM GTP were added to a reaction mixture containing 10.0 μM paclitaxel, 3.0 μM CTR-17, 1.0 μM CTR-20, or 5.0 μM nocodazole. Polymerization of tubulin was monitored every minute for one hour at 340 nm and 37° C. by spectrophotometry (FIG. 26). CTR-17 and CTR-20 caused an extended growth phase and took a long time to achieve steady state equilibrium in the microtubule polymerization reaction. This pattern is similar to that of nocodazole but different from that of paclitaxel, a microtubule stabilizing agent. While not wishing to be limited by theory, this data thus indicates that CTR-17 and CTR-20 are inhibitors of tubulin polymerization.

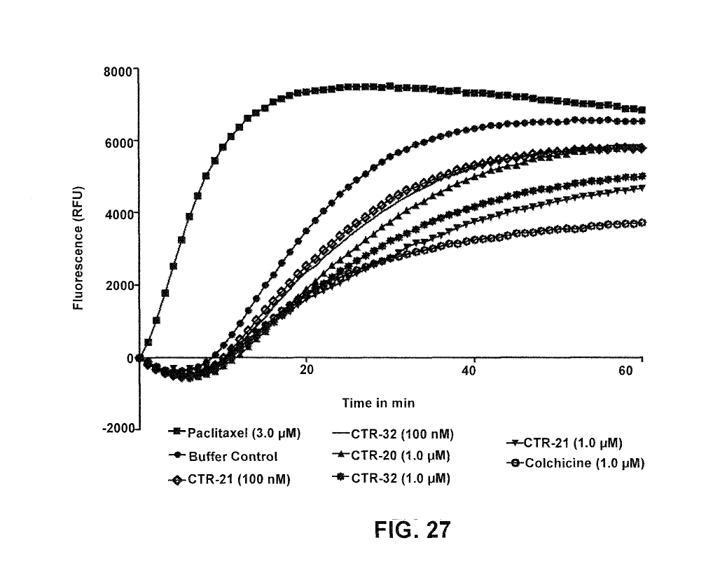

Both CTR-21 and CTR-32 are microtubule polymerization inhibitors. Data shown in FIG. 27 is from an in vitro microtubule assembly assay to examine if CTR-21 and CTR-32 were microtubule inhibitors similarly to CTR-17 and CTR-20. Microtubule polymerization was monitored in relation to the incorporation of fluorescent reporter molecules into microtubules. The assay was carried out for one hour at 37° C., reading one minute intervals by spectrophotometry. CTR-21 and CTR-32 were used at two different concentrations, 100 nM and 1.0 μM. At 10 nM, both of them inhibited microtubule polymerization to a similar degree of CTR-20 at 1.0 μM, indicating that CTR-21 and CTR-32 are stronger microtubule inhibitors than CTR-20. Among the two, CTR-21 appears to be more effective than CTR-32 in inhibiting microtubule polymerization.

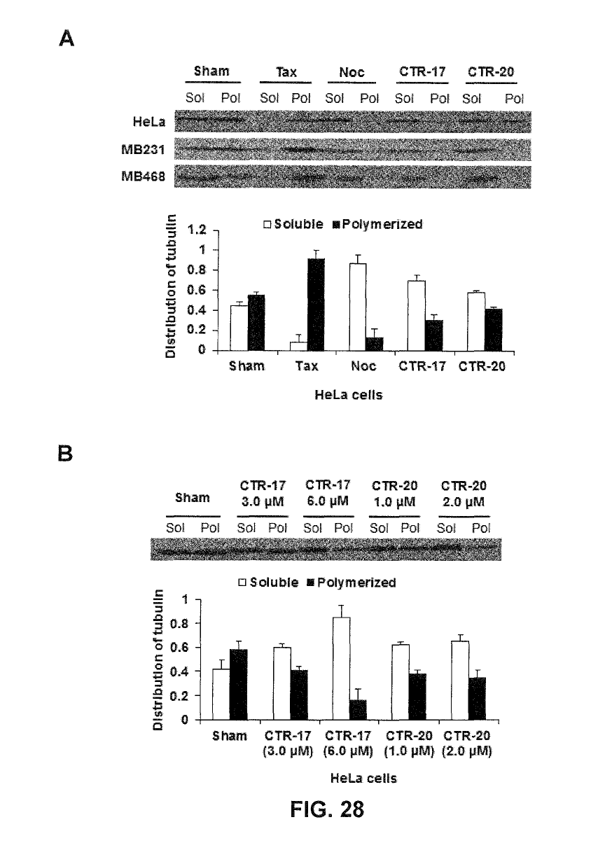

CTR-17 and CTR-20 decreased the polymerized pool of tubulin (FIG. 28). HeLa, MDA-MB-231 and MDA-MB-468 cells were sham-treated, treated with 50.0 nM paclitaxel (Tax), 50.0 ng/ml nocodazole (Noc), 3.0 μM CTR-17, or 1.0 μM CTR-20 for 12 hours. Cell lysates were separated into polymerization (Pol) and soluble (Sol) fractions, and equal amounts of proteins were resolved by SDS-PAGE, followed by immunoblotting with an antibody specific for α-tubulin. Bands (upper panel of FIG. 28A) were quantified with densitometry and expressed in a graph form (lower panel of FIG. 28A). The sum of soluble and polymerized fractions is 1.0. HeLa cells treated with different concentrations of CTR-17 or CTR-20 were subjected to fractionation and immunoblotting as described for FIG. 28A. Both CTR-17 and CTR-20 reduced the tubulin polymer fraction similarly to nocodazole. This fractionation pattern is in a stark contrast with paclitaxel, which increases the tubulin polymer fraction. The reduction of tubulin polymerization by CTR-17 was dose-dependent within the range of concentrations of 3-6 μM (FIG. 28B). However, the effect of CTR-20 was already saturated at the 1.0 μM concentration, indicating that CTR-20 is a stronger inhibitor of tubulin polymerization (FIG. 28B).

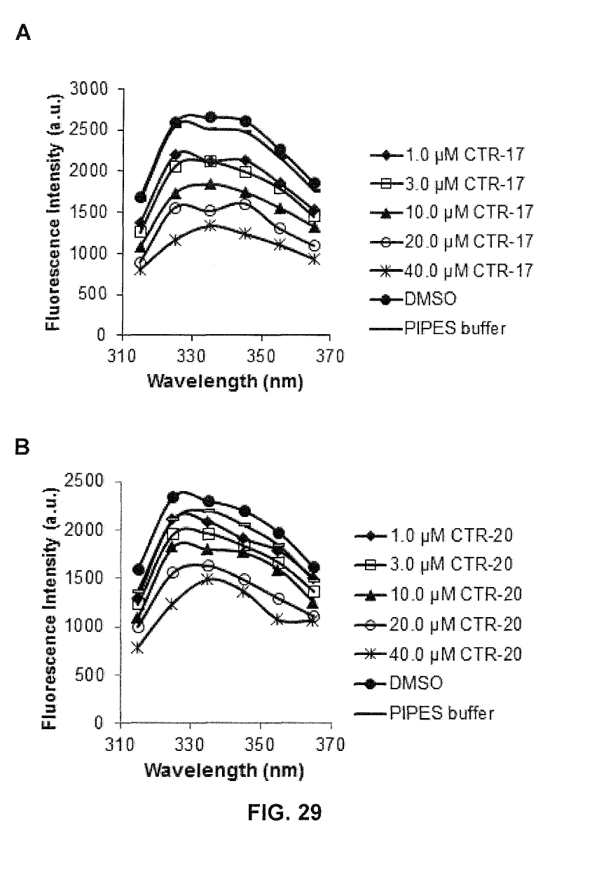

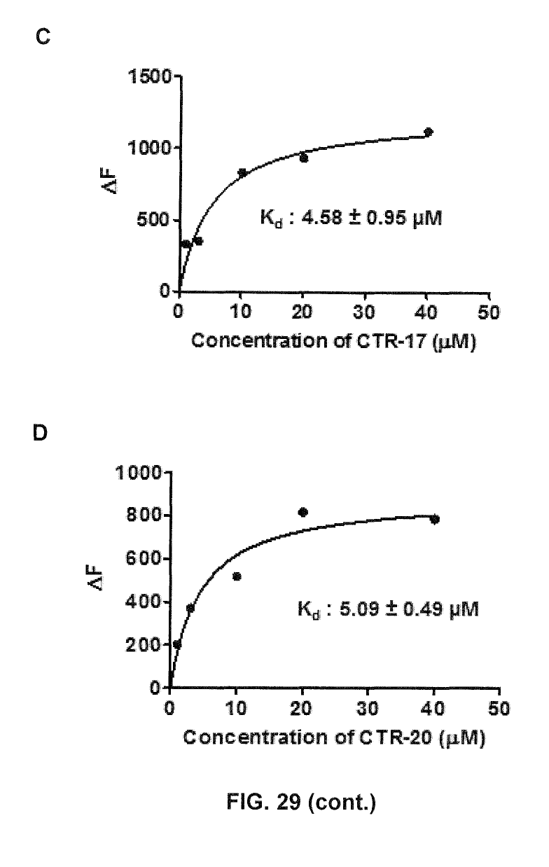

Both CTR-17 and CTR-20 bound to tubulin. As can be seen from FIGS. 29A and 29B, respectively, CTR-17 and CTR-20 quenched the intrinsic tryptophan fluorescence of tubulin in a dose-dependent manner. Purified tubulin dissolved in 25 mM PIPES buffer was incubated in the presence or absence of different concentrations of CTR-17 or CTR-20 for 30 minutes at 37° C. Fluorescence was monitored by excitation of the reaction mixture at 295 nm, and the emission spectra were recorded from 315 to 370 nm. FIGS. 29C and 29D, respectively, show the change in fluorescence intensity plotted against the drug concentrations of CTR-17 and CTR-20 to determine the dissociation constant. ΔF (y-axis) is the change in fluorescence intensity of tubulin when bound by the CTR compounds. Data are an average of five independent experiments. Both CTR-17 and CTR-20 bound to tubulin, in a dose-dependent manner.

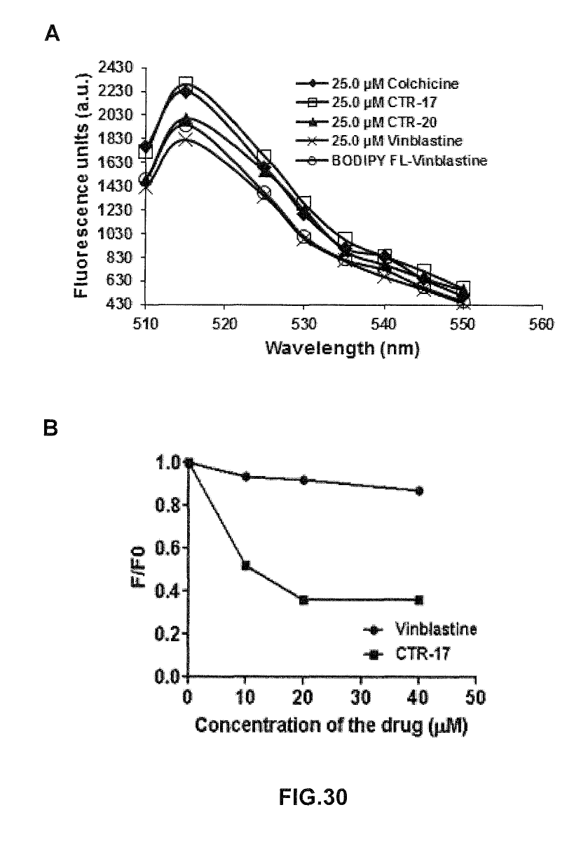

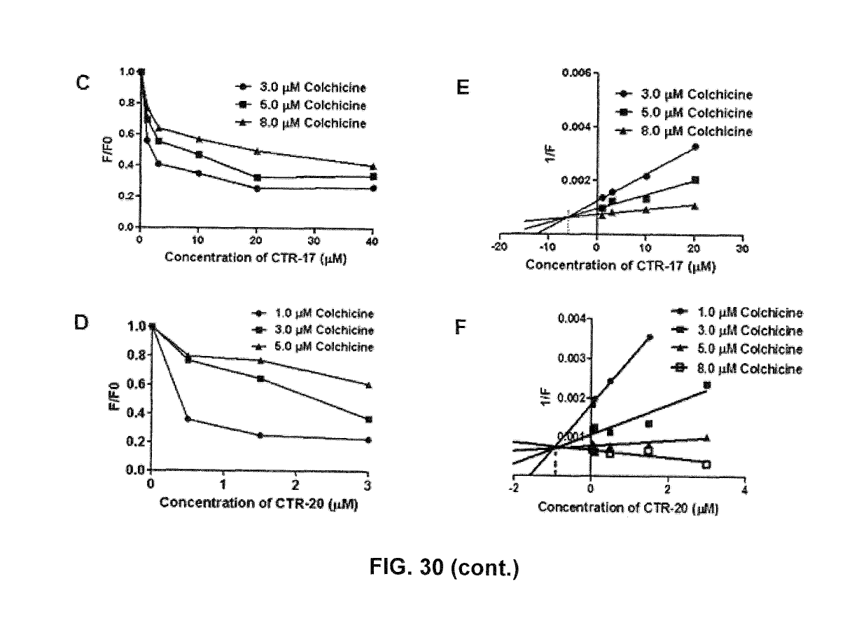

Both CTR-17 and CTR-20 inhibited the binding of colchicine to tubulin (FIG. 30). The CTR compounds tested, similar to colchicine, did not bind to the vinblastine binding site on the tubulin. 25 μM each of colchicine, CTR-17, CTR-20, or vinblastine was incubated with tubulin for 1 hour to promote the formation of complexes between tubulin and each of these compounds. The resultant complexes were incubated for 30 minutes with 5 μM of the fluorescent BODIPY FL-vinblastine to determine if the binding of each compound to tubulin was in competition with vinblastine. CTR-17 binds to tubulin at or near the colchicine-binding site. The tubulin-fluorescent colchicine complex was incubated with increasing concentrations of either vinblastine or CTR-17. CTR-17 but not vinblastine competed with (fluorescent) colchicine. The CTR compounds depressed the fluorescence of the colchicine-tubulin complex in a dose-dependent manner. Tubulin was incubated with different concentrations of CTR-17 or CTR-20 for 1 hour, in three separate sets with different concentrations of colchicine as indicated in FIG. 30. Inhibitory constants of CTR-17 and CTR-20 were determined. The fluorescence intensity of the final tubulin complex (FIGS. 30C and 30D) was used to determine the inhibitory concentration (Ki) utilizing a modified Dixon plot (FIGS. 30E and 30F). In FIG. 30, F is the fluorescence of the complexes of CTR-17 (or CTR-20)-colchicine-tubulin or vinblastine-colchicine-tubulin complex, and F0 is the fluorescence of the colchicine-tubulin complex. Data are average of at least four independent experiments. The data of FIG. 30, while not wishing to be limited by theory, suggests that the binding sites of both CTR-17 and CTR-20 on the tubulin may overlap with that of colchicine but not that of vinblastine.

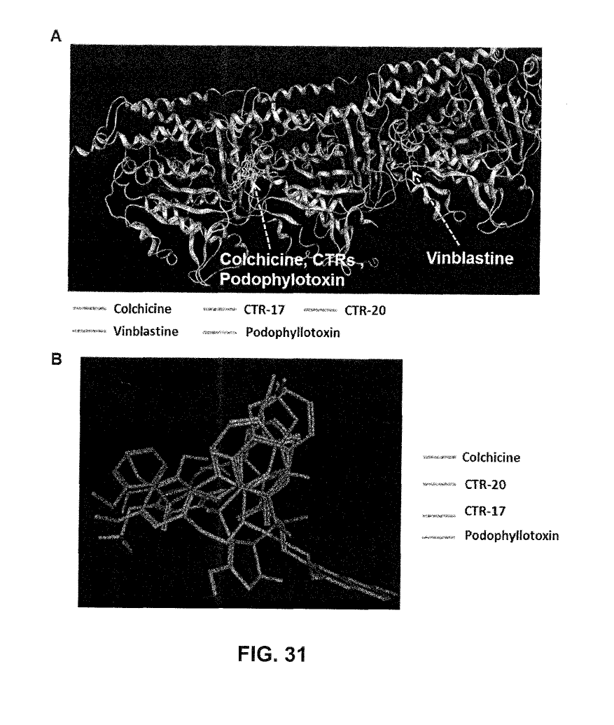

FIG. 31A shows the result of molecular docking predicting that the tubulin-binding sites of colchicine (blue in color image; medium grey in FIG. 31A), CTR-17 (green in color image; lighter grey in FIG. 31A), CTR-20 (magenta in color image; darker grey in FIG. 31A) and podophyllotoxin (yellow in color image; light grey in FIG. 31A) were very close, but not with that of vinblastine (red in color image; darkest grey in FIG. 31A). The 3D X-ray structure of tubulin (PDB code: 1 SA0) was used in this study. FIG. 31B shows the chemical structures of colchicine (blue in color image; darkest grey in FIG. 30B), CTR-17 (green in color image; light grey in FIG. 31B), CTR-20 (magenta in color image; dark grey in FIG. 31B) and podophyllotoxin (light red in color image; lightest grey in FIG. 31B) are shown to aid the visualization of the close overlap when bound to their respective binding sites on the tubulin. In sum, while not wishing to be limited by theory, data from molecular modeling showed that the tubulin sites bound by CTR-17 and CTR-20 essentially overlap with those of colchicine and podophyllotoxin, but are completely different from that of vinblastine.

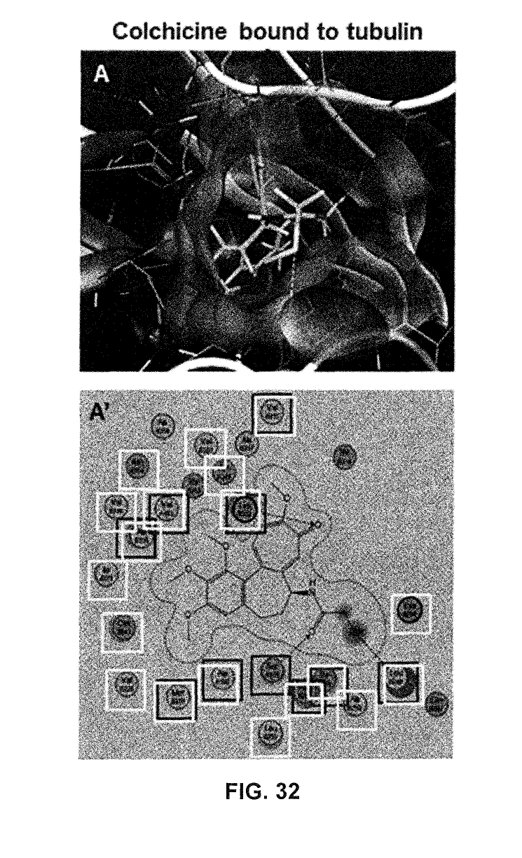

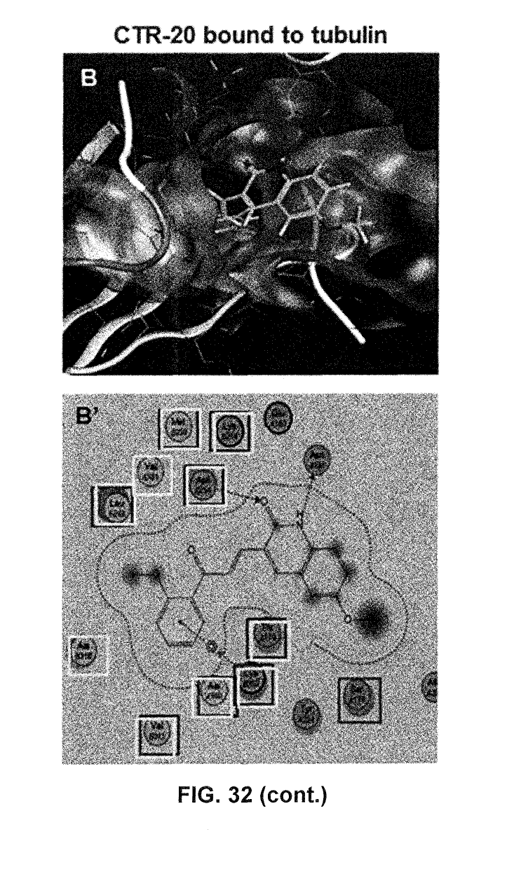

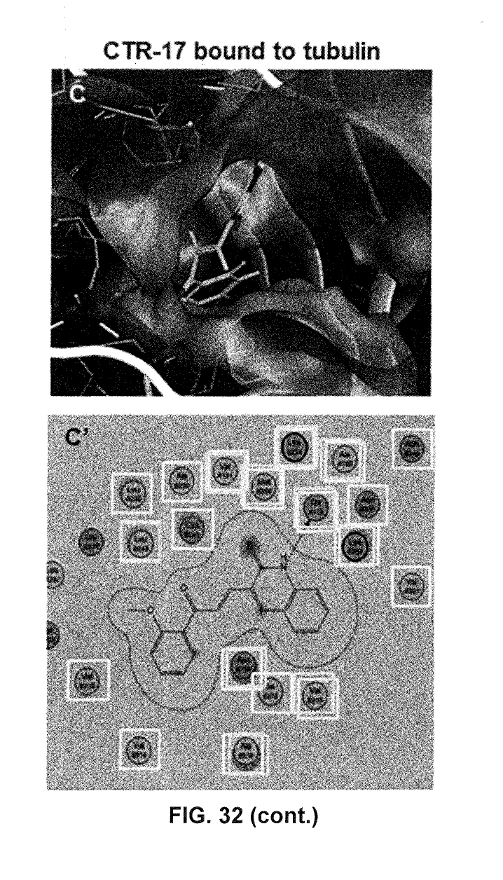

The predicted interaction between the tubulin heterodimer (PDB code: 1SA0) and colchicine (A), CTR-20 (B), and CTR-17 (C) is shown in a 3D pattern in FIG. 32. 2D ligand interaction diagrams in FIG. 32 show potential chemical interactions between amino acids and compounds within a distance of 4 Å to colchicine (A′), CTR-20 (B′), or CTR-17 (C′). There are three H-bonds between tubulin and colchicine, while one and two H-bonds between tubulin-CTR-17 and tubulin-CTR-20, respectively. The direction of arrows shows the electron donor in hydrogen bonding. H-bonds are formed through a side chain and an amino acid backbone, respectively. A number of hydrophobic and polar residues overlap between colchicine and the CTR compounds in binding to tubulin. The color codes are: dark grey (red in a color image) square boxes are tubulin amino acids that are common in binding to colchicine and CTR-20; lightest grey (yellow in a color image) boxes are those common in binding to colchicine and CTR-17; and light grey (blue in a color image) boxes are those common in binding to CTR-17 and CTR-20. Non-covalent and Van der Waals interactions stabilize the binding between tubulin and these compounds. In sum, data obtained from molecular modeling showed that the binding mode of colchicine, CTR-17 and CTR-20 to tubulin is very similar as they often bind to the same amino acid residues on tubulin. However, they also show differences in the binding mode. For example, colchicine forms three H-bonds, and CTR-17 and CTR-20 form only one and two H-bonds, respectively. While not wishing to be limited by theory, it is possible that these differences are directly relevant, for example, to efficacy, toxicity and reversibility of the compounds.

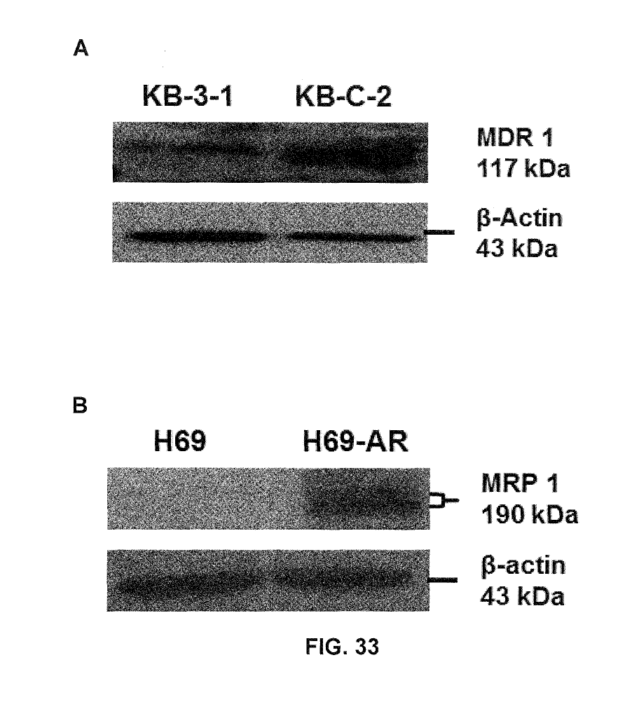

CTR-17 and CTR-20 are effective against multidrug-resistant cancer cells. Western blotting of whole cell extracts prepared from the parental KB-3-1 and MDR1-overexpressing KB-C-2 isogenic cell lines are shown in FIG. 33A. Western blotting of whole cell extracts prepared from the parental H69 and MRP1-overexpressing H69AR isogenic cell lines are shown in FIG. 33B. The data in Table 7 show that CTR-17 and CTR-20 kill parental (KB-3-1) and MDR1-overexpressing multidrug-resistant cells (KB-C-2) with a similar potency, and both of these CTR compounds preferentially kill MRP1-overexpressing multidrug-resistant cells (H69AR) over matching small cell lung cancer cells (SW-1271). An SRB assay was used to determine the anti-proliferation effects. Colchicine, paclitaxel and vinblastine are largely ineffective in killing multidrug-resistant cells. Data shown in FIG. 33 and Table 7 demonstrate that both CTR-17 and CTR-20 kill KB-3-1 (cervical carcinoma) and its MDR1-overexpressing multidrug-resistant isogenic cells (KB-C-2) with similar potency, while colchicine, paclitaxel, and vinblastine kill the drug-resistant cells at least 10 fold less effectively than non-resistant cells. Furthermore, both of the CTR compounds preferentially killed the MRP1-overexpressing, multidrug-resistant H69AR cells over a matching non-cancer small cell lung cancer cell line (SW-1271).

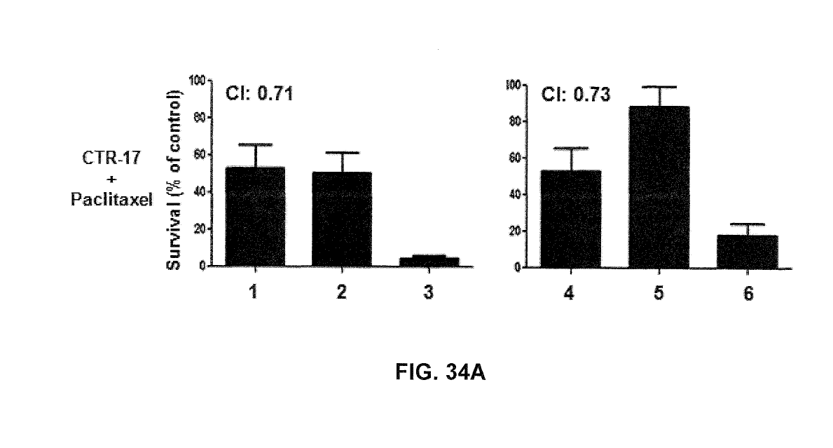

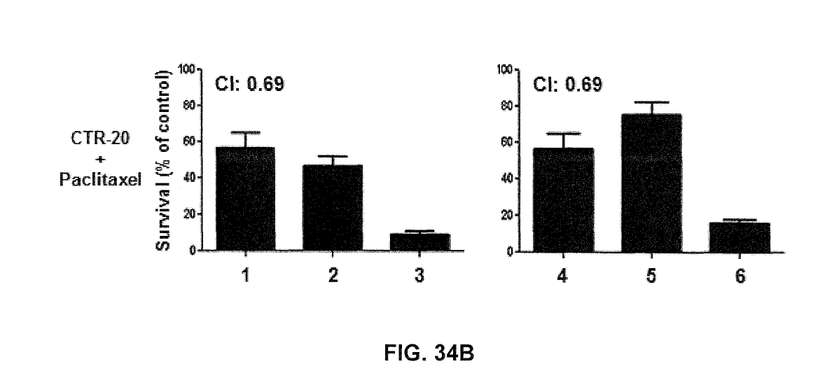

As can be seen from FIG. 34, both CTR-17 and CTR-20 show synergistic effects when combined with paclitaxel. The KB-C-2 multidrug-resistant cells were subjected to an antiproliferation study with combinations of different doses of CTR compounds and paclitaxel. Data from SRB assays was used to construct a sigmoidal dose-response curve, from which the median effect dose (Dm), fraction affected (Fa) and slope of the curve (m) were determined. These values were then used to determine the combination effect between CTR-17/-20 with paclitaxel, as outlined in the methodology section. Part of the CTR-17 data presented in Table 8 is shown in a graph form in FIG. 34A. Lanes denote: 0.65 μM CTR-17 (lanes 1 & 4), 23 nM paclitaxel (lane 2), 0.65 μM CTR-17 plus 23 nM paclitaxel (lane 3), 5.75 nM paclitaxel (lane 5), and 0.65 μM CTR-17 plus 5.75 nM paclitaxel. Part of CTR-20 data presented in Table 8 is shown in a graph form in FIG. 34B. Lanes denote: 0.25 μM of CTR-20 (lanes 1 & 4), 23 nM paclitaxel (lane 2), 0.25 μM CTR-20 plus 23 nM paclitaxel (lane 3), 11.5 nM paclitaxel (lane 5), and 0.25 μM CTR-20 plus 11.5 nM paclitaxel (lane 6). CI denotes combination index. CI<1.0, CI=1.0 and CI>1.0 are synergistic, additive and antagonistic, respectively (Chou, 2006). For more detail, see Table 8. Data presented are mean±S.E.M value of triplicates of at least four independent experiments. As seen from the data in FIG. 34 and Table 8, both CTR-17 and CTR-20 showed synergistic cell killing effects when used in combination with paclitaxel on KB-C-2 multidrug-resistant cells. Note that the combination index (CI) of 0.65 μM CTR-17 and 23.0 nM paclitaxel was 0.71±0.08, and that of 0.25 μM CTR-20 and 11.5-23.0 nM of paclitaxel was 0.69. Thus, the combination of CTR compounds and paclitaxel can be substantially synergistic on the MDR1-overexpressing multidrug-resistant KB-C-2 (and, while not wishing to be limited by theory, other) cells.

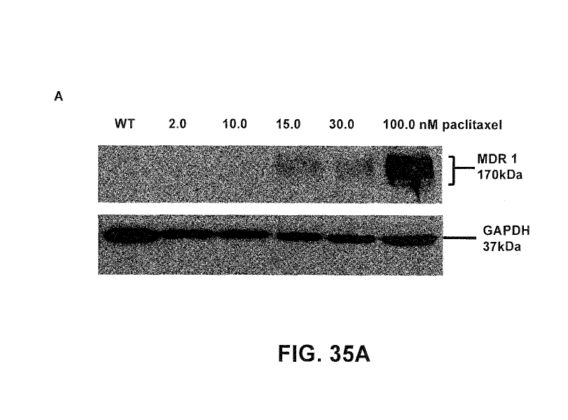

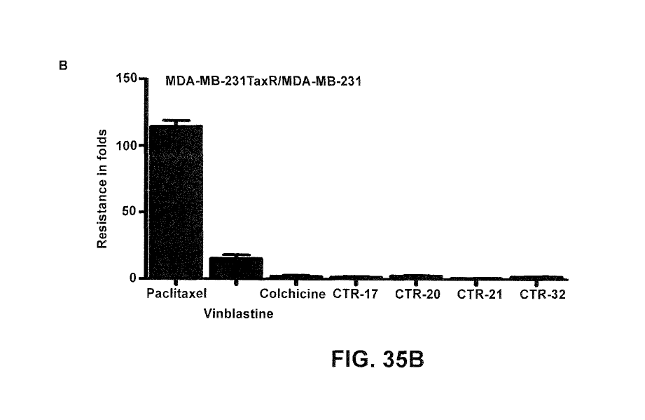

Data in FIG. 35 shows that the MDR1-overexpressing paclitaxel-resistant MDA-MB231TaxR is sensitive to CTR-17, CTR-20, CTR-21 and CTR-32. The paclitaxel-resistant MDA-MB231TaxR cell line was generated in house by culturing the triple-negative MDA-MB231 metastatic breast cancer cell line in the incrementally increased concentrations of paclitaxel over one-year period, until the cells grow and proliferate in the medium containing 100 nM of paclitaxel. Subsequently, the cells were dosed with 100 nM paclitaxel once every month and removed from the drug at least for one passage before being used for an experiment. FIG. 35A shows the Western blotting of MDA-MB231TaxR cells at different levels of paclitaxel resistance alongside the MDA-MB231 parental cells (WT) for the expression of P-glycoprotein (MDR1): 2.0, 10.0, 15.0, 30.0 and 100.0 nM are cells selected at the concentrations of paclitaxel at 2.0, 10.0, 15.0, 30.0 and 100.0 μM, respectively. Parental MDA-MB231 cells (WT) do not express P-glycoprotein; however, the level of P-glycoprotein expression increases with increasing levels of resistance in the TaxR cells (FIG. 35A). Data in FIG. 35B shows that colchicine, CTR-17, CTR-20, CTR-21, and CTR-32 kill MDA-MB231TaxR cells (selected in 100.0 nM paclitaxel) to the same degree as the parental MDA-MB231 cells. However, MDA-MB231TaxR cells were resistant to paclitaxel and vinblastine by approximately 114 and 15 folds, respectively, suggesting that MDA-MB231TaxR cells are multidrug resistance in nature.

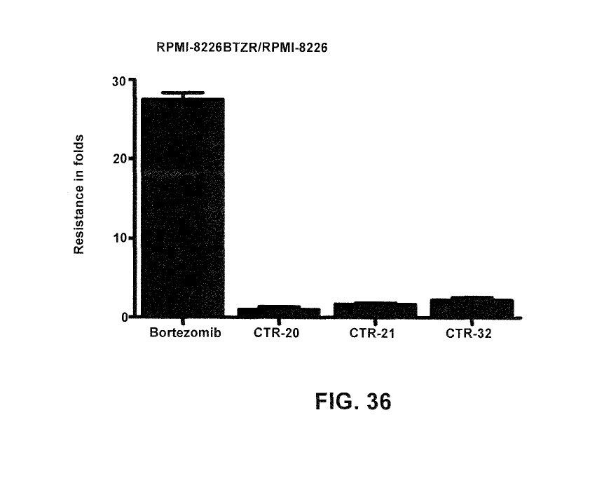

Data in FIG. 36 shows that the bortezomib-resistant RPMI-8226BTZR multiple myeloma cells are sensitive to CTR-20, CTR-21 and CTR-32. The bortezomib-resistant RPMI-8226BTZR cell line was developed in house by culturing the RPMI-8226 multiple myeloma cell line in the gradually increased concentrations of bortezomib, a proteasome inhibitor targeting β5 peptide of the 20S catalytic subunit. The RPMI-8226BTZR over-expresses β1, β2 and β5 peptides of the 20S subunit. FIG. 36 shows that RPMI-8226BTZR is approximately 28-fold more resistant to bortezomib, compared to the RPMI-8226 parental cells. However, RPMI-8226BTZR is sensitive to CTR-20, CTR-21 and CTR-32 (FIG. 36).

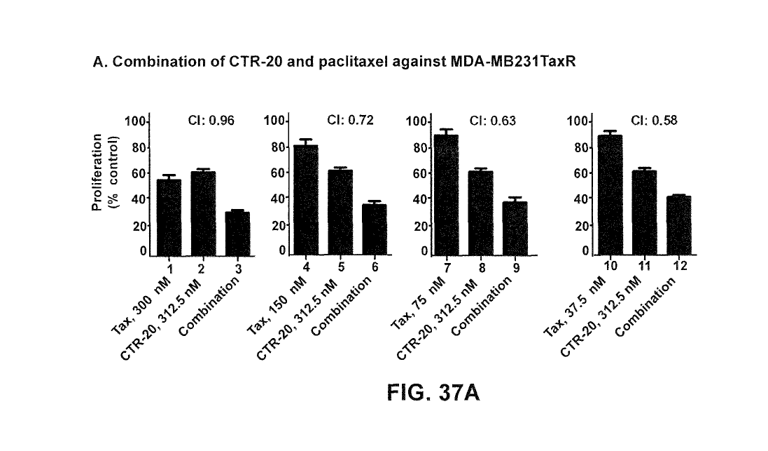

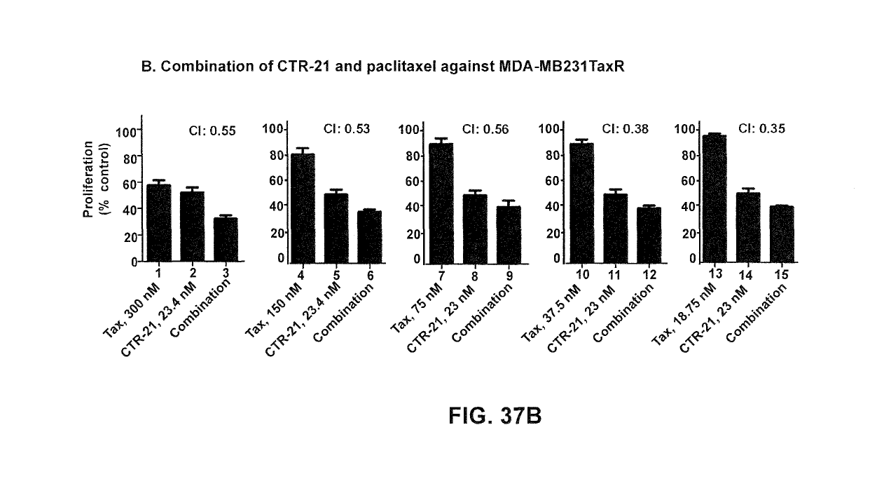

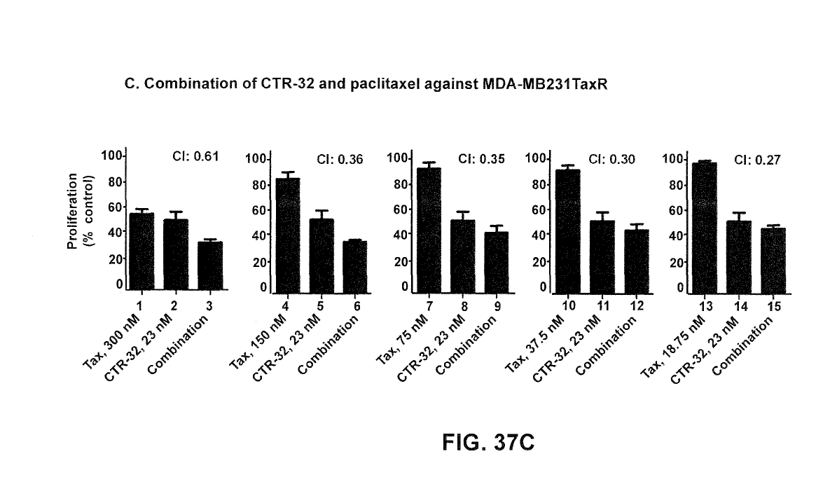

When combined with paclitaxel, CTR-20, CTR-21 and CTR-32 showed synergy in killing the paclitaxel/multidrug-resistant MDA-MB231TaxR cells (FIG. 37 and Table 9). It was found previously that both CTR-17 and CTR-20 are synergistic with paclitaxel in killing the MDR1-overexpressing KB-C-2 cells (FIG. 34 and Table 8). Data in FIG. 37 and Table 9 show the combinational effects of paclitaxel and CTR-20 (A), CTR-21 (B) and CTR-32 (C) against MDA-MB231TaxR cells. For this set of experiments, we combined 4-5 different doses of paclitaxel with a single dose of CTR-20, CTR-21 or CTR-32. Cell viability was always greater than 50% when each of these drugs was used singly at the doses used. However, the cell viability reduced at the same doses when combined with paclitaxel and CTR-20, CTR-21 or CTR-32. For example, the combination of 0.3 μM of CTR-20 and 37.5 nM paclitaxel killed MDA-MB231TaxR cells approximately 56% (Table 9). The combination of 23.4 nM of CTR-21 and 18.75 nM paclitaxel killed MDA-MB231Tax cells 58% (Table 9). Finally, the combination of 23.4 nM of CTR-32 and 18.75 nM paclitaxel killed 52% of MDA-MB231TaxR cells (Table 9). These data are translated into combinational index (CI) of 0.59, 0.35 and 0.27, respectively (Table 9 and FIG. 37), showing that these combinations are synergistic. Since paclitaxel is generally toxic, the synergistic effects of paclitaxel-CTR combinations at low drug concentrations against multidrug-resistant cancer cells will provide new opportunities of controlling drug-resistant cancer with low side effects.

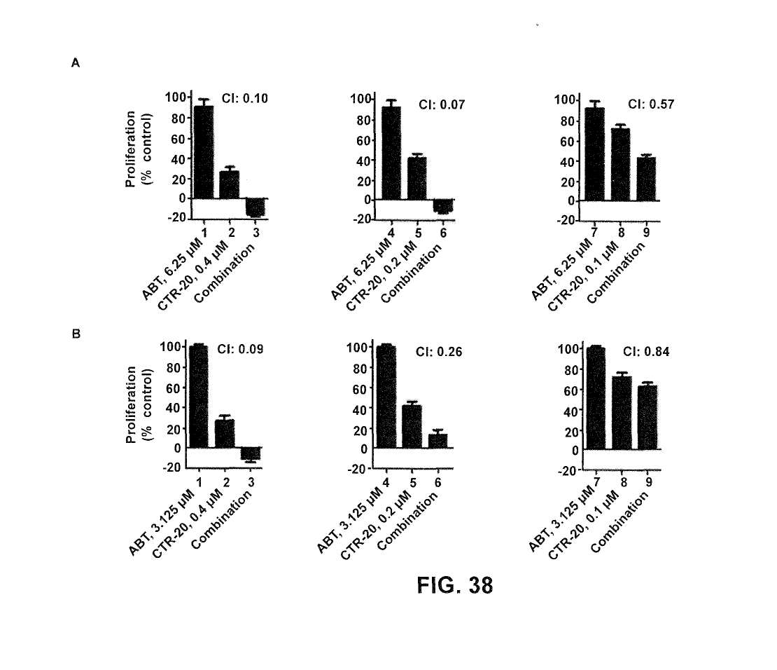

Data in FIG. 38 and Table 10 show that the combination of CTR-20 and ABT-737 is synergistic against MDA-MB231 triple-negative metastatic breast cancer cells. We examined the combinational effects CTR-20 and the inhibitor of anti-apoptotic Bcl2 family proteins. Data from all of the different combinations by three different doses of CTR-20 and two different doses of ABT-737 showed synergistic effects on MDA-MB231 cells. In particular, the combination of 0.2 μM of CTR-20 and 6.25 μM of ABT-737 showed the CI value of 0.07, a high degree of synergism. Similarly, the CI of the 0.4 μM of CTR-20 and 6.25 μM of ABT-737 combination was 0.10. At these combinations, MDA-MB231 cell population was eliminated completely (Table 10 and FIG. 38).

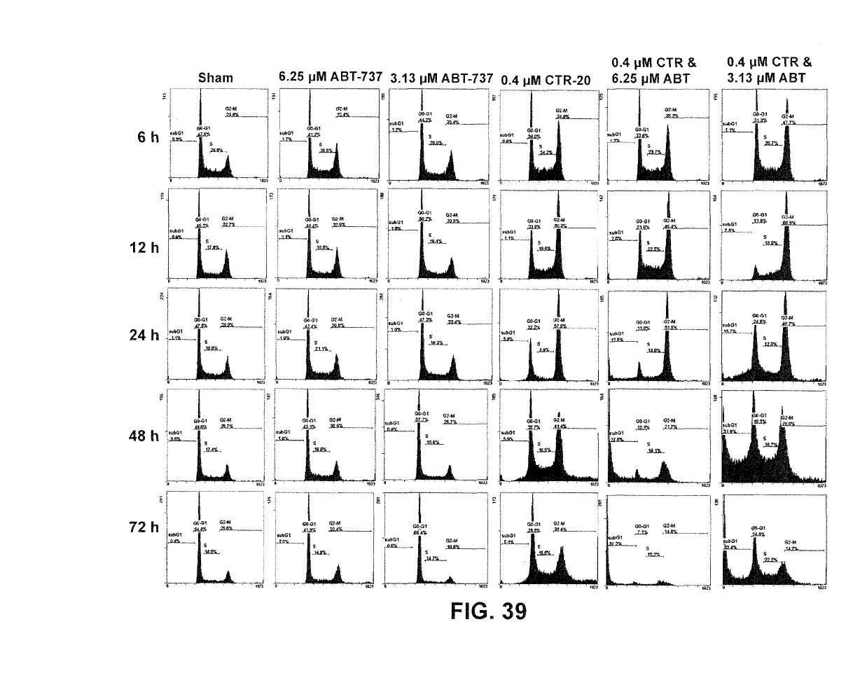

Data in FIG. 39 shows that the combination of 0.4 μM CTR-20 (CTR) and 6.25 μM ABT-737 (ABT) completely kill off the MDA-MB231 population by 72 hours post-treatment. As a single regimen, ABT-737 up to 6.25 μM did not alter cell cycle progression in any substantial way. However, MDA-MB231 cells were “permanently” arrested at G2/M in the presence of 0.4 μM CTR-20 plus 6.25 μM ABT-737, leading to apoptotic cell death (manifested by the presence of sub-G1 DNA contend) by 72 hours after the combinational treatment.

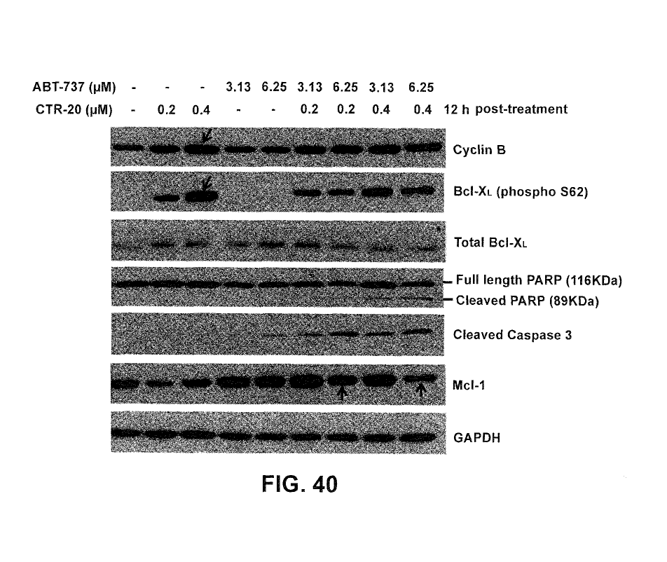

The combination of CTR-20 and ABT-737 induces apoptosis through cell cycle arrest at M and suppressing anti-apoptotic Bcl-X_{L} and Mcl-1. Data in FIG. 40 shows: (1) that the levels of cyclin B and Bcl-X_{L} phosphorylation on the serine 62 residue substantially increases in the presence of 0.4 μM of CTR-20, indicating that the cell cycle arrested at G2/M and anti-apoptotic pathway was suppressed (black arrows); (2) that the combination of 0.2-0.4 μM CTR-20 and 6.25 μM ABT-737 further suppressed the anti-apoptotic pathway by downregulating Mcl-1 (white arrows); and (3) the combination of CTR-20 (0.2-0.4 μM) and ABT-737 (3.13-6.25 μM) effectively induced apoptosis as manifested by the cleavage of PARP and caspase 3.

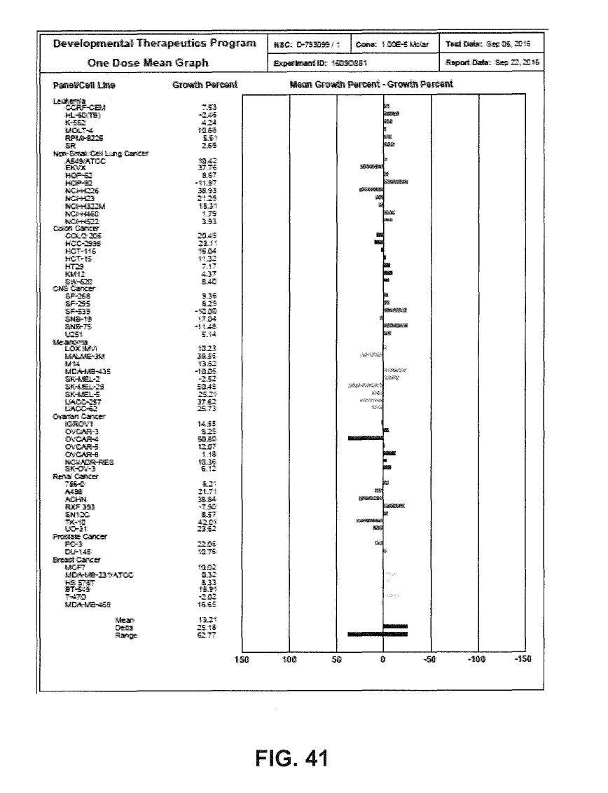

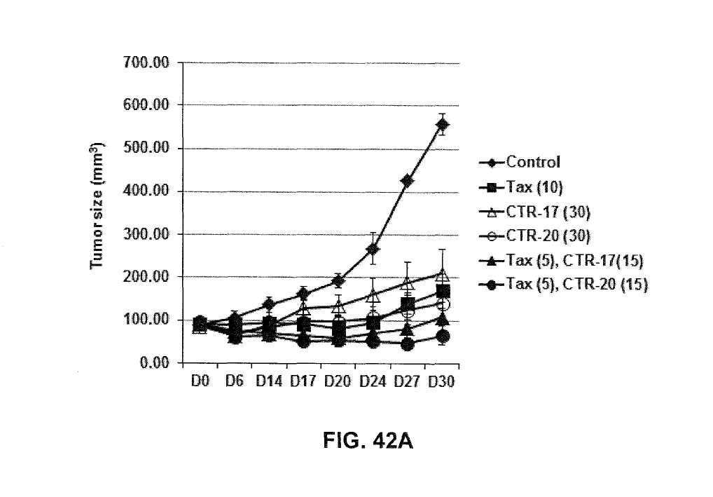

Data in FIG. 41 shows that CTR-20 effectively killed or inhibited cell proliferation of all the cell lines included in the NCI 60 cancer panel. Data from an SRB-mediated cell survival assay carried out by the US national Cancer Institute show that cells treated with 10 μM CTR-20 for 48 hours effectively killed/inhibited proliferation of all the 60 cancer cell lines including in the NCI-60 panel: six leukemia cell lines (ranging from −2.46 to +10.88%), nine non-small cell lung cancer cell lines (−11.97 to 38.93%), seven colorectal cancer cell lines (+4.37 to +23.11), six CNS cancer (−+11.48 to +17.04%), nine melanomas (−10.05 to +50.45%), seven ovarian cancer cell lines (+1.18 to +50.80%), seven renal cancer cell lines (−7.90 to +42.01%), two prostate cancer cell lines (+10.76 to +22.06), and six breast cancer cell lines (−2.02 to +18.91%).

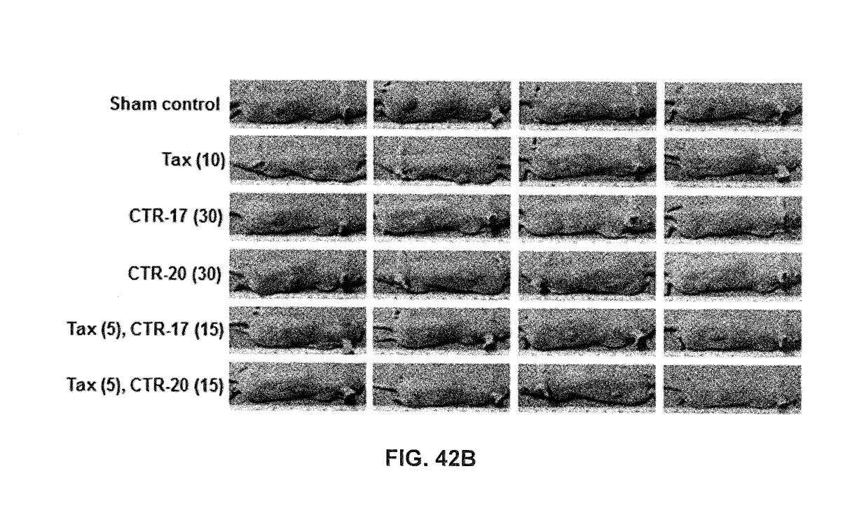

As can be seen from FIG. 42, both CTR-17 and CTR-20 showed effective anti-tumor activity in a xenograft model. FIG. 42A shows changes in tumor size (volume in mm³) in response to drug treatments, alone or in combination with paclitaxel. Experimental protocol and data are shown in Tables 11 and 12, respectively. The antitumor activities of CTR-18 and CTR-19 are shown in Table 13. Values are means±S.E.M. “D” denotes day(s) post-treatment. FIG. 42B shows exemplary images of representative ATH490 athymic mice engrafted with MDA-MB-231 human metastatic breast cancer cells that were vehicle only or treated with drugs as indicated. Numbers in brackets are mg/kg body weight. As can be seen from FIG. 42 and the Tables 11 and 12, both CTR-17 and CTR-20 showed strong antitumor activity against MDA-MB-231 metastatic breast cancer in the mouse xenograft model. The combination of a ½ dose of CTR-17 (or CTR-20) with a ½ dose of paclitaxel was considerably more effective than full dose of CTR-17, CTR-20, or paclitaxel alone. Although all four CTR compounds (CTR-17, -18, -19 and -20) showed antitumor activities, CTR-20 shows the greatest antitumor activity with mice engrafted with MDA-MB-231 metastatic breast cancer cells. This result is consistent with that of the in vitro study.

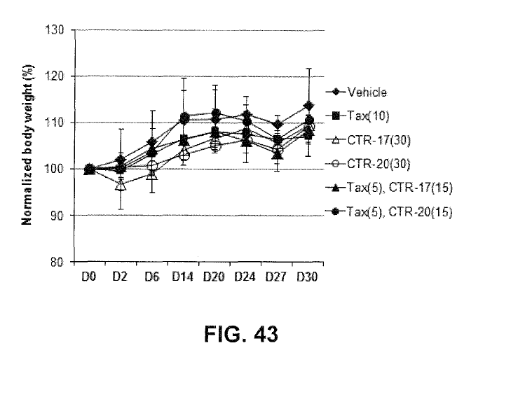

Data from body weight analysis indicates that CTR compounds are not toxic to mice (FIG. 43). Six-week old ATH40 athymic nude mice were treated as indicated in the legend. “Tax” denotes that paclitaxel was injected by i.v. as described in Table 11. D0-D30 denotes day 0 to day 30 post-drug treatment. The numbers in brackets show drug concentrations in mg/kg body weight. The body weights of ATH490 mice were normalized based on the total body weight on day 0 (100%). Neither CTR-17 nor CTR-20 caused any notable toxic side effect to ATH490 athymic mice, as determined by the changes in body weights.

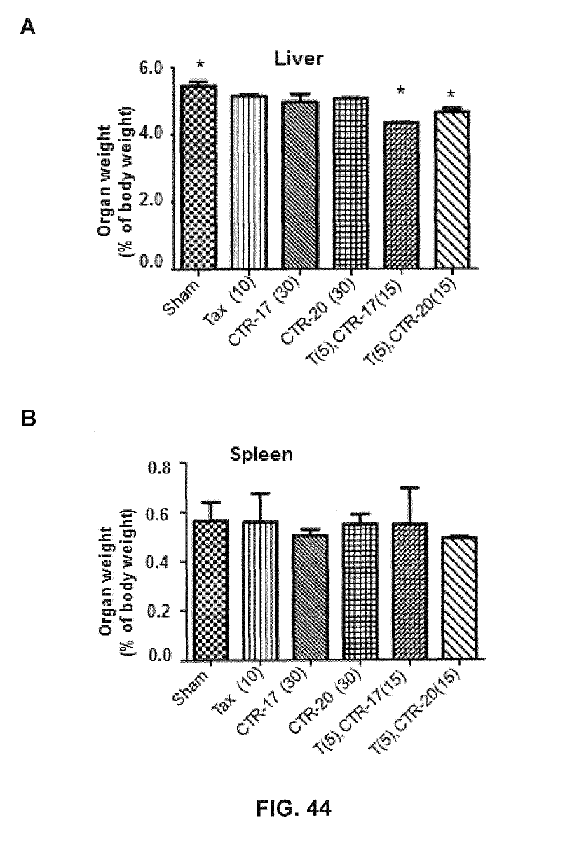

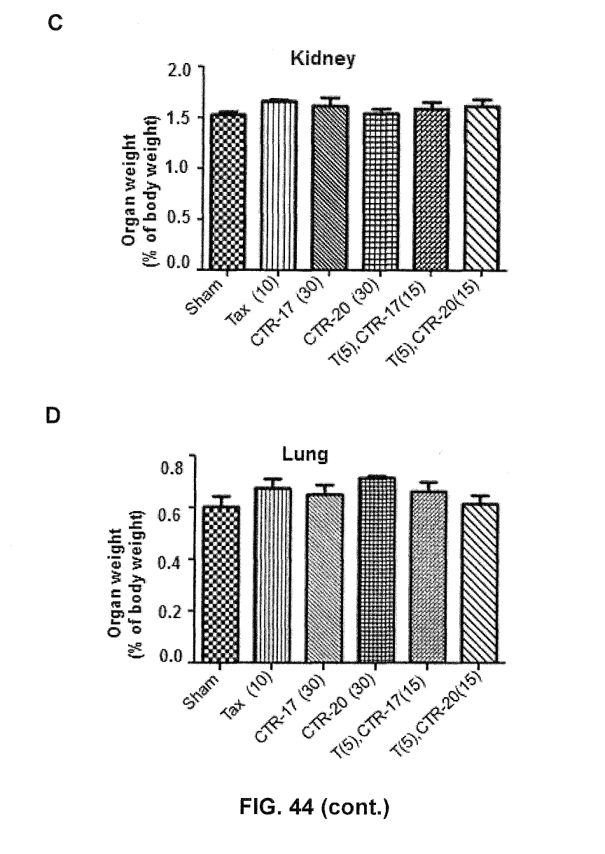

Neither CTR-17 nor CTR-20 was observed to cause any notable ill-effects to mouse vital organs. The weights of four different organs (liver, spleen, kidney and lung) of ATH490 mice from different treatment groups (as described in Table 11) were measured at 30-day post-treatment. Analysis was performed using GraphPad Prism software (GraphPad Software). All values are presented as mean±S.E.M. Comparison between each group was made by p values determined using one-way ANOVA. A p value of <0.05 is considered to be statistically significant. The data shows that there was no significant difference in the mass of spleen, kidney, and lung between vehicle only and drug-treated groups (p values for spleen, kidney and lung were 0.99, 0.74, and 0.36, respectively). However, the liver sizes of the samples treated in combination with paclitaxel and CTR compounds were somewhat smaller than those of the vehicle only control. (p=0.0003). Each organ weight (%) was normalized with total body weight (BW). This data suggests, while not wishing to be limited by theory, that the treatment of ATH490 mice with 30 mg/kg of CTR-17 or 30 mg/kg of CTR-20 does not cause any notable ill-effects on the animals, as determined by changes in the weights of the four vital organs (liver, spleen, kidney and lung) shown in FIG. 44.

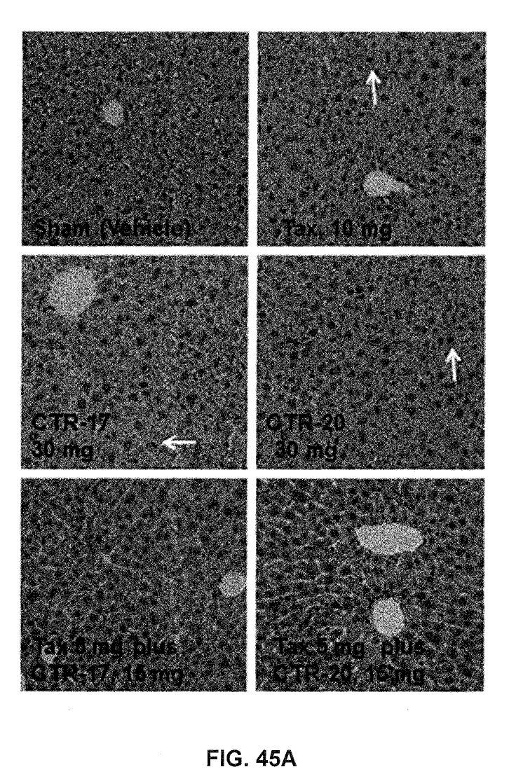

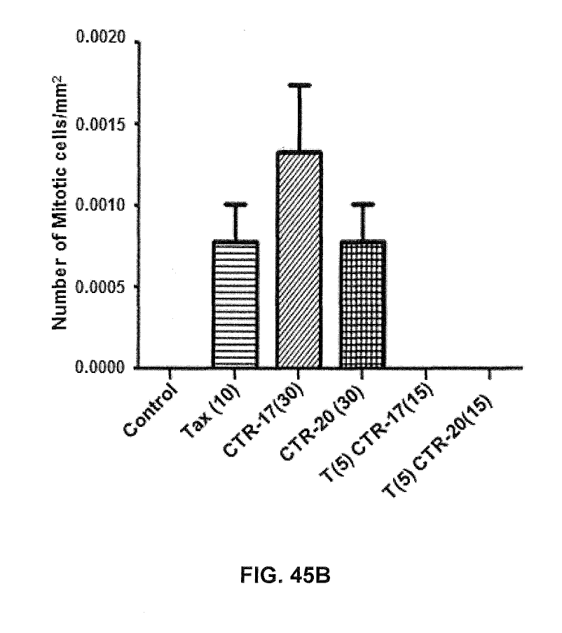

FIG. 45 shows exemplary images of the effects of CTR-17 and CTR-20 on the liver. ATH490 athymic mice were treated as indicated in the listed concentrations for 30 days (FIG. 45A), and then the liver cell proliferation analyzed by examining the number of mitotic cells (white arrows in FIG. 45A). “Tax” denotes paclitaxel. The numbers in the brackets in (FIG. 45B) are mg/kg body weight. As can be seen from FIG. 45 and Table 14, livers of animals treated with 10 mg paclitaxel, 30 mg CTR-17, or 30 mg CTR-20 showed small increases in the mitotic index. However, this small increase is considered normal as the AST/ALT ratio is <3 (Table 14). The small increase in mitotic cells in the liver tissue was completely prevented when ½ dose of paclitaxel (5 mg) and ½ dose of either CTR-17 (15 mg) or CTR-20 (15 mg) were used in combination (p<0.0001).

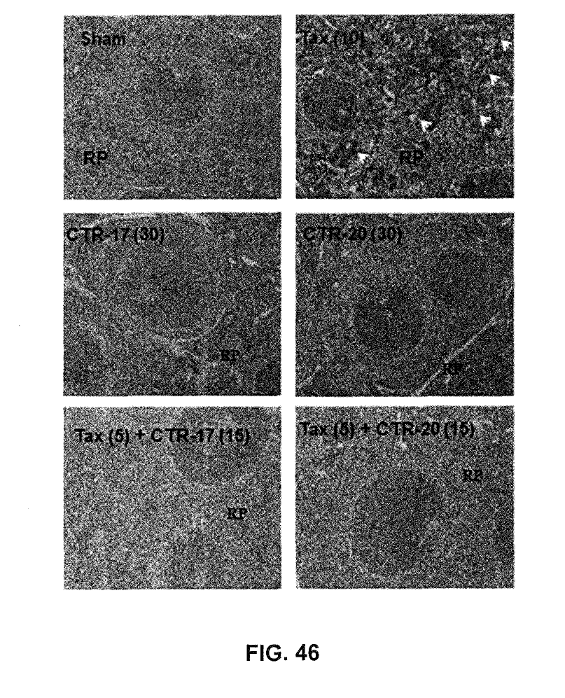

Neither CTR-17 nor CTR-20 was observed to cause any notable toxicity to spleen. ATH490 athymic mice were sham-treated (vehicle only) or treated with compounds of the indicated doses for 30 days, followed by toxicity analysis after spleen tissues were H & E stained. The numbers in the brackets are mg per kg of body weight. Arrows indicate the presence of macrophages in the red pulp (RP). Images were taken using a Zeiss EPI-fluorescent microscope (10× objective). The toxicity on the spleen is summarized in Table 15. Drug administration was carried out as described in Table 11. As can be seen from FIG. 46 and Table 15, unlike animals treated with paclitaxel (10 mg/kg), which showed considerable side effects in the spleen including an increase in cellularity, hyperplasia of myeloid and lymphoid cells, those treated with 30 mg/kg CTR-17 or 30 mg/kg CTR-20 did not show any notable ill-effects to spleen tissues, except a minor increase in myeloid elements in the red pulp. Animals that were treated with a ½ dose of CTR-20 (15 mg) and a ½ dose of paclitaxel (5 mg) did not show any ill-effects to spleen.

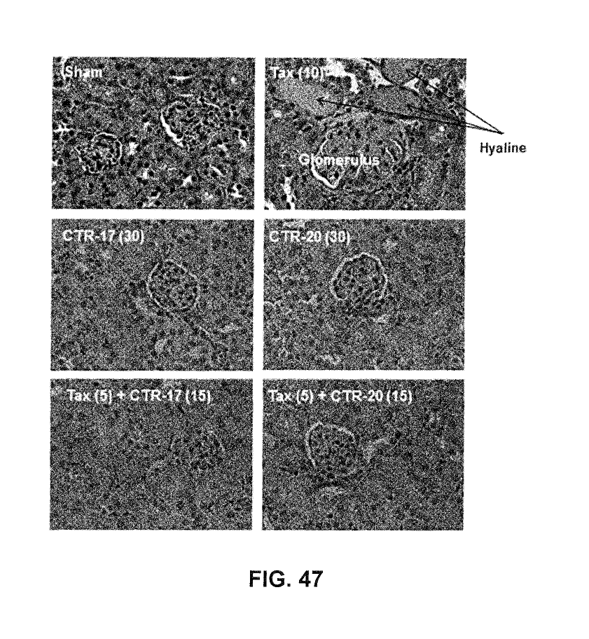

FIG. 47 shows exemplary images of the effects of CTR-17 and CTR-20 on the kidney. ATH490 mice were treated as described in Table 11. At day 30, kidneys were harvested, stained with H&E, and observed under a Zeiss EPI-fluorescent microscope (40× objective). Treatment of animals with CTR-17 (30 mg/kg), CTR-20 (30 mg/kg) or paclitaxel (5 mg/kg) combined with either CTR-17 (15 mg/kg) or CTR-20 (15 mg/kg) generally did not cause any notable ill-effects to the kidney. However, when mice were treated with paclitaxel (10 mg/kg), approximately 1 out of 5 mice showed renal abnormalities with the appearance of glassy and acellular hyalines and hypo-cellularity with expanded space of glomeruli.

### V. Discussion

With a central core composed of an aromatic ketone and an enone group, chalcone-based compounds (Scheme 3) have been reported to show potent anti-tubulin activity (Lu et al., 2012).

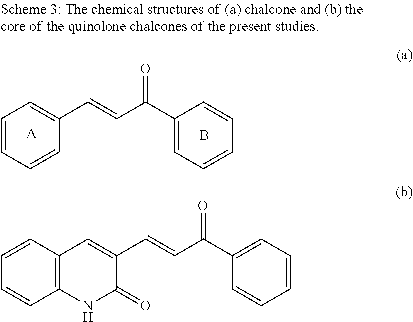

The binding of chalcones to tubulin was reported to be inhibited by colchicine and podophyllotoxins, suggesting that certain chalcone-based compounds may effectively bind to β-tubulin through the colchicine binding site or very close to it (Ducki et al., 2005; Ducki et al., 2009; Hadfield et al., 2003; Lawrence et al., 2000; Peyrot et al., 1992). Other studies also showed that chalcone-based compounds bind to tubulin reversibly and rapidly, thus inhibiting the microtubule assembly (Stanton et al., 2011).

The development of effective and safe anticancer agents targeting microtubules based on a chalcone scaffold is of medicinal interest.

Ten quinolone chalcones with a range of substituents such as a nitro group, methoxy and methyl groups, and halogen atoms (F, Cl and Br) in the aryl ring A were synthesized and then screened for anticancer activity against three breast cancer cell lines and one or two non-cancer breast cell lines. The results indicated that among the different substitutions tried, 2-methoxy substitution in the aryl ring A enhanced both the selectivity and growth inhibitory potency against breast cancer cells with IC₅₀ values of 0.41, 0.15 and 0.52 μM against MDA-MB-231, MDA-MB-468, and MCF-7 cells, respectively. Hence, then 24 novel quinolone chalcones were synthesized that had a 2-alkoxy substitution in phenyl ring A (Table 1) and the anticancer activities of some of these compounds were then examined (Tables 2-4).

The compounds CTR-17 and CTR-20, showed preferential killing of cancer over non-cancer cells, up to 24-26 fold. Furthermore, the IC₅₀ values in killing a variety of different cancer cell lines by CTR-17 and CTR-20 were found to be in the sub-μM range, making both of them promising leads. Further studies showed that all of the 24 novel quinolone chalcone compounds effectively kill cancer cells, many of which preferentially kill cancer over non-cancer cells. A study with isogenic cell lines showed that CTR-20, CTR-21 and CTR-32 preferentially kill the fully malignant MCF10CA1a breast cancer cells over premalignant MCF10AT1 and non-cancer MCF10A breast cells. Previously, we identified that both CTR-17 and CTR-20 killed two different lines of multidrug-resistant cells (overexpressing MDR1 or MRP) almost as effectively as non-resistant cells (or better in some cases). In contrast, colchicine, paclitaxel and vinblastine are at least 10-fold less effective in killing multidrug-resistant cells than non-resistant control cells. It was found that CTR-21 and CTR-32 kill the multidrug- and paclitaxel-resistant MDA-MB231TaxR breast cancer cells with high potency. CTR-20 also effectively kills two bortezomib-resistant multiple myeloma cell lines (RPMI-8226-BR and ANBL6-BR), and this data indicates that CTR compounds effectively overcome the drug-resistant issue which is currently a major cause of chemotherapy failure.

The data showed that both CTR-17 and CTR-20 bind to tubulin, resulting in the inhibition of microtubule polymerization. We have also shown that the tubulin binding sites of CTR-17 and CTR-20 closely overlap with that of colchicine, but apart from the vinblastine binding site. Data from in silico molecular modeling suggests that CTR-17, CTR-20 and colchicine, respectively, form one, two and three H-bonds with amino acid residues on tubulin, in addition to strong Van der Waals interactions.

It is well known that agents disrupting microtubule dynamics through the binding to the colchicine-binding site have minimal drug-resistant issues, although they tend to be quite toxic to humans. The finding that compounds such as CTR-20, CTR-21 and CTR-32 can overcome drug resistance is consistent with this previous finding as at least CTR-17 and CTR-20 bind to the colchicine-binding site.

In contrast to colchicine, which is known to be very toxic to humans (Lu et al. 2012), compounds such as CTR-17 and CTR-20 show little toxicity to animals (FIGS. 43-47), which is consistent with the in vitro data (Table 2). Compounds such as CTR-21 and CTR-32, similarly to CTR-20, kill cells in a malignancy-dependent manner. This notion is strengthened as the inhibition of microtubule dynamics by CTR-17, CTR-20, CTR-21 and CTR-32 is reversible upon washing off the compounds (manifested by recovering cell cycle progression).

While not wishing to be limited by theory, the number of H-bonds between compounds (for example, CTR-17, CTR-20 and colchicine) and amino acid residues of tubulin can be relevant to differences in efficacy, reversibility and toxicity. In this respect, CTR-20, which shows two H-bonds, is quite effective on many different cancers (Tables 2 and 3) while still reversible (FIG. 17). Furthermore, CTR-20 shows preferential cancer cell killing over non-cancer cells (Table 2). CTR-17 and CTR-20 were not observed to cause DNA damage nor impede DNA replication.

Data from the experiments with mice engrafted with the MDA-MB-231 metastatic breast cancer show that the efficacy of CTR-20 is almost comparable with that of paclitaxel (although CTR-20 and paclitaxel were used at a dose of 30 mg/kg and 10 mg/kg, respectively, the former was given by i.p. and the latter was given by i.v.). However, CTR-20 is less toxic (FIG. 46). Further, the combination of ½ doses of CTR-20 and paclitaxel was much more effective than a full dose of either CTR-20 or paclitaxel alone.

Together, the in vitro data shows that novel tubulin-targeting compounds, such as CTR-17, CTR-20, CTR-21 and CTR-32 preferentially kill many different cancer cells including all of the cell lines contained in the NCI-60 cancer panel and the MDR1- and MRP1-overexpressing multidrug-resistant cancer cells (which are also resistant to paclitaxel, vinblastine and colchicine). Data from mice engrafted with metastatic breast tumor cells showed that both of these CTR compounds possess strong antitumor activities, when used alone or in combination with paclitaxel.

While the present disclosure has been described with reference to what are presently considered to be the preferred examples, it is to be understood that the present disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### FULL CITATIONS FOR DOCUMENTS REFERRED TO IN THE SPECIFICATION

Borisy, G. G. and Taylor, E. W. (1967a). The mechanism of action of colchicine. Binding of colchincine-3H to cellular protein. J. Cell Biol. 34, 525-533.
Borisy, G. G. and Taylor, E. W. (1967b). The mechanism of action of colchicine. Colchicine binding to sea urchin eggs and the mitotic apparatus. J. Cell Biol. 34, 535-548.
Chou, T. C. (2006). Theoretical basis, experimental design, and computerized simulation of synergism and antagonism in drug combination studies. Pharmacol. Rev. 58, 621-681.
Dominguez, J. N., Charris, J. E., Lobo, G., Gamboa de, D. N., Moreno, M. M., Riggione, F., Sanchez, E., Olson, J., and Rosenthal, P. J. (2001). Synthesis of quinolinyl chalcones and evaluation of their antimalarial activity. Eur. J. Med. Chem. 36, 555-560.
Ducki, S., Mackenzie, G., Greedy, B., Armitage, S., Chabert, J. F., Bennett, E., Nettles, J., Snyder, J. P., and Lawrence, N. J. (2009). Combretastatin-like chalcones as inhibitors of microtubule polymerisation. Part 2: Structure-based discovery of alpha-aryl chalcones. Bioorg. Med. Chem. 17, 7711-7722.
Ducki, S., Mackenzie, G., Lawrence, N. J., and Snyder, J. P. (2005). Quantitative structure-activity relationship (5D-QSAR) study of combretastatin-like analogues as inhibitors of tubulin assembly. J. Med. Chem. 48, 457-465.
Dumontet, C. and Jordan, M. A. (2010). Microtubule-binding agents: a dynamic field of cancer therapeutics. Nat. Rev. Drug Discov. 9, 790-803.
Gigant, B., Wang, C., Ravelli, R. B., Roussi, F., Steinmetz, M. O., Curmi, P. A., Sobel, A., and Knossow, M. (2005). Structural basis for the regulation of tubulin by vinblastine. Nature 435, 519-522.
Hadfield, J. A., Ducki, S., Hirst, N., and McGown, A. T. (2003). Tubulin and microtubules as targets for anticancer drugs. Prog. Cell Cycle Res. 5, 309-325.
Hu, C., Solomon, V. R., Ulibarri, G., and Lee, H. (2008). The efficacy and selectivity of tumor cell killing by Akt inhibitors are substantially increased by chloroquine. Bioorg. Med. Chem. 16, 7888-7893.
Huang, G. S., Lopez-Barcons, L., Freeze, B. S., Smith, A. B., III, Goldberg, G. L., Horwitz, S. B., and McDaid, H. M. (2006). Potentiation of taxol efficacy and by discodermolide in ovarian carcinoma xenograft-bearing mice. Clin. Cancer Res. 12, 298-304.
Kuppens, I. E. (2006). Current state of the art of new tubulin inhibitors in the clinic. Curr. Clin. Pharmacol. 1, 57-70.
Lawrence, N. J., McGown, A. T., Ducki, S., and Hadfield, J. A. (2000). The interaction of chalcones with tubulin. Anticancer Drug Des 15, 135-141.
Li, R., Kenyon, G. L., Cohen, F. E., Chen, X., Gong, B., Dominguez, J. N., Davidson, E., Kurzban, G., Miller, R. E., Nuzum, E. O., Rosenthal, P. J. and McKerrow, J. H. (1995). In vitro antimalarial activity of chalcones and their derivatives. J. Med. Chem. 38, 5031-5037.
Lu, Y., Chen, J., Xiao, M., Li, W., and Miller, D. D. (2012). An overview of tubulin inhibitors that interact with the colchicine binding site. Pharm. Res. 29, 2943-2971.
Meth-Cohn, O., Narine, B., and Tarnowski, B. (1981). A versatile new synthesis of quinolines and related fused pyridines, Part 5. The synthesis of 2-quinoline-3-carbaldehydes. Journal of the Chemical Society, Perkin Transactions 1, 1520-1530.
Peyrot, V., Leynadier, D., Sarrazin, M., Briand, C., Menendez, M., Laynez, J., and Andreu, J. M. (1992). Mechanism of binding of the new antimitotic drug MDL 27048 to the colchicine site of tubulin: equilibrium studies. Biochemistry 31, 11125-11132.
Pryor, D. E., O'Brate, A., Bilcer, G., Diaz, J. F., Wang, Y., Wang, Y., Kabaki, M., Jung, M. K., Andreu, J. M., Ghosh, A. K., Giannakakou, P., and Hamel, E. (2002). The microtubule stabilizing agent laulimalide does not bind in the taxoid site, kills cells resistant to paclitaxel and epothilones, and may not require its epoxide moiety for activity. Biochemistry 41, 9109-9115.
Ravelli, R. B., Gigant, B., Curmi, P. A., Jourdain, I., Lachkar, S., Sobel, A., and Knossow, M. (2004). Insight into tubulin regulation from a complex with colchicine and a stathmin-like domain. Nature 428, 198-202.
Singh, P., Rathinasamy, K., Mohan, R., and Panda, D. (2008). Microtubule assembly dynamics: an attractive target for anticancer drugs. IUBMB. Life 60, 368-375.
Skehan, P., Storeng, R., Scudiero, D., Monks, A., McMahon, J., Vistica, D., Warren, J. T., Bokesch, H., Kenney, S., and Boyd, M. R. (1990). New colorimetric cytotoxicity assay for anticancer-drug screening. J. Natl. Cancer Inst. 82, 1107-1112.
Srivastava, A. and Singh, R. (2005). Vilsmeier-Haack reagent: a facile synthesis of 2-chloro-3-formylquinolines from N-arylacetamides and transformation into different functionalities. Indian Journal of Chemistry Section B 44, 1868.
Stanton, R. A., Gernert, K. M., Nettles, J. H., and Aneja, R. (2011). Drugs that target dynamic microtubules: a new molecular perspective. Med. Res. Rev. 31, 443-481.
Tokesi, N., Lehotzky, A., Horvath, I., Szabo, B., Olah, J., Lau, P., and Ovadi, J. (2010). TPPP/p25 promotes tubulin acetylation by inhibiting histone deacetylase 6. J. Biol. Chem. 285, 17896-17906.
Vogel, A. I., Tatchell, A. R., Furnis, B. S., Hannaford, A. J., and Smith, P. W. G. (1996). Vogel's Textbook of Practical Organic Chemistry. Prentice Hall).
Weisenberg, R. C., Borisy, G. G., and Taylor, E. W. (1968). The colchicine-binding protein of mammalian brain and its relation to microtubules. Biochemistry 7, 4466-4479.
Zhou, J. and Giannakakou, P. (2005). Targeting microtubules for cancer chemotherapy. Curr. Med. Chem. Anticancer Agents 5, 65-71.

TABLE 1
Chemical names and structures.

| Structure | Compound Structure | Experiment |
|---|---|---|
| Number | (Chemical Name) | Book Code |
| 1 | 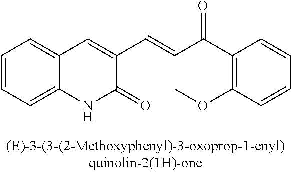 [SMILES: COc1ccccc1C(=O)/C=C/c1cc2ccccc2[nH]c1=O] | CTR-17 |
| 2 | 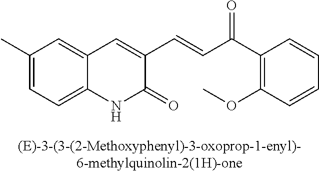 [SMILES: COc1ccccc1C(=O)/C=C/c1cc2cc(C)ccc2[nH]c1=O] | CTR-18 |
| 3 | 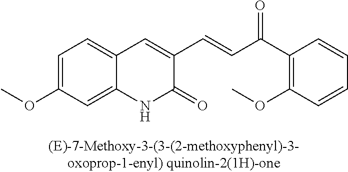 [SMILES: COc1ccc2cc(/C=C/C(=O)c3ccccc3OC)c(=O)[nH]c2c1] | CTR-19 |
| 4 | 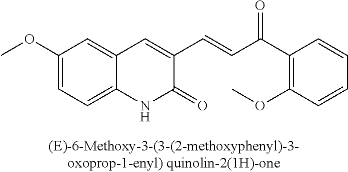 [SMILES: COc1ccc2[nH]c(=O)c(/C=C/C(=O)c3ccccc3OC)cc2c1] | CTR-20 |
| 5 | 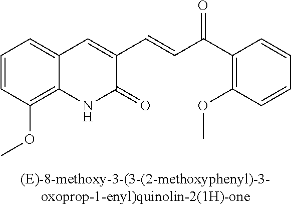 [SMILES: COc1ccccc1C(=O)/C=C/c1cc2cccc(OC)c2[nH]c1=O] | CTR-21 |
| 6 | 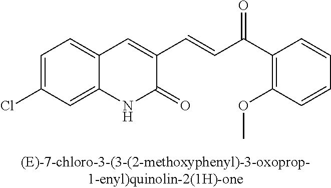 [SMILES: COc1ccccc1C(=O)/C=C/c1cc2ccc(Cl)cc2[nH]c1=O] | CTR-22 |
| 7 | 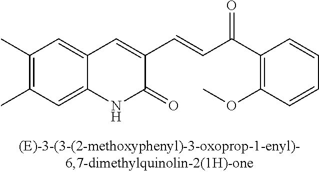 [SMILES: COc1ccccc1C(=O)/C=C/c1cc2cc(C)c(C)cc2[nH]c1=O] | CTR-23 |
| 8 | 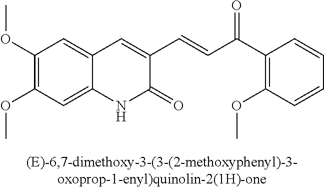 [SMILES: COc1cc2cc(/C=C/C(=O)c3ccccc3OC)c(=O)[nH]c2cc1OC] | CTR-24 |
| 9 | 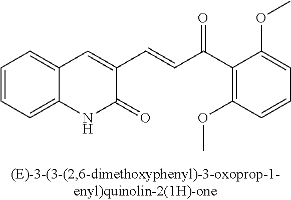 [SMILES: COc1cccc(OC)c1C(=O)/C=C/c1cc2ccccc2[nH]c1=O] | CTR-25 |
| 10 | 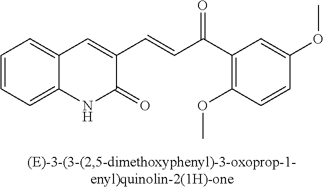 [SMILES: COc1ccc(OC)c(C(=O)/C=C/c2cc3ccccc3[nH]c2=O)c1] | CTR-26 |
| 11 | 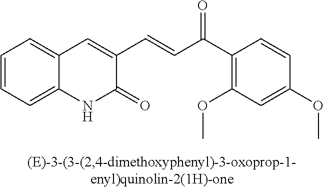 [SMILES: COc1ccc(C(=O)/C=C/c2cc3ccccc3[nH]c2=O)c(OC)c1] | CTR-27 |
| 12 | 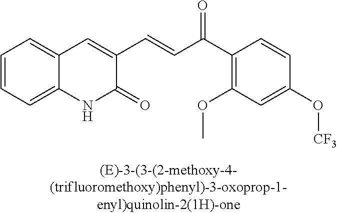 [SMILES: COc1cc(OC(F)(F)F)ccc1C(=O)/C=C/c1cc2ccccc2[nH]c1=O] | CTR-28 |
| 13 | 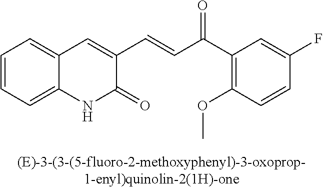 [SMILES: COc1ccc(F)cc1C(=O)/C=C/c1cc2ccccc2[nH]c1=O] | CTR-29 |
| 14 | 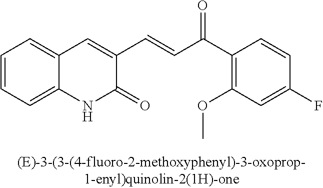 [SMILES: COc1cc(F)ccc1C(=O)/C=C/c1cc2ccccc2[nH]c1=O] | CTR-30 |
| 15 | 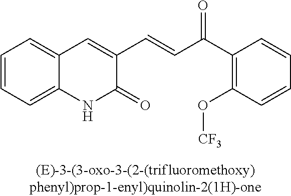 [SMILES: O=C(/C=C/c1cc2ccccc2[nH]c1=O)c1ccccc1OC(F)(F)F] | CTR-31 |
| 16 | 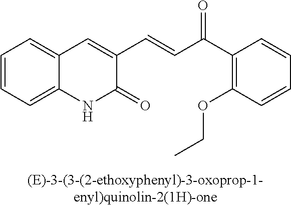 [SMILES: CCOc1ccccc1C(=O)/C=C/c1cc2ccccc2[nH]c1=O] | CTR-32 |
| 17 | 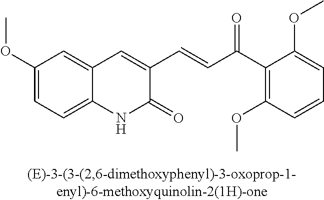 [SMILES: COc1ccc2[nH]c(=O)c(/C=C/C(=O)c3c(OC)cccc3OC)cc2c1] | CTR-33 |
| 18 | 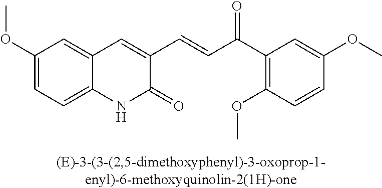 [SMILES: COc1ccc(OC)c(C(=O)/C=C/c2cc3cc(OC)ccc3[nH]c2=O)c1] | CTR-34 |
| 19 | 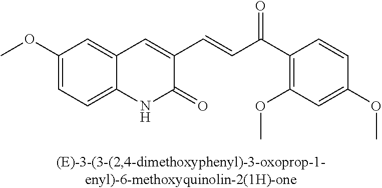 | CTR-35 |
| 20 | 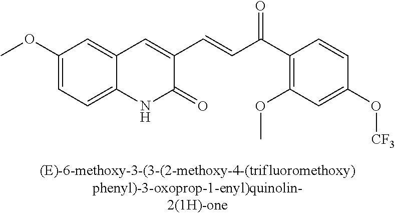 [SMILES: COc1ccc2[nH]c(=O)c(/C=C/C(=O)c3ccc(OC(F)(F)F)cc3OC)cc2c1] | CTR-36 |
| 21 | 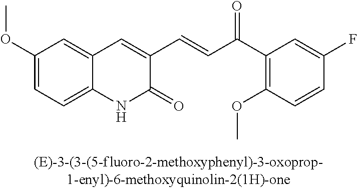 [SMILES: COc1ccc2[nH]c(=O)c(/C=C/C(=O)c3cc(F)ccc3OC)cc2c1] | CTR-37 |
| 22 | 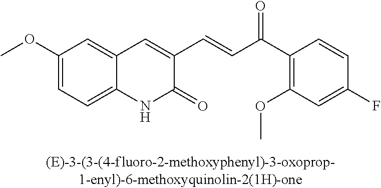 [SMILES: COc1ccc2[nH]c(=O)c(/C=C/C(=O)c3ccc(F)cc3OC)cc2c1] | CTR-38 |
| 23 | 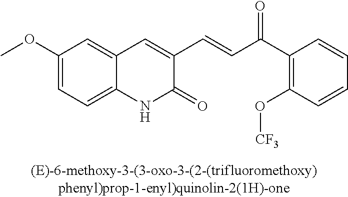 [SMILES: COc1ccc2[nH]c(=O)c(/C=C/C(=O)c3ccccc3OC(F)(F)F)cc2c1] | CTR-39 |
| 24 | 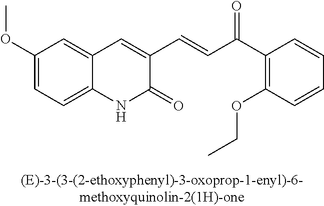 [SMILES: CCOc1ccccc1C(=O)/C=C/c1cc2cc(OC)ccc2[nH]c1=O] | CTR-40 |

TABLE 2
Initial screening of four CTR compounds using breast cancer cells (MDA-MB-231, MDA-
MB-468, MCF-7) and non-cancer breast cells (184B5) determined by SRB assays.

| | IC₅₀ (μM) ^{a,b} | | | | | | |
|---|---|---|---|---|---|---|---|
| CODE | MDA-MB-231 | MDA-MB-468 | MCF-7 | K562 | HeLa | 184B5^{d} | MCF-10A^{d} |
| CTR-17^{c} | 0.41 ± 0.02 | 0.15 ± 0.33 | 0.52 ± 0.17 | 0.15 ± 0.10 | 0.33 ± 0.06 | 3.49 ± 0.03 | 3.95 ± 0.14 |
| CTR-18 | 0.93 | 0.08 | 0.12 | 0.21 ± 0.02 | ND | 3.89 | ND^{e} |
| CTR-19 | 5.11 | 1.91 | 1.93 | 0.95 ± 0.65 | ND | 3.94 | ND |
| CTR-20^{c} | 0.12 ± 0.09 | 0.12 ± 0.02 | 0.14 ± 0.06 | 0.13 ± 0.02 | 0.10 ± 0.02 | 1.24 ± 0.06 | 2.37 ± 0.21 |
| Chloroquine | 22.52 | 28.58 | 38.44 | ND | ND | 76.13 | ND |
| Cisplatin | 23.65 | 31.02 | 25.77 | ND | ND | 25.54 | ND |

^{a} IC₅₀ values were calculated from Sigmoidal dose response curves (variable slope), which were generated with GraphPad Prism V. 4.02 (GraphPad Software Inc.).
^{b} Values are the mean value of triplicates of at least two independent experiments.
^{c}CTR-17 and CTR-20 were repeated twice.
^{d}184B5 and MCF-10A are non-cancer, immortalized breast epithelial cell lines, and the rest are different cancer cell lines.
^{e}ND, not determined.

TABLE 3
Antiproliferation effects of CTR-17 and CTR-20 on other cancer cell lines.

| | U87MG | T98G | NCI-H1975 | A549 | RPMI-8226 | UC3 | HEK293T |
|---|---|---|---|---|---|---|---|
| | (brain) | (brain) | (lung) | (lung) | (myeloma) | (Bladder) | (kidney) |
| CTR-17^{b} | 0.76 ± 0.10^{a} | 0.82 ± 0.09 | 0.60 ± 0.14 | 0.41 ± 0.06 | 0.36 ± 0.04 | 0.39 ± 0.03 | 0.42 ± 0.07 |
| CTR-20^{b} | 0.49 ± 0.13 | 0.22 ± 0.10 | 0.39 ± 0.14 | 0.13 ± 0.04 | 0.23 ± 0.00 | 0.12 ± 0.03 | 0.19 ± 0.00 |

^{a}Numbers are IC₅₀ values in μM, determined by SRB assays as described in Table 2.
^{b}Treatment with CTR-17 and CTR-20 was for 72 hours.

TABLE 4
Antiproliferation effects of CTR compounds on cancer and non-cancer cells as determined by SRB assays

| | IC₅₀ ^{a, b} | | | | | |
|---|---|---|---|---|---|---|
| CODE | MDA-MB231 | MCF7 | HeLa | RPMI-8226 | 184B5^{d} | MCF10A^{d} |
| CTR-21 (nM) | 56.91 ± 10.46 | 75.11 ± 3.07 | 11.93 ± 1.40 | 5.34 ± 0.89 | 36.96 ± 2.5 | 20.32 ± 2.47 |
| CTR-23 (μM) | 1.52 ± 0.12 | 0.50 ± 0.10 | 0.64 ± 0.05 | 1.41 ± 0.15 | 1.32 ± 0.03 | 1.12 ± 0.04 |
| CTR-24 (μM) | 2.02 ± 0.01 | 1.54 ± 0.36 | 1.34 ± 0.20 | 1.04 ± 0.13 | 2.37 ± 0.19 | 2.10 ± 0.39 |
| CTR-25 (μM) | 2.60 ± 0.18 | 1.69 ± 0.07 | 1.32 ± 0.02 | 0.77 ± 0.21 | 2.64 ± 0.17 | 2.64 ± 0.15 |
| CTR-26 (μM) | 0.96 ± 0.15 | 0.32 ± 0.01 | 0.29 ± 0.02 | 0.20 ± 0.03 | 0.77 ± 0.11 | 0.91 ± 0.03 |
| CTR-27 (μM) | 2.69 ± 0.24 | 2.20 ± 0.35 | 1.80 ± 0.27 | 0.77 ± 0.10 | 2.80 ± 0.18 | 2.52 ± .03 |
| CTR-29 (μM) | 0.21 ± 0.02 | 0.10 ± 0.01 | 0.09 ± 0.02 | 0.07 ± 0.01 | 0.28 ± 0.01 | 0.27 ± 0.05 |
| CTR-30 (μM) | 1.42 ± 0.08 | 1.47 ± 0.15 | 1.50 ± 0.13 | 0.20 ± 0.03 | 2.46 ± 0.14 | 2.79 ± 0.59 |
| CTR-32 (nM) | 44.22 ± 4.21 | 46.36 ± 3.61 | 12.88 ± 0.35 | 6.29 ± 1.43 | 52.03 ± 7.35 | 30.06 ± 1.61 |
| CTR-33 (μM) | 1.36 ± 0.11 | 1.71 ± 0.25 | 0.10 ± 0.20 | 1.17 ± 0.11 | 4.68 ± 0.52 | 2.60 ± 0.73 |
| CTR-34 (μM) | 0.84 ± 0.08 | 0.85 ± 0.06 | 0.64 ± 0.03 | 0.26 ± 0.02 | 1.16 ± 0.10 | 0.98 ± 0.04 |
| CTR-35 (μM) | 2.35 ± 0.26 | 2.12 ± 0.36 | 2.07 ± 0.25 | 1.32 ± 0.10 | 4.29 ± 0.06 | 2.88 ± 0.54 |
| CTR-36 (μM) | 0.73 ± 0.06 | 1.23 ± 0.08 | 1.11 ± 0.13 | 0.15 ± 0.02 | 1.84 ± 0.07 | 1.35 ± 0.16 |
| CTR-37 (μM) | 0.39 ± 0.09 | 0.32 ± 0.04 | 0.14 ± 0.03 | 0.10 ± 0.03 | 0.44 ± 0.02 | 0.46 ± 0.11 |
| CTR-38 (μM) | 0.15 ± 0.01 | 0.14 ± 0.02 | 0.06 ± 0.01 | 0.04 ± 0.01 | 0.16 ± 0.02 | 0.11 ± 0.00 |
| CTR-40 (μM) | 0.13 ± 0.02 | 0.09 ± 0.01 | 0.04 ± 0.00 | 0.05 ± 0.02 | 0.10 ± 0.01 | 0.07 ± 0.00 |

^{a} IC₅₀ values were calculated from Sigmoidal dose response curves (variable slope), which were generated with GraphPad Prism V. 4.02 (GraphPad Software Inc.).
^{b} Values are the mean value of triplicates of at least two independent experiments.
^{d}The 184B5 and MCF10A are non-cancer, immortalized breast epithelial cell lines, and the rest is cancer cell lines.
*Cells were treated for 72 hours by the CTR compounds.
* All cell lines were authenticated on April 10, Jul. 13, 2015 & Sep. 9, 2016 (Genetica DNA Laboratories) by the STR profiling of gDNA (www.celllineauthentication.com).

TABLE 5
The number of cells with a monoplar centrosome
increases in response to CTR-17 (3 μM).

| | 12 h post-treatment | | 24 h post-treatment | |
|---|---|---|---|---|
| | Monopolar^{a} | ACS^{b} | Monopolar | ACS |
| HeLa | 80.0 ± 4.5 | 20.0 ± 4.5 | 100.0 ± 0.0 | 0.0 ± 0.0 |
| MDA-MB-231 | 40.0 ± 10.2 | 60.0 ± 10.2 | 44.9 ± 5.7 | 55.1 ± 5.7 |
| MDA-MB-468 | 55.3 ± 8.9 | 44.7 ± 8.9 | 76.0 ± 5.9 | 24.0 ± 5.9 |
| HEK293T | 50.5 ± 10.1 | 48.2 ± 10.1 | 93.2 ± 3.4 | 0.1 ± 0.0 |

^{a,b}The numbers in monopolar centrosome and abnormal chromosome segregation (ACS) are percent of total mitotic cells.

TABLE 6
The number of cells with a monoplar centrosome
increases in response to 1 μM CTR-20.

| | Monopolar (%) | ACA/S^{a} (%) |
|---|---|---|
| MB231 | 35.73 ± 5.59 | 58.05 ± 6.21 |
| HeLa | 48.13 ± 3.34 | 34.97 ± 1.96 |
| MCF-7 | 62.41 ± 2.99 | 29.01 ± 2.50 |

^{a}ACA/S: Abnormal chromosome alignment and segregation.

TABLE 7
CTR-17 and CTR-20 effectively kill multidrug-resistant
cells (KB-C-2 & H69AR). Numbers are IC₅₀ in nM or μM.

| | KB-C-1 | KB-C-2 | Resistance (fold) | SW-1271 | H69AR | Resistance (fold) |
|---|---|---|---|---|---|---|
| Colchicine (nM) | 5.36 ± 0.54 | 83.45 ± 7.22 | 15.57 | 4.84 ± 0.80 | 22.97 ± 3.63 | 4.74 |
| Paclitaxel (nM) | 2.01 ± 0.17 | 23.08 ± 0.21 | 11.48 | 4.51 ± 0.71 | 10.99 ± 2.60 | 2.44 |
| Vinblastine (nM) | 0.61 ± 0.09 | 9.27 ± 3.22 | 15.20 | 1.75 ± 0.21 | 10.20 ± 1.97 | 5.82 |
| CTR-17 (μM) | 0.38 ± 0.07 | 0.65 ± 0.16 | 1.71 | 1.14 ± 0.04 | 0.52 ± 0.10 | 0.45 |
| CTR-20 (μM) | 0.10 ± 0.02 | 0.25 ± 0.03 | 2.50 | 1.95 ± 0.01 | 0.13 ± 0.01 | 0.13 |

* KB-C-1 (cervical cancer) and SW-1271 (lung cancer) cell s are multidrug naïve, and KB-C2 (cervical cancer) and H69AR (lung cancer) are multidrug-resistant cancer cells.

TABLE 8
The combination of CTR compounds and paclitaxel show synergistic
effects on MDR1-overexpressing KB-C-2 cells.

| | | | | |
|---|---|---|---|---|
| | | | Combination index | |
| Treatment | Ratio | Cell survival rates^{b} | (CI) | Conclusions |

| | | | | |
|---|---|---|---|---|
| CTR-17 only | IC₅₀ (0.65 μM) | 53.2 ± 12.6 | NA^{a} | NA |
| | 0.5IC₅₀ (0.325 μM) | 80.0 ± 11.4 | NA | NA |
| CTR-20 only | IC₅₀ (0.25 μM) | 56.8 ± 8.4 | NA | NA |
| | 0.5IC₅₀ (0.125 μM) | 76.0 ± 9.6 | NA | NA |
| Paclitaxel only | IC₅₀ (23 nM) | 48.4 ± 5.8 | NA | NA |
| | 0.5IC₅₀ (11.5 nM) | 75.9 ± 7.7 | NA | NA |
| | 0.25IC₅₀ (5.75 nM) | 89.5 ± 6.0 | NA | NA |
| | 0.125IC₅₀ (2.875 nM) | 99.6 ± 0.3 | NA | NA |
| CTR-17 + | 0.5IC₅₀:IC₅₀ | 15.7 ± 2.8 | 0.87 | Moderately synergistic |
| Paclitaxel | 0.5IC₅₀:0.5IC₅₀ | 29.2 ± 7.5 | 0.79 | Moderately synergistic |
| | IC₅₀:IC₅₀ | 4.7 ± 1.6 | 0.71 | Substantially synergistic |
| | IC₅₀:0.5IC₅₀ | 12.1 ± 3.6 | 0.76 | Moderately synergistic |
| | IC₅₀:0.25IC₅₀ | 18.3 ± 6.2 | 0.73 | Substantially synergistic |
| | IC₅₀:0.125IC₅₀ | 26.9 ± 9.2 | 0.77 | Moderately synergistic |
| CTR-20 + | 0.5IC₅₀:IC₅₀ | 16.3 ± 2.6 | 0.72 | Substantially synergistic |
| Paclitaxel | 0.5IC₅₀:0.5IC₅₀ | 39.1 ± 6.1 | 0.89 | Moderately synergistic |
| | IC₅₀:IC₅₀ | 9.3 ± 1.9 | 0.69 | Substantially synergistic |
| | IC₅₀:0.5IC₅₀ | 16.0 ± 2.0 | 0.69 | Substantially synergistic |
| | IC₅₀:0.25IC₅₀ | 31.4 ± 2.9 | 0.86 | Moderately synergistic |
| | IC₅₀:0.125IC₅₀ | 42.7 ± 3.0 | 0.95 | Slightly synergistic |

^{a}NA, not applicable
^{b}Values are the mean value of triplicates of at least four independent experiments.

TABLE 9
The combination of CTR compounds and paclitaxel show synergistic
effects on MDR1-overexpressing MDA-MB231TaxR cells.

| Treatment | Ratio | Cell survival rate^{a} | CI^{b} | Conclusion |
|---|---|---|---|---|
| CTR-20 only | 0.3 μM | 63.7 ± 2.52 | NA^{c} | NA |
| CTR-21 only | 23.4 nM | 52.10 ± 3.71 | NA | NA |
| CTR-32 only | 23.4 nM | 53.07 ± 6.50 | NA | NA |
| Paclitaxel only | 300 nM | 57.75 ± 3.78 | NA | NA |
| | 150 nM | 82.41 ± 5.00 | NA | NA |
| | 75.0 nM | 90.98 ± 4.49 | NA | NA |
| | 37.5 nM | 90.28 ± 3.58 | NA | NA |
| | 18.75 nM | 95.84 ± 1.86 | NA | NA |
| CTR-20 + | 0.3 μM + 300 nM | 33.27 ± 1.86 | 0.96 | Additive |
| paclitaxel | 0.3 μM + 150 nM | 37.97 ± 3.02 | 0.72 | Moderately synergistic |
| | 0.3 μM + 75.0 nM | 40.66 ± 3.88 | 0.63 | Synergistic |
| | 0.3 μM + 37.5 nM | 44.37 ± 1.38 | 0.58 | Synergistic |
| CTR-21 + | 23.4 nM + 300 nM | 32.31 ± 2.49 | 0.55 | Synergistic |
| paclitaxel | 23.4 nM + 150 nM | 39.05 ± 2.04 | 0.53 | Synergistic |
| | 23.4 nM + 75.0 nM | 43.30 ± 4.93 | 0.56 | Synergistic |
| | 23.4 nM + 37.5 nM | 41.80 ± 1.92 | 0.38 | Synergistic |
| | 23.4 nM + 18.75 nM | 42.24 ± 0.93 | 0.35 | Synergistic |
| CTR-32 + | 23.4 nM + 300 nM | 35.90 ± 0.55 | 0.61 | Synergistic |
| paclitaxel | 23.4 nM + 150 nM | 36.92 ± 1.13 | 0.36 | Synergistic |
| | 23.4 nM + 75.0 nM | 44.03 ± 1.51 | 0.35 | Synergistic |
| | 23.4 nM + 37.5 nM | 46.18 ± 0.42 | 0.30 | Synergistic |
| | 23.4 nM + 18.75 nM | 47.76 ± 0.28 | 0.27 | Synergistic |

^{a}Values are the mean value of triplicates of at least three independent experiments
^{b}CI: Combinational index.
^{c}NA: not applicable

TABLE 10
The combination of CTR-20 and ABT-737 shows strong
synergistic effects against MDA-MB231 cells.

| Treatment | Ratio | Cell survival rate^{a} | CI^{b} | Conclusion |
|---|---|---|---|---|
| ABT-737 only | 6.25 μM | 91.01 ± 9.81 | NA^{c} | NA |
| | 3.125 μM | 98.61 ± 3.93 | NA | NA |
| CTR-20 only | 0.4 μM | 26.38 ± 4.73 | NA | NA |
| | 0.2 μM | 41.07 ± 4.58 | NA | NA |
| | 0.1 μM | 70.86 ± 4.17 | NA | NA |
| CTR-20 + ABT-737 | 0.4 μM + 6.25 μM | −15.16 ± 2.11 | 0.10 | Strongly synergistic |
| | 0.2 μM + 6.25 μM | −10.41 ± 2.84 | 0.07 | Very strongly synergistic |
| | 0.1 μM + 6.25 μM | 42.30 ± 3.38 | 0.57 | Synergistic |
| | 0.4 μM + 3.125 μM | −10.98 ± 3.17 | 0.09 | Very strongly synergistic |
| | 0.2 μM + 3.125 μM | 13.18 ± 4.91 | 0.26 | Strongly synergistic |
| | 0.1 μM + 3.125 μM | 61.82 ± 3.98 | 0.84 | Moderately synergistic |

^{a}Values are the mean value of triplicates of at least three independent experiments
^{b}CI: Combinational index.
^{c}NA: not applicable

TABLE 11
Typical protocol for the study of CTR compounds using xenograft mice (ATH490).

| Treatment | Dosage | Frequency | Route | Notes |
|---|---|---|---|---|
| Sham control | Highest volume | Every 3 days | Intraperitoneal (I.P.) | Vehicle only |
| Paclitaxel | 10 mg/kg B.W.^{a} | Once/week | Intravenous (I.V.) | |
| CTR-17 (30) | 30 mg/kg B.W. | Every 3 days | I.P. | |
| CTR-20 (30) | 30 mg/kg B.W. | Every 3 days | I.P. | |
| Paclitaxel (5), | Paclitaxel 5 mg/kg B.W. & | Once/week | Paclitaxel (I.V.) & | Paclitaxel was given 24 |
| CTR-17 (15) | CTR-17 15 mg/kg B.W. | | CTR-17 (I.P.) | hours prior to CTR-17 |
| Paclitaxel (5), | Paclitaxel 5 mg/kg B.W. & | Once/week | Paclitaxel (I.V.) & | Paclitaxel was given 24 |
| CTR-20 (15) | CTR-20 15 mg/kg B.W | | CTR-20 (I.P.) | hours prior to CTR-20 |

^{a}B.W. denotes body weight.

TABLE 12
Antitumor activity of CTR-17 and CTR-20, alone or in combination with paclitaxel.

| | Day 0 | Day 6 | Day 14 | Day 17 | Day 20 | Day 24 | Day 27 | Day 30 |
|---|---|---|---|---|---|---|---|---|
| Sham control | 89.61 ± | 105.03 ± | 136.66 ± | 162.62 ± | 192.69 ± | 268.08 ± | 426.87 ± | 557.66 ± |
| | 8.97 | 15.90 | 16.87 | 15.29 | 16.56 | 37.85 | 7.57 | 24.72 |
| Tax^{a} (10 mg^{b}) | 91.84 ± | 90.11 ± | 94.66 ± | 92.33 ± | 81.66 ± | 95.61 ± | 139.41 ± | 170.36 ± |
| | 6.52 | 17.42 | 31.77 | 31.56 | 30.09 | 29.69 | 24.96 | 40.07 |
| CTR-17 (30 mg) | 86.71 ± | 63.99 ± | 90.07 ± | 128.36 ± | 134.76 ± | 160.86 ± | 189.45 ± | 209.84 ± |
| | 5.42 | 3.85 | 21.30 | 20.65 | 26.46 | 37.30 | 47.61 | 56.45 |
| CTR-20 (30 mg) | 92.51 ± | 69.89 ± | 84.64 ± | 97.11 ± | 97.22 ± | 106.26 ± | 124.88 ± | 140.63 ± |
| | 10.45 | 8.22 | 5.09 | 8.59 | 13.40 | 26.05 | 34.85 | 38.00 |
| Tax (5 mg) plus | 91.38 ± | 74.70 ± | 70.05 ± | 65.42 ± | 60.67 ± | 70.80 ± | 81.88 ± | 108.37 ± |
| CTR-17 (15 mg) | 13.00 | 17.35 | 13.74 | 23.79 | 21.29 | 17.46 | 19.85 | 35.30 |
| Tax (5 mg) plus | 95.35 ± | 61.76 ± | 65.65 ± | 51.14 ± | 54.36 ± | 51.19 ± | 47.20 ± | 65.71 ± |
| CTR-20 (15 mg) | 3.46 | 5.76 | 16.32 | 12.37 | 8.13 | 6.90 | 13.15 | 22.00 |

^{a}Tax: paclitaxel.
^{b}mg per kg of body weight.

TABLE 13
Antitumor activity of CTR-18 and CTR-19.

| | D 0 | D 6 | D 14 | D 17 | D 20 | D 24 | D 27 | D 30 |
|---|---|---|---|---|---|---|---|---|
| Control | 89.61 ± | 105.03 ± | 136.66 ± | 162.62 ± | 192.69 ± | 268.08 ± | 426.87 ± | 557.66 ± |
| | 8.97 | 15.90 | 16.87 | 15.29 | 16.56 | 37.85 | 7.57 | 24.72 |
| CTR-18 (30 mg) | 92.51 ± | 111.09 ± | 138.10 ± | 145.48 ± | 166.84 ± | 143.78 ± | 141.52 ± | 253.37 ± |
| | 6.73 | 23.55 | 35.18 | 34.28 | 48.43 | 23.27 | 16.66 | 77.10 |
| CTR-19 (30 mg) | 92.51 ± | 84.79 ± | 99.68 ± | 92.27 ± | 115.39 ± | 124.76 ± | 143.62 ± | 200.77 ± |
| | 12.97 | 10.38 | 9.41 | 8.19 | 8.62 | 9.48 | 20.22 | 50.95 |
| Tax^{a} (5 mg^{b}) plus | 84.60 ± | 78.81 ± | 96.61 ± | 88.40 ± | 92.31 ± | 109.97 ± | 125.23 ± | 178.95 ± |
| CTR-18 (15 mg) | 7.36 | 4.61 | 12.22 | 4.90 | 16.20 | 20.81 | 19.03 | 29.63 |
| Tax (5 mg) plus | 93.84 ± | 96.25 ± | 102.59 ± | 97.72 ± | 101.44 ± | 143.28 ± | 189.18 ± | 206.37 ± |
| CTR-19 (15 mg) | 8.15 | 5.15 | 13.81 | 7.98 | 18.94 | 28.02 | 41.47 | 44.05 |

^{a}Tax: paclitaxel.
^{b}mg per kg of body weight.

TABLE 14
Analysis of liver toxicity by AST and ALT.

| Treatment | ALT^{a} (IU/L) | AST^{b}(IU/L) |
|---|---|---|
| Untreated | 54.08 ± 4.98 | 113.08 ± 9.13 |
| Sham Control | 64.03 ± 4.04 | 112.46 ± 4.74 |
| Paclitaxel (10 mg/kg) | 52.02 ± 2.59 | 111.14 ± 13.42 |
| CTR-17, 20 mg/kg | 63.59 ± 9.73 | 101.00 ± 10.19 |
| CTR-17, 30 mg/kg | 64.67 ± 5.26 | 114.13 ± 6.36 |
| CTR-20, 20 mg/kg | 56.77 ± 5.34 | 119.19 ± 4.03 |
| CTR-20, 30 mg/kg | 50.50 ± 9.96 | 118.22 ± 17.00 |
| Paclitaxel (5), CTR-17 (15) | 46.01 ± 0.42 | 106.70 ± 13.41 |
| Paclitaxel (5), CTR-20 (15) | 59.62 ± 7.25 | 107.67 ± 13.22 |

^{a}ALT: Alanine transaminase.
^{b}AST: aspartate aminotransferase.

TABLE 15
Toxicology analysis of spleen.

| Treatment | White pulp (WP) | Red Pulp (RP) | Capsule |
|---|---|---|---|
| Untreated | Normal | Normal | Normal |
| Vehicle Control | Normal | Normal | Normal |
| Paclitaxel (10 mg/kg) | Less but bigger | Myeloid | Normal |
| | | elements: ↑↑^{a} | |
| CTR-17, 30 mg/kg | Less but bigger | Myeloid | Normal |
| | | elements: ↑ | |
| CTR-20, 30 mg/kg | Less but bigger | Myeloid | Normal |
| | | elements: ↑ | |
| Tax (5), CTR-17 (15) | Normal | Normal | Normal |
| Tax (5), CTR-20 (15) | Normal | Normal | Normal |

^{a}Double and single upward arrows indicate highly and moderately increased, respectively

What is claimed is:

1. A compound of Formula I: 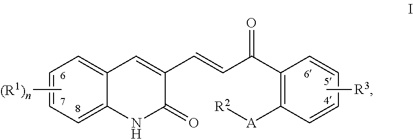
wherein
A is O or S;
n is 0, 1, 2 or 3;
when n is 1, R¹ is halo, C₁₋₆alkyl, C₂₋₆alkenyl or —X—C₁₋₆alkyl;
when n is 2 or 3, each R¹ is independently halo, C₁₋₆alkyl, C₂₋₆alkenyl or —X—C₁₋₆alkyl; or
two R¹ together form a methylenedioxy group that is attached to two adjacent ring carbon atoms;
R² is C₁₋₆alkyl or C₁₋₆haloalkyl;
R³ is absent or is halo, —X—C₁₋₆alkyl or —X—C₁₋₆haloalkyl; and
each X is independently O or S,
or a pharmaceutically acceptable salt, solvate and/or prodrug thereof.

2. The compound of claim 1, wherein A is O.

3. The compound of claim 1, wherein R³ is absent.

4. The compound of claim 1, wherein R² is methyl.

5. The compound of claim 1, wherein n is 0.

6. The compound of claim 1, wherein n is 1 and R¹ is 6-OCH₃, 7-OCH₃, 8-OCH₃, 6-OC₂H₅, 6-SCH₃, 7-SCH₃, 6-CH₃, 6-C₂H₅, 6-F, 6-Cl, 6-Br, 7-F, 7-Cl or 7-Br, optionally wherein R¹ is 6-CH₃, 6-OCH₃ or 7-OCH₃.

7. The compound of claim 1, wherein n is 2 and R¹ is 6,7-diCH₃, 6,7-diOCH₃ or 6,7-O—CH₂—O—.

8. The compound of claim 1, wherein n is 3 and R¹ is 5,6,7-triOCH₃.

9. The compound of claim 1, wherein the compound is selected from: 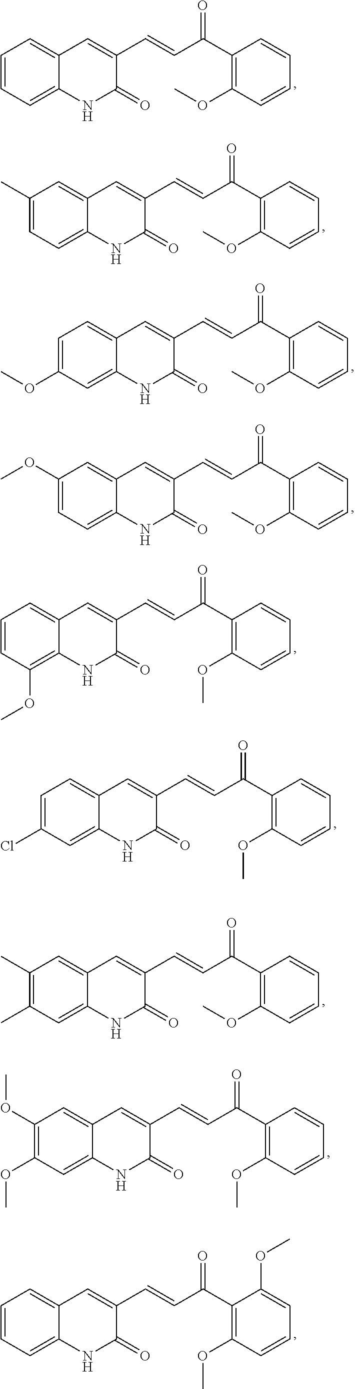 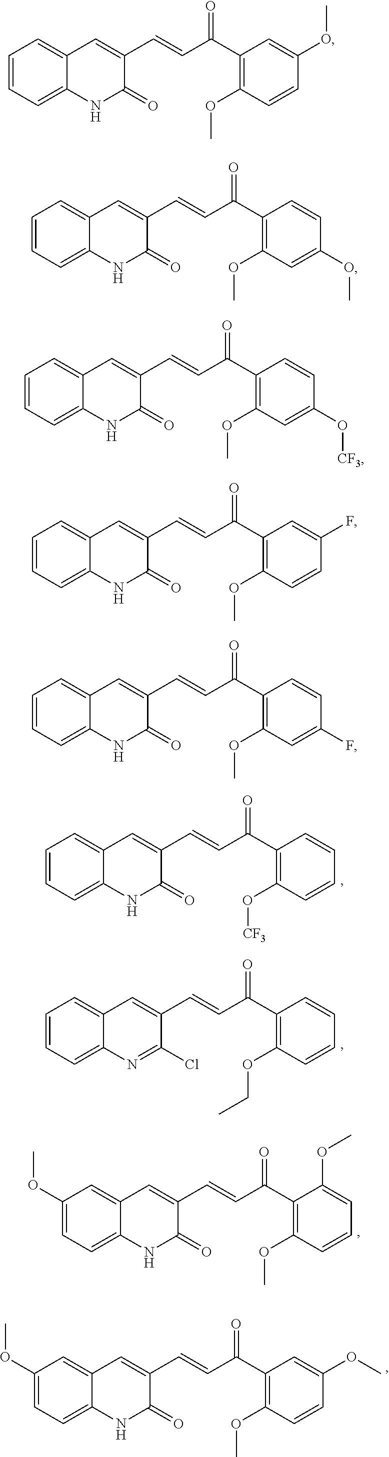 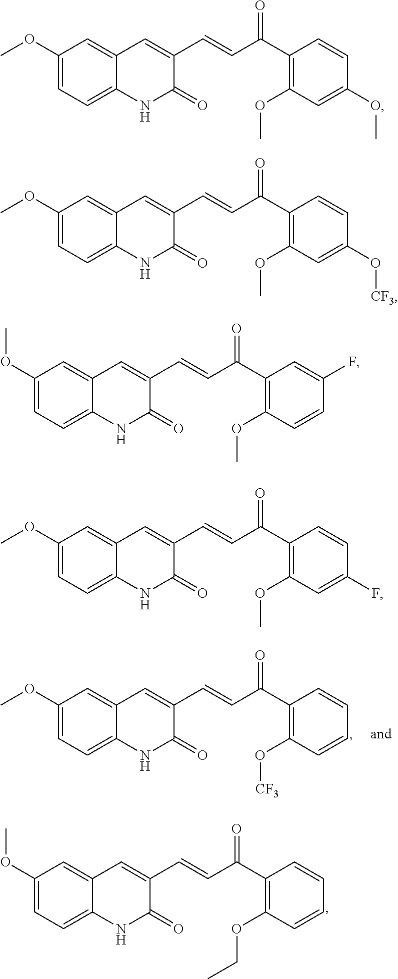
or a pharmaceutically acceptable salt, solvate and/or prodrug thereof.

10. The compound of claim 9, wherein the compound is:

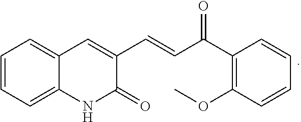 [SMILES: COc1ccccc1C(=O)/C=C/c1cc2ccccc2[nH]c1=O]

11. The compound of claim 9, wherein the compound is:

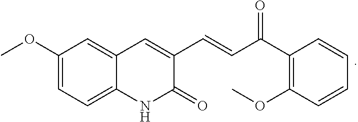 [SMILES: COc1ccc2[nH]c(=O)c(/C=C/C(=O)c3ccccc3OC)cc2c1]

12. A pharmaceutical composition comprising one or more compounds of claim 1 and a pharmaceutically acceptable carrier.

13. A method of treating cancer comprising administering one or more compounds of claim 1 to a subject in need thereof, wherein the cancer is breast cancer, leukemia, cervical cancer, brain cancer, lung cancer, bladder cancer, kidney cancer, multiple myeloma or other blood cancers, colorectal cancer, CNS cancer, melanoma, ovarian cancer or prostate cancer.

14. (canceled)

15. The method of claim 13, wherein the cancer comprises colchicine-resistant, paclitaxel-resistant, bortezomib-resistant, vinblastine-resistant and/or multidrug-resistant tumor cells.

16. The method of claim 13, wherein the one or more compounds of claim 1 are administered in combination with one or more other anticancer agents.

17. The method of claim 16, wherein the other anticancer agents are selected from the group consisting of mitotic inhibitors, optionally paclitaxel; bcl2 inhibitors, optionally ABT-737; proteasome inhibitors, optionally bortezomib or calfilzomib; signal transduction inhibitors, optionally gefitinib, erlotinib, dasatinib, imatinib or sunitinib; inhibitors of DNA repair, optionally iniparib, temozolomide or doxorubicin; and alkylating agents, optionally cyclophosphamide.

18. The method of claim 17, wherein the other anticancer agent is paclitaxel.

19. The method of claim 16, wherein the dosage of the one or more compounds of claim 1 is less than the dosage of the one or more compounds of claim 1 when administered alone.

20. The method of claim 19, wherein the dosage of the one or more compounds of claim 1 is one half the dosage of the one or more compounds of claim 1 when administered alone.

21. The method of claim 16, wherein the dosage of the other anticancer agent is less than the dosage of the other anticancer agent when administered alone.

22. The method of claim 21, wherein the dosage of the other anticancer agent is one half the dosage of the other anticancer agent when administered alone.
